(19) United States
(12) Patent Application Publication
(10) Pub. No.: US 2010/0173960 A1
(43) Pub. Date: Jul. 8, 2010

(54) The Combination of a Cyclohexanehexol and a NSAID for the Treatment of Neurodegenerative Diseases

(76) Inventors: Antonio Cruz, Toronto (CA); Carl Damiani, Ontario (CA)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/438,572

(22) PCT Filed: Sep. 20, 2007
(86) PCT No.: PCT/CA07/01678

§ 371 Date: Dec. 7, 2009

Related U.S. Application Data
(60) Provisional application No. 60/846,366, filed on Sep. 21, 2006.

(51) Int. Cl. A61K 31/423; A61K 31/192; A61K 31/407; A61K 31/381; A61K 31/403; A61K 31/402; A61K 31/4035; A61P 25/28; A61P 25/16; A61P 25/08

(52) U.S. Cl. 514/375; 514/570; 514/413; 514/448; 514/411; 514/429; 514/416

(57) ABSTRACT

The invention relates generally to novel compositions and methods comprising one or more cyclohexanehexol and one or more NSAID. The compositions and methods provide beneficial effects in the treatment and prevention of diseases involving a disorder in protein folding and/or aggregation, and/or amyloid formation, deposition, accumulation, or persistence, in particular neurodegenerative diseases.

### FIELD OF THE INVENTION

The invention provides compositions, conjugates, and methods comprising a cyclohexanehexol and a non-steroidal anti-inflammatory drug (NSAID), and uses thereof.

### BACKGROUND OF THE INVENTION

A number of therapeutics have been developed to treat neurodegenerative diseases such as Alzheimer's disease, including acetylcholine esterase inhibitors (e.g., tacrine, donepezil, rivastigmine, and galantamine), NMDA receptor antagonists such as memantine, and non-steroidal anti-inflammatory drugs (NSAIDs). Some of the therapeutics alleviate symptoms of Alzheimer's disease but none prevent or alter the disease. In addition, the therapeutics may have undesirable side effects. In view of the present interest in the treatment or prevention of neurodegenerative diseases improved compositions and methods for treating neurodegenerative diseases are desired and would be a welcome contribution to the art.

### SUMMARY OF THE INVENTION

The invention generally relates to compositions, conjugates, and methods (e.g., combination therapies) comprising one or more cyclohexanehexol and one or more NSAID, in particular R-NSAID. In an aspect these compositions, conjugates, and methods provide beneficial effects in the prevention, interevention or treatment of diseases for which a cyclohexanehexol and/or NSAID, in particular R-NSAID, have a therapeutic effect, including diseases characterized by amyloid deposition, in particular Alzheimer's disease and similar diseases.

A composition, conjugate, or method (e.g. combination therapy) comprising a cyclohexanehexol and a NSAID, in particular R-NSAID, may improve tolerance to the therapy with a reduced risk of side effects that may result from higher doses or longer term monotherapies (i.e., therapies with each compound alone). A treatment of the invention may permit the use of lower doses of each compound (in particular aspects, lower doses of a NSAID, in particular R-NSAID), preferably with reduced adverse effects of a compound. A suboptimal dosage may provide an increased margin of safety, and may also reduce the cost of a drug necessary to achieve prophylaxis and therapy. In addition, a treatment utilizing a single combination dosage unit may provide increased convenience and may result in enhanced compliance. Other advantages of a composition, conjugate, or combination therapy may include higher stability towards degradation and metabolism, longer duration of action, and/or longer duration of action or effectiveness at particularly low doses.

In an aspect, the invention contemplates a composition, in particular a pharmaceutical composition, comprising a cyclohexanehexol and a NSAID, in particular R-NSAID. A pharmaceutical composition may optionally comprise a pharmaceutically acceptable carrier, excipient, or vehicle.

More specifically, the invention provides a composition for treating and delaying the onset of neurodegenerative diseases. A composition of the invention has one or more cyclohexanehexol and one or more NSAID, in particular R-NSAID, and optionally one or more pharmaceutically acceptable carriers.

The invention also contemplates a composition, comprising one or more cyclohexanehexol and one or more NSAID, in particular R-NSAID, which provide beneficial effects relative to each compound alone. The beneficial effects provided by a composition of the invention can include enhanced therapeutic effects, in particular sustained therapeutic effects. More particularly, a composition, conjugate, or method (e.g. combination therapy) comprising a cyclohexanehexol and a NSAID, in particular R-NSAID, may significantly reduce beta amyloid deposits in the brain (beta-amyloid plaques) and/or cerebral blood vessels (beta amyloid angiopathy). The beneficial effects provided by a composition of the invention may include inhibition, reduction, or reversal of Aβ fibril assembly or aggregation, Aβ toxicity, abnormal protein folding, aggregation, amyloid formation, deposition, accumulation or persistence, and/or amyloid lipid interactions, and/or acceleration of disassembly of preformed fibrils. Beneficial effects may also include the reduction of at least one symptom of a disease, or preventing an increase (or slowing the rate of increase) in or delaying the onset of, at least one symptom of a disease.

A composition of the invention can be in a form that results in disruption of aggregating Aβ, reduced cerebral accumulation of amyloid β, deposition of cerebral amyloid plaques, soluble Aβ oligomers in the brain and/or body fluids (especially Aβ₄₂), glial activity, inflammation, and/or cognitive decline in the subject. A composition can have increased bioavailability (absorbed more rapidly and to a higher degree) or provide enhanced therapeutic effects.

A treatment of the invention may be sustained over several days, weeks, months or years thereby having a major beneficial impact on the severity of the disease and its complications. Therefore, the invention also provides a composition for the treatment of a disease comprising a therapeutically effective amount of a cyclohexanehexol and a NSAID, in particular R-NSAID, to provide a sustained beneficial effect following treatment in a pharmaceutically acceptable carrier, excipient, or vehicle.

In an aspect, a pharmaceutical composition is provided comprising a scyllo-inositol compound and R-NSAID selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-etodolac and R-benoxaprofen.

In a specific aspect of the invention, the cylcohexanehexol and NSAID, in particular R-NSAID, are adapted for administration to a subject to provide beneficial effects to treat a disease. In an embodiment, the composition is in a form such that administration to a subject results in reduction in Aβ levels (e.g., Aβ levels) in the subject for a sustained period of time after cessation of treatment.

In another aspect, the invention features a composition comprising one or more cyclohexanehexol and one or more NSAID, in dosages effective for inhibiting, reducing, reversing, or disrupting Aβ fibril assembly or aggregation, Aβ toxicity, abnormal protein folding, aggregation, amyloid formation, deposition, accumulation or persistence, and/or amyloid lipid interactions, and/or acceleration of disassembly of preformed fibrils, in particular, for a sustained period following administration of the cyclohexanehexol. In embodiments of the invention the NSAID is a R-NSAID selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-etodolac and R-benoxaprofen.

In another aspect, the invention features a composition comprising a cyclohexanehexol and a NSAID, in particular R-NSAID, in a dosage effective for disrupting aggregation of Aβ, increasing inhibition of long term potentiation induced by Aβ oligomers and/or maintenance of synaptic function, and/or for reducing cerebral accumulation of amyloid β, deposition of cerebral amyloid plaques, soluble Aβ oligomers (e.g., Aβ₄₂ oligomers) in the brain and/or body fluids, glial activity, inflammation, and/or cognitive decline in the subject, in particular for a sustained period following administration of the composition.

The invention provides a composition, in particular a pharmaceutical composition, comprising a cyclohexanehexol and NSAID, in particular R-NSAID, which provides beneficial effects in the treatment of a disease disclosed herein, in particular a disorder in protein folding and/or aggregation, and/or amyloid formation, deposition, accumulation, or persistence.

In an aspect the invention provides a combination of a cyclohexanehexol and a

NSAID, in particular R-NSAID, that provides beneficial effects in the treatment of conditions for which either a cyclohexanehexol or NSAID, in particular a R-NSAID, have been demonstrated to have a therapeutic effect, including but not limited to neurodegenerative diseases such as Alzheimer's disease, and similar diseases.

A cyclohexanehexol and a NSAID, in particular R-NSAID, in a composition or combination of the invention may be in a ratio selected to augment the activity of the cyclohexanehexol and/or NSAID, in particular to provide a beneficial effect.

Combinations of a cyclohexanehexol and a NSAID, in particular R-NSAID, in compositions of the invention may be selected to provide unexpectedly additive effects or greater than additive effects i.e., synergistic effects.

In an aspect, the invention provides a pharmaceutical composition comprising a cyclohexanehexol and a NSAID, in particular R-NSAID, in combination with a pharmaceutically acceptable carrier, excipient or vehicle, wherein the amounts of cyclohexanehexol and NSAID, in particular R-NSAID, are selected to provide an additive or synergistic beneficial effect in preventing or treating a disease disclosed herein.

In an aspect, the invention provides a pharmaceutical composition comprising a cyclohexanehexol and a NSAID, in particular R-NSAID, in combination with a pharmaceutically acceptable carrier, excipient or vehicle, wherein the amounts of cyclohexanehexol and NSAID, in particular R-NSAID, are selected to provide a beneficial effect in preventing or reducing aggregation of Aβ, maintaining synaptic function, and/or reducing Aβ load, as a combined preparation for simultaneous, separate or sequential use in treatment of neurodegenerative diseases including Alzheimer's disease and similar diseases.

In an aspect, the invention provides a pharmaceutical composition comprising a cyclohexanehexol and a NSAID, in particular R-NSAID, in combination with a pharmaceutically acceptable carrier, excipient or vehicle, wherein the amounts of cyclohexanehexol and NSAID, in particular R-NSAID, are selected to provide an additive effect in preventing or reducing aggregation of Aβ, maintaining synaptic function, and/or reducing Aβ load, as a combined preparation for simultaneous, separate or sequential use in treatment of neurodegenerative diseases including Alzheimer's disease and similar diseases.

In an aspect, the invention provides a pharmaceutical composition comprising a cyclohexanehexol and a NSAID, in particular R-NSAID, in combination with a pharmaceutically acceptable carrier, excipient or vehicle, wherein the amounts of cyclohexanehexol and NSAID, in particular R-NSAID, are selected to provide a synergistic effect in preventing or reducing aggregation of Aβ, maintaining synaptic function, and/or reducing Aβ load, as a combined preparation for simultaneous, separate or sequential use in treatment of neurodegenerative diseases including Alzheimer's disease and similar diseases.

In an aspect, the invention provides a pharmaceutical composition comprising an amount of a cyclohexanehexol and an amount of a NSAID, in particular R-NSAID, wherein said composition achieves a synergistic effect for treating a neurodegenerative disease in a mammal in need thereof.

In an aspect, the invention provides a pharmaceutical composition comprising a cyclohexanehexol and a NSAID, in particular R-NSAID, in combination with a pharmaceutically acceptable carrier, excipient or vehicle, wherein the cyclohexanehexol and NSAID, in particular R-NSAID, are present in therapeutically effective amounts at or adjacent to a site of administration of the pharmaceutical composition and at a time of administration sufficient to provide a beneficial effect, in particular a synergistic therapeutic effect on preventing or reducing aggregation of Aβ, maintaining synaptic function, and/or reducing Aβ load.

In an aspect, the invention provides a pharmaceutical composition comprising a cyclohexanehexol and a NSAID, in particular R-NSAID, in respective proportions so as to provide a beneficial effect, in particular a synergistic therapeutic effect, on preventing or reducing aggregation of Aβ, maintaining synaptic function, and/or reducing Aβ load, as a combined preparation for simultaneous, separate or sequential use in treatment of neurodegenerative diseases, including Alzheimer's disease and similar diseases.

The compositions of the invention can be provided with two active ingredients combined together in a single dose with one or more pharmaceutically acceptable carriers, or as two separate pharmaceutical compositions contained in appropriate packaging indicating the two pharmaceuticals are to be used together.

In an aspect, the invention provides a pharmaceutical composition in separate containers and intended for simultaneous, separate or sequential administration to a subject especially to provide beneficial effects, comprising a cyclohexanehexol and a NSAID, in particular R-NSAID, both optionally together with pharmaceutically acceptable carriers, excipients, or vehicles.

The invention provides a conjugate comprising a cyclohexanehexol interacting with or linked to a NSAID, in particular R-NSAID. A conjugate can provide the beneficial effects described herein.

The invention also provides methods for preparing compositions and conjugates of the invention, in particular compositions and conjugates with beneficial effects.

In an aspect, the invention provides a method of preparing a pharmaceutical composition, in particular a stable pharmaceutical composition, comprising one or more cyclohexanehexol and one or more NSAID, in particular R-NSAID. In a particular aspect, the method can comprise mixing one or more cyclohexanehexol and one or more NSAID, in particular R-NSAID, and a pharmaceutically acceptable carrier, excipient, or vehicle, in particular, a pharmaceutically acceptable carrier, excipient, or vehicle effective to physically stabilize the cyclohexanehexol(s) and/or one or more NSAID, in particular R-NSAID. After compositions have been prepared, they can be placed in an appropriate container(s) and labeled for treatment of an indicated condition. For administration of a composition of the invention, such labeling would include amount, frequency, and method of administration.

The invention also contemplates the use of one or more cyclohexanehexol and one or more NSAID, in particular R-NSAID, composition, conjugate, or combination treatment of the invention for preventing, and/or ameliorating disease severity, disease symptoms, and/or periodicity of recurrence of a disease disclosed herein.

The invention also relates to the prevention and treatment, in a subject, of diseases, (e.g. neurodegenerative diseases), using a cyclohexanehexol and a NSAID, in particular R-NSAID, composition, combination treatment, and/or conjugate of the invention.

In particular, the invention provides a method for treating and/or preventing a disease in a subject comprising administering to the subject a therapeutically effective amount of one or more cyclohexanehexol and one or more NSAID, in particular R-NSAID. In an aspect the invention provides a treatment which results in sustained beneficial effects following treatment.

In an aspect, the invention provides a method of treating a disease disclosed herein, in particular a neurodegenerative disease in a subject in need thereof comprising the step of administering to the subject a therapeutically effective amount of a synergistic composition of a cyclohexanehexol and a NSAID, in particular R-NSAID.

In an aspect, the invention provides a method for the prevention and/or intervention of a disease disclosed herein in a subject comprising administration of at least one cyclohexanehexol and at least one NSAID, in particular R-NSAID, or a composition or conjugate of the invention to the subject.

The invention provides a method of treating a disease comprising administering at least one cyclohexanehexol and at least one NSAID, in particular R-NSAID, a composition, combination treatment or conjugate of the invention to a subject in need thereof to thereby produce beneficial effects. In an embodiment, the compounds, composition, and/or conjugate are administered orally or systemically. In an embodiment, the invention provides a method for the prevention and/or intervention of a disease discussed herein in a subject comprising administration of at least one cyclohexanehexol and at least one NSAID, in particular R-NSAID, or a composition of the invention to a subject in need thereof to provide beneficial effects.

In another embodiment, the invention provides a method for the prevention and/or intervention of a disease disclosed herein in a subject comprising co-administering at least one cyclohexanehexol and at least one NSAID, in particular R-NSAID, or a composition of the invention to a subject in need thereof.

In a further aspect, the invention provides a method for amelioriating progression of a disease or obtaining a less severe stage of a disease in a subject suffering from such disease (e.g. Alzheimer's disease) comprising administering a therapeutically effective amount of at least one cyclohexanehexol and at least one NSAID, in particular R-NSAID, or a composition of the invention.

The invention relates to a method of delaying the progression of a disease (e.g.

Alzheimer's disease) comprising administering a therapeutically effective amount of at least one cyclohexanehexol and at least one NSAID, in particular R-NSAID, or a composition of the invention.

The invention also relates to a method of increasing survival of a subject suffering from a disease comprising administering a therapeutically effective amount of at least one cyclohexanehexol and at least one NSAID, in particular R-NSAID, or a composition of the invention.

In an embodiment, the invention relates to a method of improving the lifespan of a subject suffering from a disease disclosed herein (e.g., Alzheimer's disease) comprising administering a therapeutically effective amount of at least one cyclohexanehexol and at least one NSAID, in particular R-NSAID, or a composition of the invention.

In another embodiment, the invention provides a therapeutic method which comprises identifying a patient diagnosed with a neurodegenerative disorder (such as Alzheimer's disease or MCI) and treating the patient with an effective amount of one or more cyclohexanehexol and one or more NSAID, in particular R-NSAID.

An aspect of the invention provides a method for treating a neurodegenerative disorder. According to the method of this aspect, a therapeutically effective amount of one or more cyclohexanehexol and one or more NSAID, in particular R-NSAID, is administered to an individual in need of such treatment. An individual in need of such treatment may have a neurodegenerative disorder, a predisposition to a neurodegenerative disorder, and or desire prophylaxis against or a delay in such disorders.

In specific aspects of the invention, a neurodegenerative disorder is selected from the group consisting of Alzheimer's disease, dementia, mild cognitive impairment, and tauopathies (e.g. corticobasal degeneration, frontotemporal dementia with Parkinsonism linked to chromosome 17, and progressive supranuclear palsy).

The invention has particular applications in preventing and/or treating Alzheimer's disease or MCI and other similar diseases. In an aspect, the invention provides a method for preventing and/or treating Alzheimer's disease or MCI comprising administering a therapeutically effective amount of a cyclohexanehexol and at least one NSAID, in particular R-NSAID, or a composition or conjugate of the invention. In an embodiment, the invention relates to a method of treating Alzheimer's disease comprising administering a therapeutically effective amount of one or more cyclohexanehexol and one or more NSAID, in particular R-NSAID, which upon administration to a subject with symptoms of Alzheimer's disease produces beneficial effects, in particular sustained beneficial effects. In another aspect, the invention provides a method of treating Alzheimer's disease comprising administering one or more cyclohexanehexol and one or more NSAID, in particular R-NSAID, in effective amounts for reducing at least one symptom of Alzheimer's disease or for reducing, preventing an increase (or slowing the rate of increase) in or delaying the onset of, at least one symptom of Alzheimer's disease.

An embodiment of the invention provides a method for preventing and/or treating Alzheimer's disease, the method comprising administering to a mammal in need thereof a combination of a cyclohexanehexol and a NSAID, in particular R-NSAID, in an amount sufficient to inhibit, reduce, or reverse Aβ fibril assembly or aggregation, Aβ toxicity, abnormal protein folding, aggregation, amyloid formation, deposition, accumulation or persistence, and/or amyloid lipid interactions, and/or acceleration of disassembly of preformed fibrils, thereby preventing and/or treating the disease.

The invention provides methods for treating Alzheimer's disease in a patient in need thereof by administering a composition comprising a cyclohexanehexol and a NSAID, in particular R-NSAID, composition, or conjugate of the invention in an amount sufficient to inhibit, reduce, or reverse Aβ fibril assembly or aggregation, Aβ toxicity, abnormal protein folding, aggregation, amyloid formation, deposition, accumulation or persistence, and/or amyloid lipid interactions, and/or acceleration of disassembly of preformed fibrils in the subject.

In an embodiment, the invention provides a method of reversing amyloid deposition and neuropathology after the onset of cognitive deficits and amyloid plaque neuropathology in a subject comprising administering to the subject a therapeutically effective amount of a composition of the invention.

The invention also contemplates the use of a composition comprising at least one cyclohexanehexol and at least one NSAID, in particular R-NSAID, for the preparation of a medicament for preventing and/or treating a disease. In an embodiment, the invention relates to the use of synergistically effective amounts of at least one cyclohexanehexol and at least one NSAID, in particular R-NSAID, for the preparation of a medicament for preventing and/or treating a disease. The invention additionally provides uses of a pharmaceutical composition and a conjugate of the invention in the preparation of medicaments for the prevention and/or treatment of diseases disclosed herein. In aspects of the invention, the medicaments provide beneficial effects, preferably sustained beneficial effects following treatment.

Since the present invention in part relates to a method of treatment comprising a combination of active agents which may be administered separately or as conjugates, the invention also provides a kit comprising a cyclohexanehexol and a NSAID, in particular R-NSAID, a pharmaceutical composition, or conjugate of the invention in kit form. In an aspect, the invention provides a kit comprising one or more cyclohexanehexol and one or more NSAID, in particular R-NSAID, or a pharmaceutical composition of the invention. In particular, the invention provides a kit for preventing and/or treating a disease, containing a composition comprising one or more cyclohexanehexol, one or more NSAID, in particular R-NSAID, a container(s), and instructions for use. The composition of the kit can further comprise a pharmaceutically acceptable carrier, excipient, or vehicle.

In an embodiment, the invention provides a kit for preventing and/or treating a neurodegenerative disease (e.g., Alzheimer's disease and similar diseases), containing a composition comprising a cyclohexanehexol and a NSAID, in particular R-NSAID, a container, and instructions for use. The composition of the kit can further comprise a pharmaceutically acceptable carrier.

These and other aspects, features, and advantages of the present invention should be apparent to those skilled in the art from the following detailed description.

### DETAILED DESCRIPTION OF EMBODIMENTS

Unless defined otherwise, all technical and scientific terms used herein have the same meaning as commonly understood by one of ordinary skill in the art to which this invention belongs.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to a composition containing “a compound” includes a mixture of two or more compounds.

The term “treating” refers to reversing, alleviating, or inhibiting the progress of a disease, or one or more symptoms of such disease, to which such term applies. Depending on the condition of the subject, the term also refers to preventing a disease, and includes preventing the onset, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a cyclohexanehexol and a NSAID, in particular R-NSAID, or composition of the present invention to a subject that is not at the time of administration afflicted with the disease. “Preventing” also refers to preventing the recurrence of a disease, or of one or more symptoms associated with such disease. The terms “treatment” and “therapeutically,” refer to the act of treating, as “treating” is defined above. The purpose of prevention and intervention is to combat the disease and includes the administration of the active compounds to prevent or delay the onset of the symptoms or complications, or alleviating the symptoms or complications, or eliminating the disease.

The term “administering” or “administration” refers to the process by which cyclohexanehexols and/or NSAIDs, in particular R-NSAIDs, compositions, and/or conjugates disclosed herein are delivered to a subject for treatment or prophylactic purposes. Cyclohexanehexols and/or NSAIDs, in particular R-NSAIDs, compositions, and/or conjugates are administered in accordance with good medical practices taking into account the subject's clinical condition, the site and method of administration, dosage, subject age, sex, body weight, and other factors known to the physician.

A “combination treatment” and “administering in combination” mean that the active ingredients are administered concurrently to a patient being treated. When administered in combination each component may be administered at the same time, or sequentially in any order at different points in time. Therefore, each component may be administered separately, but sufficiently close in time to provide a desired effect, in particular a beneficial, additive, or synergistic effect. The first compound may be administered in a regimen that additionally comprises treatment with the second compound. In aspects of the invention, the terms refer to the administration of a cyclohexanehexol and a NSAID, in particular R-NSAID, including separate administration of medicaments each containing one of the compounds as well as simultaneous administration whether or not the compounds are combined in one formulation or whether they are in separate formulations.

An “additive effect” of a cyclohexanehexol and a NSAID, in particular R-NSAID, refers to an effect that is equal to the sum of the effects of the two individual compounds.

A “synergistic effect” of a cyclohexanehexol and a NSAID, in particular R-NSAID, refers to an effect that is greater than the additive effect that results from the sum of the effects of the two individual compounds.

The terms “associated”, “linked”, “interact”, “interaction”, or “interacting” refer to any physical association between molecules. The terms preferably refer to a stable association between two molecules due to, for example, electrostatic, hydrophobic, ionic, hydrogen-bond interactions, or covalent interactions.

A “beneficial effect” refers to an effect of a cyclohexanehexol and/or a NSAID, in particular R-NSAID, or composition thereof in certain aspects of the invention, including favorable or enhanced pharmacological and/or therapeutic effects, and/or improved biological activity. In aspects of the invention, the beneficial effects include without limitation prevention, reduction, reversal or inhibition of Aβ fibril assembly or aggregation, Aβ toxicity, Aβ42 levels, abnormal protein folding, aggregation, amyloid formation, deposition, accumulation or persistence, and/or amyloid lipid interactions, and/or acceleration of disassembly of preformed fibrils. In particular embodiments of the invention, the beneficial effects include but are not limited to one or more of the following: disruption of aggregated Aβ or Aβ oligomers; increased or restored long term potentiation; maintenance of synaptic function; inhibition, reduction or reversal of Aβ-induced progressive cognitive decline and cerebral amyloid plaque pathology; improved cognition; increased lifespan; reduced cerebral accumulation of Aβ; reduced deposition of cerebral amyloid plaques; reduced soluble Aβ oligomers (e.g. Aβ42) in the brain and/or body fluids; reduced glial activity; reduced inflammation; and/or reduced cognitive decline. In an aspect, a beneficial effect may be a favourable characteristic of a composition/formulation of the invention and includes enhanced stability, a longer half life, and/or enhanced uptake and transport across the blood brain barrier.

The beneficial effect may be a statistically significant effect in terms of statistical analysis of an effect of a cyclohexanehexol and a NSAID, in particular R-NSAID, versus the effects without the compounds. “Statistically significant” or “significantly different” effects or levels may represent levels that are higher or lower than a standard. In embodiments of the invention, the difference may be 1.5, 2, 3, 4, 5, 6, 7, 8, 9, 10, 15, 20, 25 or 50 times higher or lower compared with the effect obtained without a cyclohexanehexol and a NSAID, in particular R-NSAID.

In an embodiment, the beneficial effect is a “sustained beneficial effect” where the beneficial effect is sustained for a prolonged period of time after a break or termination of treatment. A treatment can be sustained over several days, weeks, months or years thereby having a major beneficial impact on the severity of the disease and its complications

In aspects of the invention, a beneficial effect may be sustained for a prolonged period of at least about 2 to 4 weeks, 2 to 5 weeks, 3 to 5 weeks, 2 to 6 weeks, 2 to 8 weeks, 2 to 10 weeks, 2 to 12 weeks, 2 to 14 weeks, 2 to 16 weeks, 2 to 20 weeks, 2 to 24 weeks, 2 weeks to 12 months, 2 weeks to 18 months, 2 weeks to 24 months, or several years following treatment. The period of time a beneficial effect is sustained may correlate with the duration and timing of the treatment. A subject may be treated continuously for about or at least about 2 to 4 weeks, 2 to 6 weeks, 2 to 8 weeks, 2 to 10 weeks, 2 to 12 weeks, 2 to 14 weeks, 2 to 16 weeks, 2 weeks to 6 months, 2 weeks to 12 months, 2 weeks to 18 months, or several years, periodically or continuously.

The terms “subject”, “individual”, and “patient” refer to an animal including a warm-blooded animal such as a mammal, which is afflicted with or suspected of having or being pre-disposed to a disease disclosed herein. Mammal includes without limitation any members of the Mammalia. In general, the terms refer to a human. The terms also include domestic animals bred for food or as pets, including horses, cows, sheep, poultry, fish, pigs, cats, dogs, and zoo animals, goats, apes (e.g. gorilla or chimpanzee), and rodents such as rats and mice. Typical subjects for treatment include persons susceptible to, suffering from or that have suffered a disease disclosed herein. In particular aspects of the invention, a subject shows signs of cognitive deficits and/or amyloid plaque neuropathology. In embodiments of the invention the subjects are suspectible to, or suffer from a neurodegenerative disease such as Alzheimer's disease. In embodiments of the invention, the subject is in the late presymptomatic phase of Alzheimer's disease prior to the onset of overt cognitive deficits and amyloid neuropathology.

The term “pharmaceutically acceptable carrier, excipient, or vehicle” refers to a medium which does not interfere with the effectiveness or activity of an active ingredient and which is not toxic to the hosts to which it is administered. Acceptable carriers, excipients or vehicles may be selected from any of those commercially used in the art. By way of example, a carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers, excipients, and vehicles include but are not limited to saline, buffered saline, dextrose, water, glycerol, ethanol, and combinations thereof. The use of such media and agents for an active substance is well known in the art.

“Therapeutically effective amount” relates to the amount or dose of active compounds or a composition of the invention that will lead to one or more desired effects, in particular beneficial effects, including enhanced therapeutic effects, more particularly, one or more sustained beneficial effects. A therapeutically effective amount of a substance can vary according to factors such as the disease state, age, sex, and weight of the individual, and the ability of the substance to elicit a desired response in the individual. A dosage regimen may be adjusted to provide the optimum therapeutic response (e.g. sustained beneficial effects). For example, several divided doses may be administered daily or the dose may be proportionally reduced as indicated by the exigencies of the therapeutic situation.

“Suboptimal dose” or “suboptimal dosage” refers to a dose or dosage of an active compound which is less than the optimal dose or dosage for that compound when used in monotherapy.

A “cyclohexanehexol” is understood to refer to any compound, which fully or partially, directly or indirectly, provides one or more beneficial effects described herein and includes a compound of the formula I, II, III, IV, V or VI described herein, or an analog or derivative (e.g. functional derivative, chemical derivative or variant), salt, prodrug, polymorph, crystalline form, solvate or hydrate thereof In aspects of the invention, the cyclohexanehexol is an inositol.

A cyclohexanehexol includes a pharmaceutically acceptable salt. “Pharmaceutically acceptable salt(s),” means a salt that is pharmaceutically acceptable and has the desired pharmacokinetic properties. By pharmaceutically acceptable salts is meant those salts which are suitable for use in contact with the tissues of a subject or patient without undue toxicity, irritation, allergic response and the like, and are commensurate with a reasonable benefit/risk ratio. Pharmaceutically acceptable salts are described for example, in S. M. Berge, et al., J. Pharmaceutical Sciences, 1977, 66:1. Suitable salts include salts that may be formed where acidic protons in the compounds are capable of reacting with inorganic or organic bases. Suitable inorganic salts include those formed with alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N-methylglucamine, and the like. Suitable salts also include acid addition salts formed with inorganic acids (e.g. hydrochloride and hydrobromic acids) and organic acids (e.g. acetic acid, citric acid, maleic acid, and the alkane- and arene-sulfonic acids such as methanesulfonic acid and benezenesulfonic acid). When there are two acidic groups present, a pharmaceutically acceptable salt may be a mono-acid-mono-salt or a di-salt; and similarly where there are more than two acidic groups present, some or all of such groups can be salified.

A cyclohexanehexol includes a functional derivative, a chemical derivative, or variant. A “functional derivative” refers to a compound that possesses an activity (either functional or structural) that is substantially similar to the activity of a cyclohexanehexol disclosed herein. The term “chemical derivative” describes a molecule that contains additional chemical moieties which are not normally a part of the base molecule. The term “variant” is meant to refer to a molecule substantially similar in structure and function to a cyclohexanehexol or a part thereof. A molecule is “substantially similar” to a cyclohexanehexol if both molecules have substantially similar structures or if both molecules possess similar biological activity. The term “analog” includes a molecule substantially similar in function to a cyclohexanehexol. An “analog” can include a chemical compound that is structurally similar to another but differs slightly in composition. Differences include without limitation the replacement of an atom or functional group with an atom or functional group of a different element. Analogs and derivatives may be identified using computational methods with commercially available computer modeling programs.

A cyclohexanehexol includes crystalline forms which may exist as polymorphs. Solvates of the compounds formed with water or common organic solvents are also intended to be encompassed within the term. In addition, hydrate forms of the compounds and their salts are encompassed within this invention. Further prodrugs of compounds of cyclohexanehexols are encompassed within the term.

The term “solvate” means a physical association of a compound with one or more solvent molecules or a complex of variable stoichiometry formed by a solute (for example, a cyclohexanehexol disclosed herein) and a solvent, for example, water, ethanol, or acetic acid. This physical association may involve varying degrees of ionic and covalent bonding, including hydrogen bonding. In certain instances, the solvate will be capable of isolation, for example, when one or more solvent molecules are incorporated in the crystal lattice of the crystalline solid. In general, the solvents selected do not interfere with the biological activity of the solute. Solvates encompass both solution-phase and isolatable solvates. Representative solvates include hydrates, ethanolates, methanolates, and the like. Dehydrate, co-crystals, anhydrous, or amorphous forms of the cyclohexanhexol compounds are also contemplated herein. The term “hydrate” means a solvate wherein the solvent molecule(s) is/are H₂O, including, mono-, di-, and various poly-hydrates thereof Solvates can be formed using various methods known in the art.

Crystalline compounds of a cyclohexanehexol disclosed herein can be in the form of a free base, a salt, or a co-crystal. Free base compounds can be crystallized in the presence of an appropriate solvent in order to form a solvate. Acid salt compounds of cyclohexanehexols (e.g. HCl, HBr, benzoic acid) can also be used in the preparation of solvates. For example, solvates can be formed by the use of acetic acid or ethyl acetate. The solvate molecules can form crystal structures via hydrogen bonding, van der Waals forces, or dispersion forces, or a combination of any two or all three forces.

The amount of solvent used to make solvates can be determined by routine testing. For example, a monohydrate of a cyclohexanehexol would have about 1 equivalent of solvent (H₂O) for each equivalent of a cyclohexanehexol. However, more or less solvent may be used depending on the choice of solvate desired.

Cyclohexanehexols for use in the present invention may be amorphous or may have different crystalline polymorphs, possibly existing in different solvation or hydration states. By varying the form of a drug, it is possible to vary the physical properties thereof For example, crystalline polymorphs typically have different solubilities from one another, such that a more thermodynamically stable polymorph is less soluble than a less thermodynamically stable polymorph. Pharmaceutical polymorphs can also differ in properties such as shelf-life, bioavailability, morphology, vapor pressure, density, color, and compressibility.

The term “prodrug” means a covalently-bonded derivative or carrier of the parent compound or active drug substance which undergoes at least some biotransformation prior to exhibiting its pharmacological effect(s). In general, such prodrugs have metabolically cleavable groups and are rapidly transformed in vivo to yield the parent compound, for example, by hydrolysis in blood, and generally include esters and amide analogs of the parent compounds. The prodrug is formulated with the objectives of improved chemical stability, improved patient acceptance and compliance, improved bioavailability, prolonged duration of action, improved organ selectivity, improved formulation (e.g., increased hydrosolubility), and/or decreased side effects (e.g., toxicity). In general, prodrugs themselves have weak or no biological activity and are stable under ordinary conditions. Prodrugs can be readily prepared from the parent compounds using methods known in the art, such as those described, for example, in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309 396; Burger's Medicinal Chemistry and Drug Discovery, 5th Ed., M. Wolff (ed.), John Wiley & Sons, 1995, particularly Vol. 1 and pp. 172 178 and pp. 949 982; Pro-Drugs as Novel Delivery Systems, T. Higuchi and V. Stella (eds.), Am. Chem. Soc., 1975; and Bioreversible Carriers in Drug Design, E. B. Roche (ed.), Elsevier, 1987.

Examples of prodrugs include, but are not limited to esters (e.g., acetate, formate, and benzoate derivatives), carbamates (e.g. N,N-dimethylaminocarbonyl) of hydroxy functional groups on compounds of the present invention, and the like.

In general, all physical forms of cyclohexanehexols are intended to be within the scope of the present invention.

A cyclohexanehexol includes a compound with the base structure of the formula I, in particular a substantially pure, compound of the formula I.

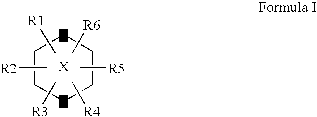

wherein X is a cyclohexane, in particular a myo-, scyllo, epi-, chiro, or allo-inositol radical, wherein one or more of R¹, R², R³, R⁴, R⁵, and R⁶ are independently hydroxyl, alkyl, alkenyl, alkynyl, alkylene, alkenylene, alkoxy, alkenyloxy, cycloalkyl, cycloalkenyl, cycloalkoxy, cycloalkynyl, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, and a pharmaceutically acceptable salt, isomer, solvate, or prodrug thereof. In aspects of the invention, four or five or all of R¹, R², R³, R⁴, R⁵, and/or R⁶ are hydroxyl. In particular aspects of the invention, a cyclohexanehexol of the formula I is used wherein X is a radical of scyllo-inositol or epi-inositol.

Aspects of the invention use classes of cyclohexanehexols of the formula II, in particular isolated and pure, in particular substantially pure, compounds of the formula II:

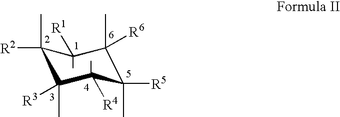

wherein R¹, R², R³, R⁴, R⁵, and R⁶ are hydroxyl, or one or more of R¹, R², R³, R⁴, R⁵, and/or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfinyl, sulfonate, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide and the other of R¹, R², R³, R⁴, R⁵, and/or R⁶ are hydroxyl, or a pharmaceutically acceptable salt thereof.

In aspects of the invention, the cyclohexanehexol is a substantially pure, compound of the formula I or II as defined herein with the proviso that when (a) one of R¹, R², R³, R⁴, R⁵, and/or R⁶ are alkyl or fluorine no more than four of the other of R¹, R², R³, R⁴, R⁵, and/or R⁶ are hydroxyl, (b) one of R¹, R², R³, R⁴, R⁵, and/or R⁶ is amino or azide no more than four of R¹, R², R³, R⁴, R⁵, and R⁶ are hydroxyl, (c) two of R¹, R², R³, R⁴, R⁵, more than three of R¹, R², R³, R⁴, R⁵, and R⁶ are hydroxyl, and (d) three of R¹, R², R³, R⁴, R⁵, and/or R⁶ are amino, carboxyl, carbamyl, sulfonyl, isoxasolyl, imidazolyl, or thiazolyl, the other of R¹, R², R³, R⁴, R⁵, and/or R⁶ cannot all be hydroxyl.

In aspects of the invention, the cyclohexanehexol is a substantially pure, compound of the formula III,

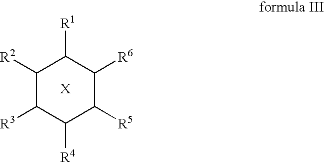

wherein X is a cyclohexane ring, where R¹, R², R³, R⁴, R⁵, and R⁶ are hydroxyl, or at least one of R¹, R², R³, R⁴, R⁵, and R⁶ is independently selected from hydrogen, C₁-C₆ alkyl, C₂-C₆ alkenyl, C₂-C₆ alkynyl, C₁-C₆alkoxy, C₂-C₆ alkenyloxy, C₃-C₁₀ cycloalkyl, C₄-C₁₀cycloalkenyl, C₃-C ₁₀cycloalkoxy, C₆-C₁₀aryl, C₆-C₁₀aryloxy, C₆-C₁₀aryl-C₁-C₃alkoxy, C₆-C₁₀aroyl, C₆-C₁₀heteroaryl, C₃-C₁₀heterocyclic, C₁-C₆acyl, C₁-C₆acyloxy, —NH₂, —NHR⁷R⁸, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀cycloalkyl, C₄-C₁₀cycloalkenyl, C₆-C₁₀aryl, C₆-C₁₀ aryl C₁-C₃alkyl, C₆-C₁₀ heteroaryl and C₃-C₁₀heterocyclic, and at least one of the remainder of R¹, R², R³, R⁴, R⁵, or R⁶ is hydroxyl; or a pharmaceutically acceptable salt thereof In particular aspects, the invention utilizes isomers of the compound of the formula III, more particularly scyllo- or epi- isomers.

In aspects of the invention, the cyclohexanehexol is a substantially pure, compound of the formula IV,

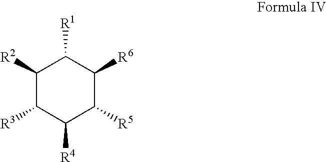

wherein R¹, R², R³, R⁴, R⁵, and R⁶ are defined as for formula III, or a pharmaceutically acceptable salt thereof.

The terms used herein for radicals including “alkyl”, “alkoxy”, “alkenyl”, “alkynyl”, “hydroxyl” etc, refer to both unsubstituted and substituted radicals. The term “substituted,” as used herein, means that any one or more moiety on a designated atom (e.g., hydroxyl) is replaced with a selected group provided that the designated atom's normal valency is not exceeded, and that the substitution results in a stable compound. Combinations of substituents and/or radicals are permissible only if such combinations result in stable compounds. “Stable compound” refers to a compound that is sufficiently robust to survive isolation to a useful degree of purity from a reaction mixture, and formulation into an efficacious therapeutic agent.

“Alkyl”, either alone or within other terms such as “arylalkyl” means a monovalent, saturated hydrocarbon radical which may be a straight chain (i.e., linear) or a branched chain. In certain aspects of the invention, an alkyl radical comprises from about 1 to 24 or 1 to 20 carbon atoms, preferably from about 1 to 10, 1 to 8, 3 to 8, 1 to 6, or 1 to 3 carbon atoms. Examples of alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-octyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof In certain embodiments of the invention an alkyl radical is a C₁-C₆ lower alkyl comprising or selected from the group consisting of methyl, ethyl, n-propyl, n-butyl, n-pentyl, n-hexyl, isopropyl, isobutyl, isopentyl, amyl, tributyl, sec-butyl, tert-butyl, tert-pentyl, and n-hexyl. An alkyl radical may be optionally substituted with substituents at positions that do not significantly interfere with the preparation of the cyclohexanehexols and do not significantly reduce the efficacy of the compounds. An alkyl radical may be optionally substituted. In certain aspects, an alkyl radical is substituted with one to five substituents including halo, lower alkoxy, haloalkoxy, alkylalkoxy, haloalkoxyalkyl, hydroxyl, cyano, nitro, thio, amino, substituted amino, carboxyl, sulfonyl, sulfenyl, sulfinyl, sulfate, sulfoxide, substituted carboxyl, halogenated lower alkyl (e.g. CF₃), halogenated lower alkoxy, hydroxycarbonyl, lower alkoxycarbonyl, lower alkylcarbonyloxy, lower alkylcarbonylamino, aryl (e.g., phenylmethyl (i.e. benzyl)), heteroaryl (e.g., pyridyl), and heterocyclic (e.g., piperidinyl, morpholinyl).

In aspects of the invention, “substituted alkyl” refers to an alkyl group substituted by, for example, one to five substituents, and preferably 1 to 3 substituents, such as alkyl, alkoxy, oxo, alkanoyl, aryl, aralkyl, aryloxy, alkanoyloxy, cycloalkyl, acyl, amino, hydroxyamino, alkylamino, arylamino, alkoxyamino, aralkylamino, cyano, halogen, hydroxyl, carboxyl, carbamyl, carboxylalkyl, keto, thioketo, thiol, alkylthiol, arylthio, aralkylthio, sulfonamide, thioalkoxy, and nitro.

The term “alkenyl” refers to an unsaturated, acyclic branched or straight-chain hydrocarbon radical comprising at least one double bond. Alkenyl radicals may contain from about 2 to 24 or 2 to 10 carbon atoms, preferably from about 3 to 8 carbon atoms and more preferably about 3 to 6 or 2 to 6 carbon atoms. Examples of suitable alkenyl radicals include ethenyl, propenyl such as prop-1-en-1-yl, prop-1-en-2-yl, prop-2-en-1-yl (allyl), prop-2-en-2-yl, buten-1-yl, but-1-en-2-yl, 2-methyl-prop-1-en-1-yl, but-2-en-1-yl, but-2-en-2-yl, buta-1,3-dien-1-yl, buta-1,3-dien-2-yl, hexen-1-yl, 3-hydroxyhexen-1-yl, hepten-1-yl, and octen-1-yl, and the like. Preferred alkenyl groups include ethenyl (—CH═CH₂), n-propenyl (—CH₂CH═CH₂), iso-propenyl (—C(CH₃)═CH₂), and the like. An alkenyl radical may be optionally substituted similar to alkyl.

In aspects of the invention, “substituted alkenyl” refers to an alkenyl group substituted by, for example, one to three substituents, preferably one to two substituents, such as alkyl, alkoxy, haloalkoxy, alkylalkoxy, haloalkoxyalkyl, alkanoyl, alkanoyloxy, cycloalkyl, cycloalkoxy, acyl, acylamino, acyloxy, amino, alkylamino, alkanoylamino, aminoacyl, aminoacyloxy, cyano, halogen, hydroxyl, carboxyl, carboxylalkyl, carbamyl, keto, thioketo, thiol, alkylthio, sulfonyl, sulfonamido, thioalkoxy, aryl, nitro, and the like.

The term “alkynyl” refers to an unsaturated, branched or straight-chain hydrocarbon radical comprising one or more triple bonds. Alkynyl radicals may contain about 1 to 20, 1 to 15, or 2-10 carbon atoms, preferably about 3 to 8 carbon atoms and more preferably about 3 to 6 carbon atoms.

In aspects of the invention, “alkynyl” refers to straight or branched chain hydrocarbon groups of 2 to 6 carbon atoms having one to four triple bonds. Examples of suitable alkynyl radicals include ethynyl, propynyls, such as prop-1-yn-1-yl, prop-2-yn-1-yl, butynyls such as but-1-yn-1-yl, but-1-yn-3-yl, and but-3-yn-1-yl, pentynyls such as pentyn-1-yl, pentyn-2-yl, and 4-methoxypentyn-2-yl, and 3-methylbutyn-1-yl, hexynyls such as hexyn-1-yl, hexyn-2-yl, and hexyn-3-yl, and 3,3-dimethylbutyn-1-yl radicals and the like. This radical may be optionally substituted similar to alkyl. The term “cycloalkynyl” refers to cyclic alkynyl groups.

In aspects of the invention, “substituted alkynyl” refers to an alkynyl group substituted by, for example, a substituent, such as, alkyl, alkoxy, alkanoyl, alkanoyloxy, cycloalkyl, cycloalkoxy, acyl, acylamino, acyloxy, amino, alkylamino, alkanoylamino, aminoacyl, aminoacyloxy, cyano, halogen, hydroxyl, carboxyl, carboxylalkyl, carbamyl, keto, thioketo, thiol, alkylthio, sulfonyl, sulfonamido, thioalkoxy, aryl, nitro, and the like.

The term “alkylene” refers to a linear or branched radical having from about 1 to 10, 1 to 8, 1 to 6, or 2 to 6 carbon atoms and having attachment points for two or more covalent bonds. Examples of such radicals are methylene, ethylene, ethylidene, methylethylene, and isopropylidene.

The term “alkenylene” refers to a linear or branched radical having from about 2 to 10, 2 to 8 or 2 to 6 carbon atoms, at least one double bond, and having attachment points for two or more covalent bonds. Examples of such radicals are 1,1-vinylidene (CH₂═C), 1,2-vinylidene (—CH═CH—), and 1,4-butadienyl (—CH═CH—CH═CH—).

As used herein, “halogen” or “halo” refers to fluoro, chloro, bromo and iodo, especially fluoro or chloro.

The term “hydroxyl” or “hydroxy” refers to a single —OH group.

The term “cyano” refers to a carbon radical having three of four covalent bonds shared by a nitrogen atom, in particular —CN.

The term “alkoxy” refers to a linear or branched oxy-containing radical having an alkyl portion of one to about ten carbon atoms, which may be substituted. Particular alkoxy radicals are “lower alkoxy” radicals having about 1 to 6, 1 to 4 or 1 to 3 carbon atoms. An alkoxy having about 1-6 carbon atoms includes a C₁-C₆ alkyl-O— radical wherein C₁-C₆ alkyl has the meaning set out herein. Illustrative examples of alkoxy radicals include without limitation methoxy, ethoxy, propoxy, butoxy, isopropoxy and tert-butoxy. An “alkoxy” radical may optionally be further substituted with one or more substitutents disclosed herein including alkyl atoms (in particular lower alkyl) to provide “alkylalkoxy” radicals; halo atoms, such as fluoro, chloro or bromo, to provide “haloalkoxy” radicals (e.g. fluoromethoxy, chloromethoxy, trifluoromethoxy, difluoromethoxy, trifluoroethoxy, fluoroethoxy, tetrafluoroethoxy, pentafluoroethoxy, and fluoropropoxy) and “haloalkoxyalkyl” radicals (e.g. fluoromethoxymethyl, chloromethoxyethyl, trifluoromethoxymethyl, difluoromethoxyethyl, and trifluoroethoxymethyl).

The term “acyl”, alone or in combination, means a carbonyl or thiocarbonyl group bonded to a radical selected from, for example, optionally substituted, hydrido, alkyl (e.g. haloalkyl), alkenyl, alkynyl, alkoxy (“acyloxy” including acetyloxy, butyryloxy, iso-valeryloxy, phenylacetyloxy, benzoyloxy, p-methoxybenzoyloxy, and substituted acyloxy such as alkoxyalkyl and haloalkoxy), aryl, halo, heterocyclyl, heteroaryl, sulfinyl (e.g. alkylsulfinylalkyl), sulfonyl (e.g. alkylsulfonylalkyl), cycloalkyl, cycloalkenyl, thioalkyl, thioaryl, amino (e.g., alkylamino or dialkylamino), and aralkoxy. Illustrative examples of “acyl” radicals are formyl, acetyl, 2-chloroacetyl, 2-bromacetyl, benzoyl, trifluoroacetyl, phthaloyl, malonyl, nicotinyl, and the like.

In aspects of the invention, “acyl” refers to a group —C(O)R⁹, where R⁹ is hydrogen, alkyl, cycloalkyl, cycloheteroalkyl, aryl, arylalkyl, heteroalkyl, heteroaryl, and heteroarylalkyl. Examples include, but are not limited to formyl, acetyl, cyclohexylcarbonyl, cyclohexylmethylcarbonyl, benzoyl, benzylcarbonyl and the like.

The term “cycloalkyl” refers to radicals having from about 3 to16 or 3 to 15 carbon atoms and containing one, two, three, or four rings wherein such rings may be attached in a pendant manner or may be fused. In aspects of the invention, “cycloalkyl” refers to an optionally substituted, saturated hydrocarbon ring system containing 1 to 2 rings and 3 to 7 carbons per ring which may be further fused with an unsaturated C₃-C₇ carbocylic ring. Examples of cycloalkyl groups include single ring structures such as cyclopropyl, cyclobutyl, cyclopentyl, cyclohexyl, cycloheptyl, cyclooctyl, cyclononyl, cyclodecyl, cyclododecyl, and the like, or multiple ring structures such as adamantanyl, and the like. In certain aspects of the invention the cycloalkyl radicals are “lower cycloalkyl” radicals having from about 3 to 10, 3 to 8, 3 to 6, or 3 to 4 carbon atoms, in particular cyclopropyl, cyclobutyl, cyclopentyl, cyclohexyl and cycloheptyl. The term “cycloalkyl” also embraces radicals where cycloalkyl radicals are fused with aryl radicals or heterocyclyl radicals. A cycloalkyl radical may be optionally substituted.

In aspects of the invention, “substituted cycloalkyl” refers to cycloalkyl groups having from 1 to 5 (in particular 1 to 3) substituents including without limitation alkyl, alkenyl, alkoxy, cycloalkyl, substituted cycloalkyl, acyl, acylamino, acyloxy, amino, aminoacyl, aminoacyloxy, oxyacylamino, cyano, halogen, hydroxyl, carboxyl, carboxylalkyl, keto, thioketo, thiol, thioalkoxy, aryl, aryloxy, heteroaryl, heteroaryloxy, hydroxyamino, alkoxyamino, and nitro.

The term “cycloalkenyl” refers to radicals comprising about 2 to 16, 4 to 16, 2 to 15, 2 to 10, 4 to 10, 3 to 8, 3 to 6, or 4 to 6 carbon atoms, one or more carbon-carbon double bonds, and one, two, three, or four rings wherein such rings may be attached in a pendant manner or may be fused. In certain aspects of the invention the cycloalkenyl radicals are “lower cycloalkenyl” radicals having three to seven carbon atoms, in particular cyclobutenyl, cyclopentenyl, cyclohexenyl and cycloheptenyl. A cycloalkenyl radical may be optionally substituted with groups as disclosed herein.

The term “cycloalkoxy” refers to cycloalkyl radicals (in particular, cycloalkyl radicals having 3 to 15, 3 to 8 or 3 to 6 carbon atoms) attached to an oxy radical. Examples of cycloalkoxy radicals include cyclohexoxy and cyclopentoxy. A cycloalkoxy radical may be optionally substituted with groups as disclosed herein.

The term “aryl”, alone or in combination, refers to a carbocyclic aromatic system containing one, two or three rings wherein such rings may be attached together in a pendant manner or may be fused. The term “fused” means that a second ring is present (i.e, attached or formed) by having two adjacent atoms in common or shared with the first ring. In aspects of the invention an aryl radical comprises 4 to 24 carbon atoms, in particular 4 to 10, 4 to 8, or 4 to 6 carbon atoms. The term “aryl” includes without limitation aromatic radicals such as phenyl, naphthyl, indenyl, benzocyclooctenyl, benzocycloheptenyl, pentalenyl, azulenyl, tetrahydronaphthyl, indanyl, biphenyl, diphenyl, acephthylenyl, fluorenyl, phenalenyl, phenanthrenyl, and anthracenyl, preferably phenyl. An aryl radical may be optionally subsitituted (i.e., “substituted aryl”) with, for example, one to four substituents such as alkyl, substituted alkyl, alkenyl, substituted alkenyl, alkynyl, substituted alkynyl, aryl, substituted aryl, aralkyl, halo, trifluoromethoxy, trifluoromethyl, hydroxy, alkoxy, alkanoyl, alkanoyloxy, aryloxy, aralkyloxy, amino, alkylamino, arylamino, aralkylamino, dialkylamino, alkanoylamino, thiol, alkylthio, ureido, nitro, cyano, carboxy, carboxyalkyl, carbamyl, alkoxycarbonyl, alkylthiono, arylthiono, arylsulfonylamine, sulfonic acid, alkysulfonyl, sulfonamido, aryloxy and the like. A substituent may be further substituted by hydroxy, halo, alkyl, alkoxy, alkenyl, alkynyl, aryl or aralkyl. In aspects of the invention an aryl radical is substituted with hydroxyl, alkyl, carbonyl, carboxyl, thiol, amino, and/or halo. The term “aralkyl” refers to an aryl or a substituted aryl group bonded directly through an alkyl group, such as benzyl. Other particular examples of substituted aryl radicals include chlorobenyzl, and amino benzyl.

The term “aryloxy” refers to aryl radicals, as defined above, attached to an oxygen atom. Exemplary aryloxy groups include napthyloxy, quinolyloxy, isoquinolizinyloxy, and the like.

The term “arylalkoxy” as used herein, refers to an aryl group attached to an alkoxy group. Representative examples of arylalkoxy include, but are not limited to, 2-phenylethoxy, 3-naphth-2-ylpropoxy, and 5-phenylpentyloxy.

The term “aroyl” refers to aryl radicals, as defined above, attached to a carbonyl radical as defined herein, including without limitation benzoyl and toluoyl. An aroyl radical may be optionally substituted with groups as disclosed herein.

The term “heteroaryl” refers to fully unsaturated heteroatom-containing ring-shaped aromatic radicals having from 3 to 15, 3 to 10, 5 to 15, 5 to 10, or 5 to 8 ring members selected from carbon, nitrogen, sulfur and oxygen, wherein at least one ring atom is a heteroatom. A heteroaryl radical may contain one, two or three rings and the rings may be attached in a pendant manner or may be fused. Examples of “heteroaryl” radicals, include without limitation, an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyrimidyl, pyrazinyl, pyridazinyl, triazolyl, tetrazolyl and the like; an unsaturated condensed heterocyclic group containing 1 to 5 nitrogen atoms, in particular, indolyl, isoindolyl, indolizinyl, benzimidazolyl, quinolyl, isoquinolyl, indazolyl, benzotriazolyl, tetrazolopyridazinyl and the like; an unsaturated 3 to 6-membered heteromonocyclic group containing an oxygen atom, in particular, 2-furyl, 3-furyl, and the like; an unsaturated 5 to 6-membered heteromonocyclic group containing a sulfur atom, in particular, 2-thienyl, 3-thienyl, and the like; unsaturated 5 to 6-membered heteromonocyclic group containing 1 to 2 oxygen atoms and 1 to 3 nitrogen atoms, in particular, oxazolyl, isoxazolyl, and oxadiazolyl; an unsaturated condensed heterocyclic group containing 1 to 2 oxygen atoms and 1 to 3 nitrogen atoms, in particular benzoxazolyl, benzoxadiazolyl and the like; an unsaturated 5 to 6-membered heteromonocyclic group containing 1 to 2 sulfur atoms and 1 to 3 nitrogen atoms, for example, thiazolyl, thiadiazolyl and the like; an unsaturated condensed heterocyclic group containing 1 to 2 sulfur atoms and 1 to 3 nitrogen atoms such as benzothiazolyl, benzothiadiazolyl and the like. The term also includes radicals where heterocyclic radicals are fused with aryl radicals, in particular bicyclic radicals such as benzofuran, benzothiophene, and the like. A heteroaryl radical may be optionally substituted with groups as disclosed herein.

The term “heterocyclic” refers to saturated and partially saturated heteroatom-containing ring-shaped radicals having from about 3 to 15, 3 to 10, 5 to 15, 5 to 10, or 3 to 8 ring members selected from carbon, nitrogen, sulfur and oxygen, wherein at least one ring atom is a heteroatom. A heterocylic radical may contain one, two or three rings wherein such rings may be attached in a pendant manner or may be fused. Examples of saturated heterocyclic radicals include without limitiation a saturated 3 to 6-membered heteromonocylic group containing 1 to 4 nitrogen atoms [e.g. pyrrolidinyl, imidazolidinyl, piperidinyl, and piperazinyl]; a saturated 3 to 6-membered heteromonocyclic group containing 1 to 2 oxygen atoms and 1 to 3 nitrogen atoms [e.g. morpholinyl]; and, a saturated 3 to 6-membered heteromonocyclic group containing 1 to 2 sulfur atoms and 1 to 3 nitrogen atoms [e.g., thiazolidinyl] etc. Examples of partially saturated heterocyclyl radicals include without limitation dihydrothiophene, dihydropyran, dihydrofuran and dihydrothiazole. Illustrative heterocyclic radicals include without limitation 2-pyrrolinyl, 3-pyrrolinyl, pyrrolindinyl, 1,3-dioxolanyl, 2H-pyranyl, 4H-pyranyl, piperidinyl, 1,4-dioxanyl, morpholinyl, 1,4-dithianyl, thiomorpholinyl, and the like.

The term “sulfate”, used alone or linked to other terms, is art recognized and includes a group that can be represented by the formula:

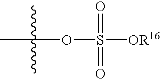

wherein R¹⁶ is an electron pair, hydrogen, alkyl, alkenyl, alkynyl, cycloalkyl, cycloalkenyl, cycloalkynyl, aryl, heterocyclic, carbohydrate, peptide or peptide derivative.

The term “sulfonyl”, used alone or linked to other terms such as alkylsulfonyl or arylsulfonyl, refers to the divalent radicals —SO₂—. In aspects of the invention where one or more of R¹, R³, R⁴, R⁵, or R⁶, is a sulfonyl group, the sulfonyl group may be attached to a substituted or unsubstituted alkyl, alkenyl, alkynyl, aryl, cycloalkyl, cycloalkenyl, cycloalkynyl, or heterocyclic group, carbohydrate, peptide, or peptide derivative .

The term “sulfonate” is art recognized and includes a group represented by the formula:

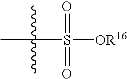

wherein R^{16′} is an electron pair, hydrogen, alkyl, cycloalkyl, aryl, alkenyl, alkynyl, cycloalkenyl, cycloalkynyl, heterocyclic, carbohydrate, peptide, or peptide derivative

Examples of sulfonated alkyl groups include ethyl sulfuric acid, ethanesulfonic acid, 2-aminoethan-1-ol sulfuric acid, 1-propanesulfonic acid, 2-propanesulfonic acid, 1,2-diethanedisulfonic acid, 1,2-ethanediol disulfuric acid, 1,3-propanedisulfonic acid, 1-propanol sulfuric acid, 1,3-propanediol disulfuric acid, 1-butanesulfonic acid, 1,4-butanediol disulfuric acid, 1,2-ethanediol disulfuric acid, 3-amino-1-propanesulfonic acid, 3-hydroxypropanesulfonic acid sulfate, 1,4-butanesulfonic acid, 1,4-butanediol monosulfuric acid, 1-pentanesulfonic acid, 1,5-pentanedisulfonic acid, 1,5-pentanediol sulfuric acid, 4-heptanesulfonic acid, 1,3,5-heptanetriol trisulfate, 2-hydroxymethyl-1,3-propanediol trisulfate, 2-hydroxymethyl-2-methyl-1,3-propanediol trisulfate, 1,3,5,7-heptanetetraol tetrasulfate, 1,3,5,7,9-nonane pentasulfate, 1-decanesulfonic acid, and pharmaceutically acceptable salts thereof.

Examples of cycloalkyl sulfonated groups include 1,3-cyclohexanediol disulfate, and 1,3,5-heptanetriol trisulfate.

Examples of aryl sulfonated groups include 1,3-benzenedisulfonic acid, 2,5-dimethoxy-1,4-benzenedisulfonic acid, 4-amino-3-hydroxy-1-naphthalenesulfonic acid, 3,4-diamino-1-naphthalenesulfonic acid, and pharmaceutically acceptable salts thereof.

Examples of heterocyclic sulfonated compounds include 3-(N-morpholino)propanesulfonic acid and tetrahydrothiophene-1,1-dioxide-3,4-disulfonic acid, and pharmaceutically acceptable salts thereof.

Examples of sulfonated carbohydrates are sucrose octasulfonate, 5-deoxy-1,2-O-isopropylidene-α-D-xylofuranose-5-sulfonic acid or an alkali earth metal salt thereof, methyl-α-D-glucopyranoside 2,3-disulfate, methyl 4,-O-benzylidene-α-D-glucopyranoside 2,3-disulfate, 2,3,4,3′,4′-sucrose pentasulfate, 1,3:4,6-di-O-benzylidene-D-mannitol 2,5-disulfate, D-mannitol 2,5-disulfate, 2,5-di-O-benzyl-D-mannitol tetrasulfate, and pharmaceutically acceptable salts thereof.

The term “sulfinyl”, used alone or linked to other terms such as alkylsulfinyl (i.e. —S(O)-alkyl) or arylsulfinyl, refers to the divalent radicals —S(O)—.

The term “sulfoxide” refers to the radical —S═O.

The term “amino”, alone or in combination, refers to a radical where a nitrogen atom (N) is bonded to three substituents being any combination of hydrogen, hydroxyl, alkyl, cycloalkyl, alkenyl, alkynyl, aryl or silyl with the general chemical formula —NR¹⁰R¹¹ where R¹⁰ and R¹¹ can be any combination of hydrogen, hydroxyl, alkyl, cycloalkyl, alkenyl, alkynyl, aryl, silyl, heteroaryl, or heterocyclic which may or may not be substituted. Optionally one substituent on the nitrogen atom may be a hydroxyl group (—OH) to provide an amine known as a hydroxylamine. Illustrative examples of amino groups are amino (—NH₂), alkylamino, acylamino, cycloamino, acycloalkylamino, arylamino, arylalkylamino, and lower alkylsilylamino, in particular methylamino, ethylamino, dimethylamino, 2-propylamino, butylamino, isobutylamino, cyclopropylamino, benzylamino, allylamino, hydroxylamino, cyclohexylamino, piperidine, benzylamino, diphenylmethylamino, tritylamino, trimethylsilylamino, and dimethyl-tert.-butylsilylamino.

The term “thiol” means —SH.

The term “sulfenyl” refers to the radical —SR¹² wherein R¹² is not hydrogen. R¹² may be alkyl, alkenyl, alkynyl, cycloalkyl, aryl, silyl, heterocyclic, heteroaryl, carbonyl, or carboxyl.

The term “thioalkyl”, alone or in combination, refers to a chemical functional group where a sulfur atom (S) is bonded to an alkyl, which may be substituted. Examples of thioalkyl groups are thiomethyl, thioethyl, and thiopropyl.

The term “thioaryl”, alone or in combination, refers to a chemical functional group where a sulfur atom (S) is bonded to an aryl group with the general chemical formula —SR¹³ where R¹³ is an aryl group which may be substituted. Illustrative examples of thioaryl groups and substituted thioaryl groups are thiophenyl, para-chlorothiophenyl, thiobenzyl, 4-methoxy-thiophenyl, 4-nitro-thiophenyl, and para-nitrothiobenzyl.

The term “thioalkoxy”, alone or in combination, refers to a chemical functional group where a sulfur atom (S) is bonded to an alkoxy group with the general chemical formula —SR¹⁵ where R¹⁵ is an alkoxy group which may be substituted. In aspects of the invention a “thioalkoxy group” has 1-6 carbon atoms and refers to a —S—(O)—C₁-C₆ alkyl group wherein C₁-C₆ alkyl have the meaning as defined above. Illustrative examples of a straight or branched thioalkoxy group or radical having from 1 to 6 carbon atoms, also known as a C₁-C₆ thioalkoxy, include thiomethoxy and thioethoxy.

The term “carbonyl” refers to a carbon radical having two of the four covalent bonds shared with an oxygen atom.

The term “carboxyl” ,alone or in combination, refers to —C(O)OR¹⁴— wherein R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted. In aspects of the invention, the carboxyl groups are in an esterified form and may contain as an esterifying group lower alkyl groups. In particular aspects of the invention, —C(O)OR¹⁴ provides an ester or an amino acid derivative. An esterified form is also particularly referred to herein as a “carboxylic ester”. In aspects of the invention a “carboxyl” may be substituted, in particular substituted with alkyl which is optionally substituted with one or more of amino, amine, halo, alkylamino, aryl, carboxyl, or a heterocyclic. In particular aspects of the invention, the carboxyl group is methoxycarbonyl, butoxycarbonyl, tert.alkoxycarbonyl such as tert.butoxycarbonyl, arylmethyoxycarbonyl having one or two aryl radicals including without limitation phenyl optionally substituted by, for example, lower alkyl, lower alkoxy, hydroxyl, halo, and/or nitro, such as benzyloxycarbonyl, methoxybenxyloxycarbonyl, diphenylmethoxycarbonyl, 2-bromoethoxycarbonyl, 2-iodoethoxycarbonyltert.butylcarbonyl, 4-nitrobenzyloxycarbonyl, diphenylmethoxy-carbonyl, benzhydroxycarbonyl, di-(4-methoxyphenyl-methoxycarbonyl, 2-bromoethoxycarbonyl, 2-iodoethoxycarbonyl, 2-trimethylsilylethoxycarbonyl, or 2-triphenylsilylethoxycarbonyl. Additional carboxyl groups in esterified form are silyloxycarbonyl groups including organic silyloxycarbonyl. The silicon substituent in such compounds may be substituted with lower alkyl (e.g. methyl), alkoxy (e.g. methoxy), and/or halo (e.g. chlorine). Examples of silicon substituents include trimethylsilyl and dimethyltert.butylsilyl.

The term “carboxamide”, alone or in combination, refers to amino, monoalkylamino, dialkylamino, monocycloalkylamino, alkylcycloalkylamino, and dicycloalkylamino radicals, attached to one of two unshared bonds in a carbonyl group.

The term “nitro” means —NO₂—.

A radical in a cyclohexanehexol may be substituted with one or more substituents apparent to a person skilled in the art including without limitation alkyl, alkenyl, alkynyl, alkanoyl, alkylene, alkenylene, hydroxyalkyl, haloalkyl, haloalkylene, haloalkenyl, alkoxy, alkenyloxy, alkenyloxyalkyl, alkoxyalkyl, aryl, alkylaryl, haloalkoxy, haloalkenyloxy, heterocyclic, heteroaryl, sulfonyl, sulfenyl, alkylsulfonyl, sulfinyl, alkylsulfinyl, aralkyl, heteroaralkyl, cycloalkyl, cycloalkenyl, cycloalkoxy, cycloalkenyloxy, amino, oxy, halo, azido, thio, cyano, hydroxyl, phosphonato, phosphinato, thioalkyl, alkylamino, arylamino, arylsulfonyl, alkylcarbonyl, arylcarbonyl, heteroarylcarbonyl, heteroarylsulfinyl, heteroarylsulfonyl, heteroarylamino, heteroaryloxy, heteroaryloxylalkyl, arylacetamidoyl, aryloxy, aroyl, aralkanoyl, aralkoxy, aryloxyalkyl, haloaryloxyalkyl, heteroaroyl, heteroaralkanoyl, heteroaralkoxy, heteroaralkoxyalkyl, thioaryl, arylthioalkyl, alkoxyalkyl, and acyl groups. In embodiments of the invention, the substituents include alkyl, alkoxy, alkynyl, halo, amino, thio, oxy, and hydroxyl.

While broad definitions of cyclohexanehexols are described herein for use in the present invention, certain compounds of formula I, II, III or IV may be more particularly described.

In embodiments of the invention, the cyclohexanehexol is an isolated, in particular pure, more particularly substantially pure, compound of the formula I, wherein X is a radical of scyllo-inositol, epi-inositol or a configuration isomer thereof, wherein
(a) R¹, R², R³, R⁴, R⁵, and R⁶ are hydroxyl, or
(b) one or more of, two or more of, or three or more of R¹, R², R³, R⁴, R⁵, and/or R⁶ are independently optionally substituted alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide and the other of R¹, R², R³, R⁴, R⁵, and/or R⁶ is a hydroxyl.

In embodiments of the invention, the cyclohexanehexol is an isolated, in particular pure, more particularly, substantially pure, compound of the formula II wherein
(a) R¹, R², R³, R⁴, R⁵, and R⁶ are hydroxyl, or
(b) one or more of, two or more of, or three or more of R¹, R², R³, R⁴, R⁵, and/or R⁶ are independently optionally substituted alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfinyl, sulfonate, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide and the other of R¹, R², R³, R⁴, R⁵, and/or R⁶ is a hydroxyl.

In particular aspects of the invention, a cyclohexanehexol does not include a compound of the formula I or II where (a) when one of R¹, R², R³, R⁴, R⁵ and/or R⁶ are alkyl or fluorine, more than 4 of the other of R¹, R², R³, R⁴, R⁵, and/or R⁶ are hydroxyl, (b) when one of R¹, R², R³, R⁴, R⁵, and/or R⁶ is amino or azide, more than four of R¹, R², R³, R⁴, R⁵, and/or R⁶ are hydroxyl, (c) when two of R¹, R², R³, R⁴, R⁵, and/or R⁶ are amino, more than three of R¹, R², R³, R⁴, R⁵, and/or R⁶ are hydroxyl, and (d) R¹, R², R³, R⁴, R⁵, and/or R⁶ are isopropylidene.

In some aspects of the invention, a cyclohexanehexol is utilized where one or more of R¹, R², R³, R⁴, R⁵, and/or R⁶ are alkyl, alkoxy, or halo, and the other of R¹, R², R³, R⁴, R⁵, and/or R⁶ is hydrogen.

In embodiments of the invention, the cyclohexanehexol is a compound of the formula

I or II where the hydrogen at one or more of positions 1, 2, 3, 4, 5, or 6 of formula I or II is substituted with a radical disclosed herein for R¹, R², R³, R⁴, R⁵, and R⁶, including optionally substituted alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfinyl, sulfonate, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, in particular optionally substituted alkyl, alkenyl, alkoxy, amino, imino, thiol, nitro, cyano, halo, or carboxyl.

In embodiments of the invention, the cyclohexanehexol is a compound of the formula I or II wherein one or more of, two or more of, or three or more of R¹, R², R³, R⁴, R⁵, and/or R⁶ are independently alkenyl, alkynyl, alkylene, alkenylene, alkoxy, alkenyloxy, cycloalkenyl, cycloalkoxy, aryl, aryloxy, arylalkoxy, aroyl, heteroaryl, heterocyclic, acyl, acyloxy, sulfonyl, sulfenyl, sulfinyl, sulfonate, sulfoxide, sulfate, nitro, cyano, isocyanato, thioaryl, thioalkoxy, seleno, silyl, silyloxy, silylthio, Cl, I, Br, carboxyl, carboxylic ester, carbonyl, carbamoyl, or carboxamide and the other of R¹, R², R³, R⁴, R⁵, and/or R⁶ is a hydroxyl.

In embodiments of the invention, the cyclohexanehexol is an isolated, in particular pure, more particularly, substantially pure, compound of the formula I or II wherein one or more of, two or more of, or three or more of R¹, R², R³, R⁴, R⁵, and/or R⁶ are independently C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, C₃-C₈ cycloalkoxy, acyloxy, sulfonyl, sulfenyl, sulfinyl, sulfonate, sulfoxide, sulfate, isocyanato, thioaryl, thioalkoxy, selene, silyl, silyloxy, silythio, aryl, aroyl, aryloxy, arylC₁-C₆alkoxy, acetyl, heteroaryl, heterocyclic, amino, thiol, thioalkyl, thioalkoxy, nitro, cyano, halo (e.g., Cl, I, or Br), carboxyl, carboxylic ester, carbonyl, carbamoyl, or carboxamide and the other of R¹, R², R³, R⁴, R⁵, and/or R⁶ is a hydroxyl. In particular aspects, (a) when one of R¹, R², R³, R⁴, R⁵, and/or R⁶ are alkyl or fluorine no more than 4 of the other of R¹, R², R³, R⁴, R⁵, and/or R⁶ are hydroxyl, (b) when one of R¹, R², R³, R⁴, R⁵, and/or R⁶ is amino no more than four of R¹, R², R³, R⁴, R⁵, and/or R⁶ are hydroxyl, (c) when two of R¹, R², R³, R⁴, R⁵, and/or R⁶ are amino, no more than three of R¹, R², R³, R⁴, R⁵, and R⁶ are hydroxyl, and (d) R¹, R², R³, R⁴, R⁵, and/or R⁶ are not isopropylidene.

In embodiments of the invention, the cyclohexanehexol is a compound of the formula I wherein R² is hydroxyl in an equatorial position, at least one, two, three, or four of R¹, R³, R⁴, R⁵, and/or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfenyl, sulfonyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, in particular C₁-C₆ alkyl, C₃-C₆ alkenyl, C₂-C₆ alkynyl, C₂-C₆ alkylene, C₂-C₈alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br, and the other of R¹, R³, R⁴, R⁵, and/or R⁶ are hydroxyl.

In embodiments of the invention, the cyclohexanehexol is a compound of the formula I wherein R² is hydroxyl in an equatorial position, at least two of R¹, R³, R⁴, R⁵, and/or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br, and the other of R¹, R³, R⁴, R⁵, and/or R⁶ are hydroxyl.

In embodiments of the invention, the cyclohexanehexol is a compound of the formula I or II wherein at least two of R¹, R², R³, R⁴, R⁵, and/or R⁶ are hydroxyl, and one, two, three or four or more of the other of R¹, R², R³, R⁴, R⁵, and/or R⁶ are alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexanehexol is a compound of the formula I or II wherein at least two of R¹, R², R³, R⁴, R⁵, and/or R⁶ are hydroxyl, and two or more of the other of R¹, R², R³, R⁴, R⁵, and/or R⁶ are alkyl, cycloalkyl, alkenyl, cycloalkenyl, alkynyl, alkylene, alkenylene, alkoxy, alkenyloxy, cycloalkoxy, aryl, aryloxy, arylalkoxy, aroyl, heteroaryl, heterocyclic, acyl, or acyloxy, sulfonyl, sulfenyl, sulfinyl, amino, imino, cyano, isocyanato, seleno, silyl, silyloxy, silylthio, thiol, thioalkyl, thioalkoxy, halo, carboxyl, carboxylic ester, carbonyl, carbamoyl, and carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexanehexol is a compound of the formula I or II wherein at least two of R¹, R², R³, R⁴, R⁵, and/or R⁶ are hydroxyl, and three or more of the other of R¹, R², R³, R⁴, R⁵, and/or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, azido, nitro, cyano, isocyanato, halo, seleno, silyl, silyloxy, silylthio, carboxyl, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexanehexol is a compound of the formula I or II wherein at least three of R¹, R², R³, R⁴, R⁵, and/or R⁶ are hydroxyl, and one, two, or three of the other of R¹, R², R³, R⁴, R⁵, and/or R⁶ are alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexanehexol is a compound of the formula I or II wherein at least four of R¹, R², R³, R⁴, R⁵, and/or R⁶ are hydroxyl, and one or two of the other of R¹, R³, R⁴, R⁵, and/or R⁶ are alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfonate, sulfenyl, sulfinyl, amino, imino, azido, thiol, thioalkyl, thioalkoxy, thioaryl, azido, nitro, cyano, isocyanato, halo, seleno, silyl, silyloxy, silylthio, carboxyl, carboxylic ester, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈

In embodiments of the invention, the cyclohexanehexol is a compound of the formula I or II wherein R¹, R², R⁴, R⁵, and R⁶ are hydroxyl, and R³ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, azido, nitro, cyano, isocyanato, halo, seleno, silyl, silyloxy, silylthio, carboxyl, carboxylic ester, carbonyl, carbamoyl, or carboxamide. In embodiments, R³ is selected from the group consisting of alkenyl, alkynyl, alkylene, alkenylene, alkoxy, alkenyloxy, cycloalkyl, cycloalkenyl, cycloalkoxy, aryl, aryloxy, arylalkoxy, aroyl, imino, heteroaryl, heterocyclic, acyl, acyloxy, sulfonyl, sulfenyl, sulfinyl, sulfoxide, sulfate, thioalkoxy, thioaryl, carboxyl, carbonyl, carbamoyl, or carboxamide, in particular alkoxy, sulfonyl, sulfenyl, sulfinyl, sulfoxide, sulfate, thioalkoxy, carboxyl, carbonyl, carbamoyl, or carboxamide. In a particular embodiment, R³ is selected from the group consisting of C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, aryl, aryloxy, arylC₁-C₆alkoxy, acetyl, halo, and carboxylic ester, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexanehexol is a compound of the formula I or II wherein R¹, R³, R⁴, R⁵, and R⁶ are hydroxyl, and R² is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, azido, nitro, cyano, isocyanato, halo, seleno, silyl, silyloxy, silylthio, carboxyl, carboxylic ester, carbonyl, carbamoyl, or carboxamide. In embodiments, R² is selected from the group consisting of C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, aryl, aryloxy, arylC₁-C₆alkoxy, acetyl, halo, and carboxylic ester.

In embodiments of the invention, the cyclohexanehexol is a compound of the formula I, II, III or IV wherein one, two, three, four or five of R¹, R², R³, R⁴, R⁵, and/or R⁶ are each independently:
(a) alkyl with 1 to 24 carbon atoms, in particular 1 to 10 or 1 to 6 carbon atoms;
(b) cycloalkyl with 3 to 16 carbon atoms, in particular 3 to 10 or 3 to 6 carbon atoms;
(c) alkenyl with 2 to 24 carbon atoms, in particular 2 to 10 or 2 to 6 carbon atoms;
(d) cycloalkenyl with 4 to 16 carbon atoms, in particular 4 to 10 or 4 to 6 carbon atoms;
(e) aryl with 4 to 24 carbon atoms, in particular 4 to 10, 4 to 8, or 6 or carbon atoms;
(f) aralkyl, alkaryl, aralkenyl, or alkenylaryl;
(g) heterocyclic group comprising 3 to 10, in particular 3 to 8 or 3 to 6 ring members and at least one atom selected from the group consisting of oxygen, nitrogen, and sulfur;
(h) alkoxy with 1 to 6 carbon atoms or 1 to 3 carbon atoms in particular methoxy, ethoxy, propoxy, butoxy, isopropoxy or tert-butoxy, especially methoxy, or
(i) halo, in particular fluorine, chlorine, or bromine, especially chlorine.

In embodiments of the invention, the cyclohexanehexol is a compound of the formula I, II, III or IV wherein R² is hydroxyl and one, two, three, four or five of R¹, R³, R⁴, R⁵, and/or R⁶ is each independently methyl, ethyl, propyl, butyl, pentyl, hexyl, heptyl, octyl, nonyl, decyl, dodecyl, tetradecyl, pentadecyl, hexadecyl, heptadecyl, octadecyl, eicosyl, docosyl, methoxy, ethoxy, propoxy, butoxy, isopropoxy, tert-butoxy, chloro, cyclopropyl, cyclopentyl, cyclohexyl, vinyl, allyl, propenyl, octadienyl, octenyl, decenyl, dodecenyl, tetradecenyl, hexadecenyl, octadecenyl, octadecadienyl, nonadecenyl, octadecatrienyl, arachidonyl, cyclopentenyl, cycopentadienyl, cyclohexenyl, cyclohexadienyl, phenyl, biphenyl, terphenyl, naphtyl, anthracenyl, phenanthrenyl, pyridyl, furyl, or thiazolyl.

In embodiments of the invention, the cyclohexanehexol is a compound of the formula I, II, III or IV wherein R¹ is hydroxyl and one, two, three, four or five of R², R³, R⁴, R⁵, and/or R⁶ is each independently methyl, ethyl, propyl, butyl, pentyl, hexyl, heptyl, octyl, nonyl, decyl, dodecyl, tetradecyl, pentadecyl, hexadecyl, heptadecyl, octadecyl, eicosyl, docosyl, methoxy, ethoxy, propoxy, butoxy, isopropoxy, tert-butoxy, chloro, cyclopropyl, cyclopentyl, cyclohexyl, vinyl, allyl, propenyl, octadienyl, octenyl, decenyl, dodecenyl, tetradecenyl, hexadecenyl, octadecenyl, octadecadienyl, nonadecenyl, octadecatrienyl, arachidonyl, cyclopentenyl, cycopentadienyl, cyclohexenyl, cyclohexadienyl, phenyl, biphenyl, terphenyl, naphtyl, anthracenyl, phenanthrenyl, pyridyl, furyl, or thiazolyl.

In embodiments of the invention, the cyclohexanehexol is a compound of the formula I, II, III or IV wherein one or two of R¹, R², R³, R⁴, R⁵, and/or R⁶ are carboxyl, carbamyl, sulfonyl, or a heterocyclic comprising a N atom, more particularly N-methylcarbamyl, N-propylcarbamyl, N-cyanocarbamyl, aminosulfonyl, isoxazolyl, imidazolyl, and thiazolyl.

In embodiments of the invention, the cyclohexanehexol is a compound of the formula I, II, III or IV where R² is hydroxyl; and R¹, R³, R⁴, R⁵, and R⁶ are independently selected from C₁-C₆alkyl, C₂-C₆alkenyl, C₂-C₆alkynyl, C₁C₆ alkoxy, C₂-C₆alkenyloxy, C₃-C₁₀cycloalkyl, C₄-C ₁₀cycloalkenyl, C₃-C₁₀cycloalkoxy, C₆-C₁₀aryl, C₆-C₁₀aryloxy, C₆-C₁₀aryl-C₁-C₃alkoxy, C₆-C₁₀aroyl, C₆-Cₘheteroaryl, C₃-C₁₀ heterocyclic, C₁-C₆acyl, C₁-C₆acyloxy, hydroxyl, —NH₂, —NHR⁷, —NR⁷R⁸—, ═NW, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀ cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic; provided that R¹, R2, R³, R⁴, R⁵, and R⁶ are not all hydroxyl.

In embodiments of the invention, the cyclohexanehexol is a compound of the formula I, II, III or IV where R² is hydroxyl; one of R¹, R³, R⁴, R⁵, and R⁶ is hydroxyl; and four of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀ cycloalkyl, C₄-C₁₀cycloalkenyl, C₃-C₁₀cycloalkoxy, C₆-C₁₀aryl, C₆-C₁₀aryloxy, C₆-C₁₀ aryl-C₁-C₃alkoxy, C₆-C₁₀aroyl, C₆-C₁₀ heteroaryl, C₃-C₁₀heterocyclic, C₁-C₆ acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀ heteroaryl and C₃-C₁₀ heterocyclic.

In embodiments of the invention, the cyclohexanehexol is a compound of the formula I, II, III or IV where R² is hydroxyl; two of R¹, R³, R⁴, R⁵, and R⁶ are hydroxyl; and three of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀cycloalkyl, C₄-C₁₀cycloalkenyl, C₃-C₁₀cycloalkoxy, C₆-C₁₀aryl, C₆-C₁₀aryloxy, C₆-C₁₀ aryl-C₁-C₃alkoxy, C₆-C₁₀aroyl, C₆-C₁₀heteroaryl, C₃-C₁₀heterocyclic, C₁-C₆acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic.

In embodiments of the invention, the cyclohexanehexol is a compound of the formula III or IV where R² is hydroxyl; three of R¹, R³, R⁴, R⁵, and R⁶ is hydroxyl; and two of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀ cycloalkyl, C₄-C₁₀cycloalkenyl, C₃-C₁₀cycloalkoxy, C₆-C₁₀aryl, C₆-C₁₀aryloxy, C₆-C₁₀ aryl-C₁-C₃alkoxy, C₆-C₁₀aroyl, C₆-C₁₀ heteroaryl, C₃-C₁₀heterocyclic, C₁-C₆ acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic.

In embodiments of the invention, the cyclohexanehexol is a compound of the formula III or IV where R² is hydroxyl; four of R¹, R³, R⁴, R⁵, and R⁶ are hydroxyl; and one of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀ cycloalkyl, C₄-C₁₀cycloalkenyl, C₃-C₁₀cycloalkoxy, C₆-C₁₀ aryl, C₆-C₁₀aryloxy, C₆-C₁₀ aryl-C₁-C₃alkoxy, C₆-C₁₀aroyl, C₆-C₁₀heteroaryl, C₃-C₁₀heterocyclic, C₁-C₆ acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic.

In embodiments of the invention, the cyclohexanehexol is a compound of the formula III or IV wherein one of R¹, R³, R⁴, R⁵, and R⁶ is C₁-C₆alkyl, C₁-C₆alkoxy, C₁-C₆acyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷R⁸ are as defined above; and no more than four of the remainder of R¹, R², R³, R⁴, R⁵, and R⁶ are hydroxyl.

In embodiments of the invention, the cyclohexanehexol is a compound of the formula III or IV wherein two of R¹, R³, R⁴, R⁵, and R⁶ are C₁-C₆alkyl, C₁-C₆alkoxy, C₁-C₆acyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷R⁸ are as defined above; and no more than three of R¹, R², R³, R⁴, R⁵, and R⁶ are hydroxyl,

In embodiments of the invention, the cyclohexanehexol is a compound of the formula III or IV wherein three of R¹, R³, R⁴, R⁵, and R⁶ are C₁-C₆alky, C₁-C₆alkoxy, C₁-C₆alkyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷R⁸ are as defined above; and no more than two of R¹, R², R³, R⁴, R⁵, and R⁶ are hydroxyl.

In embodiments of the invention, the cyclohexanehexol is a compound of the formula I, II, III or IV wherein one, two, three, four or five of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, alkoxy, acetyl, halo, carboxylic ester, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, the cyclohexanehexol is a compound of the formula I, II, III or IV wherein two of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, alkoxy, acetyl, halo, carboxylic ester, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, the cyclohexanehexol is a compound of the formula I, II, III or IV wherein three of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, alkoxy, acetyl, halo, carboxylic ester, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, the cyclohexanehexol is a compound of the formula I, II, III or IV wherein four of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, alkoxy, acetyl, halo, carboxylic ester, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, the cyclohexanehexol is a compound of the formula I, II, III or IV wherein five of R¹, R², R³, R⁴, R⁵, and/or R⁶ are hydroxyl and the other of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy, which may be substituted with with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, the cyclohexanehexol is a compound of the formula I, II, III or IV wherein one, two, or three of R¹, R², R³, R⁴, R⁵, and/or R⁶ is each independently —OR¹⁷ where R¹⁷ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide or a carbohydrate. In an aspect, wherein one, two, or three of R¹, R², R³, R⁴, R⁵, and/or R⁶ is each independently —OR¹⁷ where R¹⁷ is C₁-C₆ alkyl, most particularly C₁-C₃ alkyl.

In selected cyclohexanehexols of the formula I, II, III or IV, at least one of R¹, R², R³, R⁴, R⁵, and/or R⁶ is —OR²⁰ wherein R²⁰ is —CF₃, CF₃CF₂, CF₃CH₂, CH₂NO₂, CH₂NH₂, C(CH₂)₃, or cyclopropyl.

In embodiments of the invention, the cyclohexanehexol is a compound of the formula I, II, III or IV wherein R¹, R², R³, R⁴, and R⁵ are hydroxyl and R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In a particular embodiment of the invention, R¹, R², R³, R⁴, and R⁵ are hydroxyl and R⁶ is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R¹, R², R³, R⁴, and R⁵ are hydroxyl and R⁶ is methoxy.

In embodiments of the invention, the cyclohexanehexol is a compound of the formula I, II, III or IV wherein R¹, R², R³, R⁴, and R⁶ are hydroxyl and R⁵ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In a particular embodiment of the invention, R¹, R², R³, R⁴, and R⁶ are hydroxyl and R⁵ is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R¹, R², R³, R⁴, and R⁶ are hydroxyl and R⁵ is methoxy.

In embodiments of the invention, the cyclohexanehexol is a compound of the formula I, II, III or IV wherein R¹, R², R³, R⁵, and R⁶ are hydroxyl and R⁴ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R¹, R², R³, R⁵, and R⁶ are hydroxyl and R⁴ is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R¹, R², R³, R⁵, and R⁶ are hydroxyl and R⁴ is methoxy.

In embodiments of the invention, the cyclohexanehexol is a compound of the formula I, II, III or IV wherein R¹, R², R⁴, R⁵, and R⁶ are hydroxyl and R³ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R¹, R², R⁴, R⁵, and R⁶ are hydroxyl and R³ is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R¹, R², R⁴, R⁵, and R⁶ are hydroxyl and R³ is methoxy.

In embodiments of the invention, the cyclohexanehexol is a compound of the formula I, II, III or IV wherein R¹, R³, R⁴, R⁵, and R⁶ are hydroxyl and R² is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R¹, R³, R⁴, R⁵, and R⁶ are hydroxyl and R² is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R¹, R³, R⁴, R⁵, and R⁶ are hydroxyl and R² is methoxy.

In embodiments of the invention, the cyclohexanehexol is a compound of the formula I, II, III or IV wherein R², R³, R⁴, R⁵, and R⁶ are hydroxyl and R¹ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R², R³, R⁴, R⁵, and R⁶ are hydroxyl and R¹ is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R², R³, R⁴, R⁵, and R⁶ are hydroxyl and R¹ is methoxy.

In embodiments of the invention, the cyclohexanehexol is a compound of the formula III or IV, wherein two, three, four or five of R¹, R², R³, R⁴, R⁵, or R⁶ are hydroxyl; at least one of R¹, R², R³, R⁴, R⁵, or R⁶ is optionally substituted alkoxy; and the remainder of R¹, R², R³, R⁴, R⁵, or R⁶ if any are independently selected from C₁-C₆alkyl, C₂-C₆alkenyl, C₂-C₆alkynyl, C₁C₆alkoxy, C₂-C₆alkenyloxy, C₃-C₁₀cycloalkyl, C₁-C₆acyl, C₁-C₆ acyloxy, hydroxyl, —NH₂, —NHR⁷, —NR⁷R⁸—, ═NR⁷, —S(O)₂R⁷, —SH, nitro, cyano, halo, haloalkyl, haloalkoxy, hydroxyalkyl, —CO₂R⁷, oxo, —PO₃H—NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀arylC₁-C₃alkyl, C₆-C₁₀heteroaryl and C₃-C₁₀heterocyclic.

In embodiments of the invention, the cyclohexanehexol is a compound of the formula III or IV, wherein five of R¹, R², R³, R⁴, R⁵, or R⁶ are hydroxyl; and one of R¹, R², R³, R⁴, R⁵, or R⁶ is C₁-C₆alkoxy; for example at least one of R¹, R², R³, R⁴, R⁵, is methoxy.

In embodiments of the invention, the cyclohexanehexol is a compound of the formula IV, wherein two, three, or four of R², R³, R⁴, R⁵, or R⁶ are hydroxyl; R¹ is optionally substituted alkoxy; and the remainder of R², R³, R⁴, R⁵, or R⁶ are independently selected from C₁-C₆alkyl, C₂-C₆alkenyl, C₂-C₆alkynyl, C₁-C₆alkoxy, C₂-C₆alkenyloxy, C₃-C₁₀cycloalkyl, C₁-C₆acyl, C₁-C₆acyloxy, hydroxyl, —NH₂, —NHR⁷, —NR⁷R⁸—, ═NR⁷, —S(O)₂R⁷, —SH, nitro, cyano, halo, haloalkyl, haloalkoxy, hydroxyalkyl, —CO₂R⁷, oxo, —PO₃H—NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀ heteroaryl and C₃-C₁₀ heterocyclic.

In embodiments of the invention, the cyclohexanehexol is a compound of the formula IV, wherein R¹ is C₁-C₆ alkoxy; and R², R³, R⁴, R⁵, and R⁶ are hydroxyl; for example R¹ is methoxy.

In embodiments of the invention, the cyclohexanehexol is a compound of the formula I, II, III or IV wherein five of R¹, R², R³, R⁴, R⁵, and/or R⁶ are hydroxyl and the other of R¹, R², R³, R⁴, R⁵, and/or R⁶ is substituted alkoxy, in particular alkoxy having about 1-6 carbon atoms, more particularly methoxy, ethoxy, propoxy, butoxy, isopropoxy and tert-butoxy, substituted with alkyl, in particular C₁-C₆ alkyl, more particularly C₁-C₃ alkyl.

In embodiments of the invention, the cyclohexanehexol is a compound of the formula I, II, III or IV wherein five of R¹, R², R³, R⁴, R⁵, and/or R⁶ are hydroxyl and the other of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy substituted with halo (e.g., fluoro, chloro or bromo) which may be substituted. In particular embodiments five of R¹, R², R³, R⁴, R⁵, and/or R⁶ are hydroxyl and the other of R¹, R², R³, R⁴, R⁵, and/or R⁶ is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexanehexol is a compound of the formula I, II, III or IV wherein five of R¹, R², R³, R⁴, R⁵, and/or R⁶ are hydroxyl and the other of R¹, R², R³, R⁴, R⁵, and/or R⁶ is a haloalkoxyalkyl, in particular fluoromethoxymethyl, chloromethoxyethyl, trifluoromethoxymethyl, difluoromethoxyl, ethyl, or trifluoroethoxymethyl.

In embodiments of the invention, the cyclohexanehexol is a compound of the formula I, II, III or IV wherein R¹, R², R³, R⁴, and R⁵ are hydroxyl and R⁶ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl.

In embodiments of the invention, the cyclohexanehexol is a compound of the formula I, II, III or IV wherein R¹, R², R³, R⁴, and R⁶ are hydroxyl and R⁵ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl, more particularly C₁-C₃ alkyl.

In embodiments of the invention, the cyclohexanehexol is a compound of the formula I, II, III or IV wherein R¹, R², R³, R⁵, and R⁶ are hydroxyl and R⁴ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl, more particularly C₁-C₃ alkyl.

In embodiments of the invention, the cyclohexanehexol is a compound of the formula I, II, III or IV wherein R¹, R², R⁴, and R⁶ are hydroxyl and R³ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl, more particularly C₁-C₃ alkyl.

In embodiments of the invention, the cyclohexanehexol is a compound of the formula I, II, III or IV wherein R¹, R³, R⁴, R⁵, and R⁶ are hydroxyl and R² is substituted alkoxy, in particular alkoxy having about 1-6 carbon atoms, more particularly methoxy, ethoxy, propoxy, butoxy, isopropoxy and tert-butoxy substituted with alkyl, in particular lower alkyl, more particularly C₁-C₃ alkyl.

In embodiments of the invention, the cyclohexanehexol is a compound of the formula I, II, III or IV wherein R², R³, R⁴, R⁵, and R⁶ are hydroxyl and R¹ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl, more particularly C₁-C₃ alkyl.

In embodiments of the invention, the cyclohexanehexol is a compound of the formula I, II, III or IV wherein R¹, R², R³, R⁴, and R⁵ are hydroxyl and R⁶ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R³, R⁴, and R⁵ are hydroxyl and R⁶ is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexanehexol is a compound of the formula I, II, III or IV wherein R¹, R², R³, R⁴, and R⁶ are hydroxyl and R⁵ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R³, R⁴, and R⁶ are hydroxyl and R⁵ is is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexanehexol is a compound of the formula I, II, III or IV wherein R¹, R², R³, R⁵, and R⁶ are hydroxyl and R⁴ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R³, R⁴, and R⁶ are hydroxyl and R⁵ is is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexanehexol is a compound of the formula I, II, III or IV wherein R¹, R², R⁴, R⁵, and R⁶ are hydroxyl and R³ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R⁴, R⁵, and R⁶ are hydroxyl and R³ is is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexanehexol is a compound of the formula I, II, III or IV wherein R¹, R³, R⁴, R⁵, and R⁶ are hydroxyl and R² is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R³, R⁴, R⁵, and R⁶ are hydroxyl and R² is is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexanehexol is a compound of the formula I, II, III or IV wherein R², R³, R⁴, R⁵, and R⁶ are hydroxyl and R¹ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R², R³, R⁴, R⁵, and R⁶ are hydroxyl and R¹ is is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexanehexol is a compound of the formula I, II, III or IV wherein one, two, three, four or five of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is a carboxylic ester. In aspects of the invention at least one of R¹, R², R³, R⁴, R⁵, and/or R⁶ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexanehexol is a compound of the formula I, II, III or IV wherein two of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is a carboxylic ester.

In embodiments of the invention, the cyclohexanehexol is a compound of the formula I, II, III or IV wherein three of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is a carboxylic ester.

In embodiments of the invention, the cyclohexanehexol is a compound of the formula I, II, III or IV wherein four of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is a carboxylic ester.

In embodiments of the invention, the cyclohexanehexol is a compound of the formula I, II, III or IV wherein five of R¹, R², R³, R⁴, R⁵, or R⁶ are hydroxyl and the other of R¹, R², R³, R⁴, R⁵, or R⁶ is a carboxylic ester.

In embodiments of the invention, the cyclohexanehexol is a compound of the formula I, II, III or IV wherein at least one of R¹, R², R³, R⁴, R⁵, and/or R⁶ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexanehexol is a compound of the formula I, II, III or IV wherein R¹, R², R³, R⁴, and R⁵ are hydroxyl and R⁶ is a carboxylic ester. In aspects of the invention, R⁶ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexanehexol is a compound of the formula I, II, III or IV wherein R¹, R², R³, R⁴, and R⁶ are hydroxyl and R⁵ is a carboxylic ester. In aspects of the invention, R⁵ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexanehexol is a compound of the formula I, II, III or IV wherein R¹, R², R³, R⁵, and R⁶ are hydroxyl and R⁴ is a carboxylic ester. In aspects of the invention, R⁴ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexanehexol is a compound of the formula I, II, III or IV wherein R¹, R², R⁴, R⁵, and R⁶ are hydroxyl and R³ is a carboxylic ester. In aspects of the invention, R³ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexanehexol is a compound of the formula I, II, III or IV wherein R¹, R³, R⁴, R⁵, and R⁶ are hydroxyl and R² is a carboxylic ester. In aspects of the invention, R² is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexanehexol is a compound of the formula I, II, III or IV wherein R¹, R³, R⁴, R⁵, and R⁶ are hydroxyl and R² is a carboxylic ester. In aspects of the invention, R² is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic. In particular embodiments, R¹⁴ is selected to provide an amino acid derivative or an ester derivative.

In preferred embodiments of the invention R¹⁴ is one of the following:

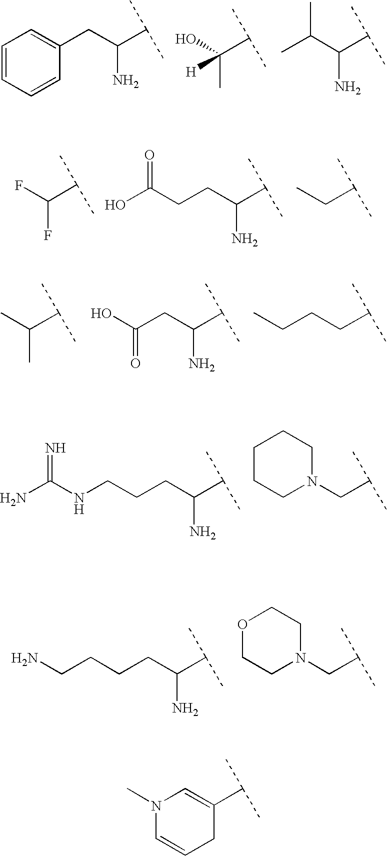

In embodiments of the invention, the cyclohexanehexol is a compound of the formula I, II, III or IV wherein one, two or three of R¹, R², R³, R⁴, R⁵, and/or R⁶ is each indenpendently

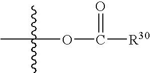

where R³⁰ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, and the other of R¹, R², R³, R⁴, R⁵, and/or R⁶ is hydroxyl.

In embodiments of the invention, the cyclohexanehexol is a compound of the formula I, II, III or IV wherein at least one, two, three or four of R¹, R³, R⁴, R⁵, and/or R⁶ are hydroxyl and the other of R¹, R³, R⁴, R⁵, and/or R⁶ are alkyl, halo, alkoxy, sulfonyl, sulfinyl, thiol, thioalkyl, thioalkoxy, carboxyl, in particular C₁-C₆ alkyl, C₁-C₆ alkoxy, or halo.

In embodiments of the invention, the cyclohexanehexol is a compound of the formula I, II, III or IV wherein R¹, R², R³, R⁴, R⁵, and/or R⁶ is each independently —CH₃, —OCH₃, F, N₃, NH₂, SH, NO₂, CF₃, OCF₃, SeH, Cl, Br, I or CN with the proviso that four or five of R¹, R², R³, R⁴, R⁵, and/or R⁶ are hydroxyl.

In embodiments of the invention, the cyclohexanehexol is a compound of the formula I, II, III or IV wherein five of R¹, R², R³, R⁴, R⁵, and/or R⁶ are hydroxyl and one of R¹, R², R³, R⁴, R⁵, or R⁶, and more particularly R² or R³, is selected from the group consisting of —CH₃, —OCH₃, CF₃, F, SH, SeH, Cl, Br, I and CN.

In embodiments of the invention, the cyclohexanehexol is a compound of the formula I, II, III or IV wherein four of R¹, R², R³, R⁴, R⁵, and/or R⁶ are hydroxyl and two of R¹, R², R³, R⁴, R⁵, and/or R⁶ are selected from the group consisting of —CH₃, —OCH₃, CF₃, F, —NO₂, SH, SeH, Cl, Br, I and CN.

In embodiments of the invention, the cyclohexanehexol is a compound of the formula III or IV, wherein four of R¹, R², R³, R⁴, R⁵, or R⁶ are hydroxyl; one of R¹, R², R³, R⁴, R⁵, or R⁶ is each independently selected from the group CH₃, OCH₃, NO₂, CF₃, OCF₃, F, SH, Cl, Br, I and CN.

In embodiments of the invention, the cyclohexanehexol is a compound of the formula R⁵ or R⁶ are hydroxyl; and one of R¹, R², R³, R⁴, R⁵, or R⁶ is selected from CH₃, OCH₃, NO₂, CF₃, OCF₃, SH, F, Cl, Br, I and CN.

In embodiments of the invention, the cyclohexanehexol is a compound of the formula I, II, III or IV wherein four of R¹, R², R³, R⁴, R⁵, and/or R⁶ are hydroxyl and the other two of R¹, R², R³, R⁴, R⁵, and/or R⁶ are lower alkyl, especially methyl, ethyl, butyl, or propyl, preferably methyl.

In embodiments of the invention, the cyclohexanehexol is a compound of the formula I, II, III or IV wherein four of R¹, R², R³, R⁴, R⁵, and/or R⁶ are hydroxyl and the other two of R¹, R², R³, R⁴, R⁵, and/or R⁶ are lower cycloalkyl, especially cyclopropyl, cyclobutyl, and cyclopentyl.

In embodiments of the invention, the cyclohexanehexol is a compound of the formula I, II, III or IV wherein two, three, four or five of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is halo, in particular fluoro, chloro or bromo, more particularly chloro.

In embodiments of the invention, the cyclohexanehexol is a compound of the formula I, II, III or IV wherein two of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is halo, in particular fluoro, chloro or bromo, more particularly chloro.

In embodiments of the invention, the cyclohexanehexol is a compound of the formula I, II, III or IV wherein three of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is halo, in particular fluoro, chloro or bromo, more particularly chloro.

In embodiments of the invention, the cyclohexanehexol is a compound of the formula I, II, III or IV wherein four of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, or R⁶ is halo, in particular fluoro, chloro or bromo, more particularly chloro.

In embodiments of the invention, the cyclohexanehexol is a compound of the formula III or IV, wherein two, three, four or five of R¹, R², R³, R⁴, R⁵, or R⁶ are hydroxyl; at least one of R¹, R², R³, R⁴, R⁵, or R⁶ is halo; and the remainder of R¹, R², R³, R⁴, R⁵, or R⁶, if any, are independently C₁-C₆alkyl, C₂-C₆ alkenyl, C₂-C₆alkynyl, C₁C₆alkoxy, C₂-C₆alkenyloxy, C₃-C₁₀cycloalkyl, C₁-C₆acyl, C₁-C₆ acyloxy, —NH₂, —NHR⁷, —NR⁷R⁸—, ═NR⁷, —S(O)₂R⁷, —SH, nitro, cyano, halo, haloalkyl, haloalkoxy, hydroxyalkyl, —CO₂R⁷, oxo, —PO₃H—NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀ heteroaryl and C₃-C_{10B}heterocyclic.

In still another aspect, the cyclohexanehexol is a compound of formula III or IV, wherein four of R¹, R², R³, R⁴, R⁵, or R⁶ are hydroxyl; one of R¹, R², R³, R⁴, R⁵, or R⁶ is halo; and one of R¹, R², R³, R⁴, R⁵, or R⁶ is selected from C₁-C₆alkyl, C₂-C₆alkenyl, C₂-C₆alkynyl, C₁C₆alkoxy, C₂-C₆alkenyloxy, C₃-C₁₀cycloalkyl, C₁-C₆ acyl, C₁-C₆ acyloxy, hydroxyl, —NH₂, —NHR⁷, —NR⁷R⁸—, ═NR⁷, —S(O)₂R⁷, —SH, nitro, cyano, halo, haloalkyl, haloalkoxy, hydroxyalkyl, —Si(RP^{7P})B_{3B}, —CO₂R⁷, oxo, —PO₃H—NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀ heteroaryl and C₃-C₁₀heterocyclic, and at least one of R¹, R², R³, R⁴, R⁵, or R⁶ is halo.

In embodiments of the invention, the cyclohexanehexol is a compound of the formula I, II, III or IV wherein five of R¹, R², R³, R⁴, R⁵, and/or R⁶ are hydroxyl and the other of R¹, R², R³, R⁴, R⁵, and/or R⁶ is halo, in particular fluoro, chloro or bromo, more particularly chloro.

In embodiments of the invention, the cyclohexanehexol is a compound of the formula I, II, III or IV wherein R¹, R², R³, R⁴, and R⁵ are hydroxyl and R⁶ is halo, in particular fluorine, chlorine or bromine, more particularly chloro. In a particular embodiment of the invention, R¹, R², R³, R⁴, and R⁵ are hydroxyl and R⁶ is chloro.

In embodiments of the invention, the cyclohexanehexol is a compound of the formula I, II, III or IV wherein R¹, R², R³, R⁴, and R⁶ are hydroxyl and R⁵ is halo, in particular fluoro, chloro or bromo, more particularly chloro. In a particular embodiment of the invention, R¹, R², R³, R⁴, and R⁶ are hydroxyl and R⁵ is chloro.

In embodiments of the invention, the cyclohexanehexol is a compound of the formula I, II, III or IV wherein R¹, R², R³, R⁵, and R⁶ are hydroxyl and R⁴ is halo, in particular fluoro, chloro or bromo, more particularly chloro. In a particular embodiment of the invention, R¹, R², R³, R⁵, and R⁶ are hydroxyl and R⁴ is chloro.

In embodiments of the invention, the cyclohexanehexol is a compound of the formula I, II, III or IV wherein R¹, R², R⁴, R⁵, and R⁶ are hydroxyl and R³ is halo, in particular fluoro, chloro or bromo, more particularly chloro. In a particular embodiment of the invention, R¹, R², R⁴, R⁵, and R⁶ are hydroxyl and R³ is chloro.

In embodiments of the invention, the cyclohexanehexol is a compound of the formula I, II, III or IV wherein R¹, R³, R⁴, R⁵, and R⁶ are hydroxyl and R² is halo, in particular fluoro, chloro or bromo, more particularly chloro. In a particular embodiment of the invention, R¹, R³, R⁴, R⁵, and R⁶ are hydroxyl and R² is chloro.

In embodiments of the invention, the cyclohexanehexol is a compound of the formula I, II, III or IV wherein R², R³, R⁴, R⁵, and R⁶ are hydroxyl and R¹ is halo, in particular fluoro, chloro or bromo, more particularly chloro. In a particular embodiment of the invention, R², R³, R⁴, R⁵, and R⁶ are hydroxyl and R¹ is chloro.

In aspects of the invention, the cyclohexanehexol is a scyllo-inositol compound, in particular a pure or substantially pure scyllo-inositol compound.

A “scyllo-inositol compound” includes compounds having the structure of the formula Va or Vb:

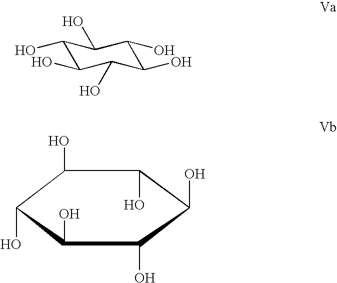

A scyllo-inositol compound includes a compound of the formula Va or Vb wherein one to six, one to five, one, two, three or four, preferably one, two or three, more preferably one or two hydroxyl groups are replaced by substituents, in particular univalent substituents, with retention of configuration. In aspects of the invention, a scyllo-inositol compound comprises a compound of the formula Va or Vb wherein one, two, three, four, five or six, preferably one or two, most preferably one, hydroxyl groups are replaced by univalent substituents, with retention of configuration. Suitable substituents include without limitation hydrogen; alkyl; substituted alkyl; acyl; alkenyl; substituted alkenyl; alkynyl; substituted alkynyl; cycloalkyl; substituted cycloalkyl; alkoxy; substituted alkoxy; aryl; aralkyl; substituted aryl; halogen; thiol; —NHR⁴¹ wherein R⁴¹ is hydrogen, acyl, alkyl or —R⁴²R⁴³ wherein R⁴² and R⁴³ are the same or different and represent acyl or alkyl; —PO₃H₂; —SR⁴⁴ wherein R⁴⁴ is hydrogen, alkyl, or —O₃H; or —OR⁴⁵ wherein R⁴⁵ is hydrogen, alkyl, or —SO₃H.

Particular aspects of the invention utilize scyllo-inositol compounds of the formula Va or Vb wherein one or more of the hydroxyl groups is replaced with alkyl, in particular C₁-C₄ alkyl, more particularly methyl; acyl; chloro or fluoro; alkenyl; —NHR⁴¹ wherein R⁴¹ is hydrogen, acyl, alkyl or —R⁴²R⁴³ wherein R⁴² and R⁴³ are the same or different and represent acyl or alkyl; —SR⁴⁴ wherein R⁴⁴ is hydrogen, alkyl, or —O₃H; and —OR⁴⁵ wherein R⁴⁵ is hydrogen, alkyl, or —SO₃H, more particularly —SR⁴⁴ wherein R⁴⁴ is hydrogen, alkyl, or —O₃H or —OR⁴⁵ wherein R⁴⁵ is —SO₃H.

Particular aspects of the invention utilize scyllo-inositol compounds of the formula Va or Vb wherein one or more of the hydroxyl groups is replaced with alkyl; substituted alkyl; acyl; alkenyl; substitututed alkenyl; —NHR⁴¹ wherein R⁴¹ is hydrogen, acyl, alkyl, or —R⁴²R⁴³ wherein R⁴² and R⁴³ are the same or different and represent acyl or alkyl; —SR⁴⁴ wherein R⁴⁴ is hydrogen, alkyl, or —O₃H; or —OR⁴⁵ wherein R⁴⁵ is hydrogen, alkyl or —SO₃H.

Particular aspects of the invention utilize scyllo-inositol compounds of the formula Va or Vb wherein one or more of the hydroxyl groups is replaced with alkyl; substituted alkyl; acyl; alkenyl; substituted alkenyl; alkynyl; substituted alkynyl; alkoxy; substituted alkoxy; halogen; thiol; —NHR⁴¹ wherein R⁴¹ is hydrogen, acyl, alkyl or —R42R⁴³ wherein R⁴² and R⁴³ are the same or different and represent acyl or alkyl; —PO₃H₂; —SR⁴⁴ wherein R⁴⁴ is hydrogen, alkyl, or —O₃H; —OR⁴⁵ wherein R⁴⁵ is hydrogen, alkyl, or —OR⁴⁵ wherein R⁴⁵ is —SO₃H.

Particular aspects of the invention utilize scyllo-inositol compounds of the formula Va or Vb wherein one or more of the hydroxyl groups is replaced with alkyl; substituted alkyl; acyl; alkenyl; substituted alkenyl; alkynyl; substituted alkynyl; alkoxy; substituted alkoxy; halogen; or thiol.

Particular aspects of the invention utilize scyllo-inositol compounds of the formula Va or Vb wherein one of the hydroxyl groups is replaced with alkyl, in particular C₁-C₄ alkyl, more particularly methyl.

Particular aspects of the invention utilize scyllo-inositol compounds of the formula Va or Vb wherein one of the hydroxyl groups is replaced with halogen, in particular chloro or fluoro, more particularly fluoro.

Particular aspects of the invention utilize scyllo-inositol compounds of the formula Va or Vb wherein one of the hydroxyl groups is replaced with thiol.

In embodiments of the invention, the scyllo-inositol compound designated AZD-103/ELND005 (Elan Corporation) is used in the formulations, dosage forms, methods and uses disclosed herein.

In embodiments of the invention, the cyclohexanehexol is methyl-scyllo-inositol

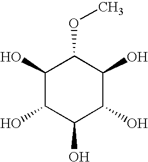

In embodiments of the invention, the cyclohexanehexol is 1-chloro-1-deoxy-scyllo-inositol.

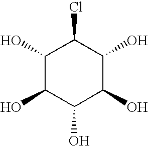

In aspects of the invention, the cyclohexanehexol is an epi-inositol compound, in particular a pure or substantially pure epi-inositol compound.

An “epi-inositol compound” includes compounds having the base structure of formula VI:

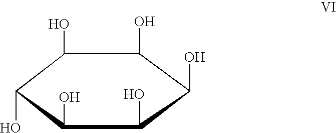

An epi-inositol compound includes a compound of the formula VI wherein one to six, one to five, one, two, three or four, preferably one, two or three, more preferably one or two hydroxyl groups are replaced by substituents, in particular univalent substituents, with retention of configuration. In aspects of the invention, an epi-inositol compound comprises a compound of the formula VI wherein one, two, three, four, five or six, preferably one or two, most preferably one, hydroxyl groups are replaced by univalent substituents, with retention of configuration. Suitable substituents include without limitation hydrogen; alkyl; substituted alkyl; acyl; alkenyl; substituted alkenyl; alkynyl; substituted alkynyl; cycloalkyl; substituted cycloalkyl; alkoxy; substituted alkoxy; aryl; aralkyl; substituted aryl; halogen; thiol; —NHR⁴¹ wherein R⁴¹ is hydrogen, acyl, alkyl or —R⁴²R⁴³ wherein R⁴² and R⁴³ are the same or different and represent acyl or alkyl; —PO₃H₂; —SR⁴⁴ wherein R⁴⁴ is hydrogen, alkyl, or —O₃H; or —OR⁴⁵ wherein R⁴⁵ is hydrogen, alkyl, or —SO₃H.

Particular aspects of the invention utilize epi-inositol compounds of the formula Va or Vb wherein one or more of the hydroxyl groups is replaced with alkyl, in particular C₁-C₄ alkyl, more particularly methyl; acyl; chloro or fluoro; alkenyl; —NHR⁴¹ wherein R⁴¹ is hydrogen, acyl, alkyl or —R⁴²R⁴³ wherein R⁴² and R⁴³ are the same or different and represent acyl or alkyl; —SR⁴⁴ wherein R⁴⁴ is hydrogen, alkyl, or —O₃H; and —OR⁴⁵ wherein R⁴⁵ is hydrogen, alkyl, or —SO₃H, more particularly —SR⁴⁴ wherein R⁴⁴ is hydrogen, alkyl, or —O₃H or —OR⁴⁵ wherein R⁴⁵ is —SO₃H.

Particular aspects of the invention utilize epi-inositol compounds of the formula Va or Vb wherein one or more of the hydroxyl groups is replaced with alkyl; substituted alkyl; acyl; alkenyl; substituted alkenyl; —NHR⁴¹ wherein R⁴¹ is hydrogen, acyl, alkyl, or —R⁴²R⁴³ wherein R⁴² and R⁴³ are the same or different and represent acyl or alkyl; —SR⁴⁴ wherein R⁴⁴ is hydrogen, alkyl, or —O₃H; or —OR⁴⁵ wherein R⁴⁵ is hydrogen, alkyl or —SO₃H.

Particular aspects of the invention utilize epi-inositol compounds of the formula Va or Vb wherein one or more of the hydroxyl groups is replaced with alkyl; substituted alkyl; acyl; alkenyl; substituted alkenyl; alkynyl; substituted alkynyl; alkoxy; substituted alkoxy; halogen; thiol; —NHR⁴¹ wherein R⁴¹ is hydrogen, acyl, alkyl or —R⁴²R⁴³ wherein R⁴² and R⁴³ are the same or different and represent acyl or alkyl; —PO₃H₂; —SR⁴⁴ wherein R⁴⁴ is hydrogen, alkyl, or —O₃H; —OR⁴⁵ wherein R⁴⁵ is hydrogen, alkyl, or —OR⁴⁵ wherein R⁴⁵ is —SO₃H.

Particular aspects of the invention utilize epi-inositol compounds of the formula Va or Vb wherein one or more of the hydroxyl groups is replaced with alkyl; substituted alkyl; acyl; alkenyl; substituted alkenyl; alkynyl; substituted alkynyl; alkoxy; substituted alkoxy; halogen; or thiol.

Particular aspects of the invention utilize epi-inositol compounds of the formula Va or Vb wherein one of the hydroxyl groups is replaced with alkyl, in particular C₁-C₄ alkyl, more particularly methyl.

Particular aspects of the invention utilize epi-inositol compounds of the formula Va or Vb wherein one of the hydroxyl groups is replaced with halogen, in particular chloro or fluoro, more particularly fluoro.

Particular aspects of the invention utilize epi-inositol compounds of the formula Va or Vb wherein one of the hydroxyl groups is replaced with thiol.

In aspects of the invention, the cyclohexanehexol is epi-inositol, in particular a pure or substantially pure epi-inositol.

Cyclohexanehexols utilized in the invention may be prepared using reactions and methods generally known to the person of ordinary skill in the art, having regard to that knowledge and the disclosure of this application. The reactions are performed in a solvent appropriate to the reagents and materials used and suitable for the reactions being effected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired compound of the invention. It will also be recognized that another major consideration in the development of a synthetic route is the selection of the protecting group used for protection of the reactive functional groups present in the compounds described in this invention. An authoritative account describing the many alternatives to the skilled artisan is Greene and Wuts (Protective Groups In Organic Synthesis, Wiley and Sons, 1991).

The starting materials and reagents used in preparing cyclohexanehexols are either available from commercial suppliers such as the Aldrich Chemical Company (Milwaukee, Wis.), Bachem (Torrance, Calif.), Sigma (St. Louis, Mo.), or Lancaster Synthesis Inc. (Windham, N.H.) or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

The starting materials, intermediates, and cyclohexanehexols may be isolated and purified using conventional techniques, such as precipitation, filtration, distillation, crystallization, chromatography, and the like. The compounds may be characterized using conventional methods, including physical constants and spectroscopic methods, in particular HPLC.

Cyclohexanehexols which are basic in nature can form a wide variety of different salts with various inorganic and organic acids. In practice it is desirable to first isolate a cyclohexanehexol from the reaction mixture as a pharmaceutically unacceptable salt and then convert the latter to the free base compound by treatment with an alkaline reagent and subsequently convert the free base to a pharmaceutically acceptable acid addition salt. The acid addition salts of the base compounds are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

Cyclohexanehexols which are acidic in nature are capable of forming base salts with various pharmacologically acceptable cations. These salts may be prepared by conventional techniques by treating the corresponding acidic compounds with an aqueous solution containing the desired pharmacologically acceptable cations and then evaporating the resulting solution to dryness, preferably under reduced pressure. Alternatively, they may be prepared by mixing lower alkanolic solutions of the acidic compounds and the desired alkali metal alkoxide together and then evaporating the resulting solution to dryness in the same manner as before. In either case, stoichiometric quantities of reagents are typically employed to ensure completeness of reaction and maximum product yields.

Scyllo-inositol compounds may be prepared using conventional processes or they may be obtained from commercial sources. For example, scyllo-inositol compounds can be prepared using chemical and/or microbial processes. In aspects of the invention, a scyllo-inositol is produced using process steps described by M. Sarmah and Shashidhar, M., Carbohydrate Research, 2003, 338, 999-100, Husson, C., et al, Carbohyrate Research 307 (1998) 163-165; Anderson R. and E. S. Wallis, J. American Chemical Society (US), 1948, 70:2931-2935; Weissbach, A., J Org Chem (US), 1958, 23:329-330; Chung, S. K. et al., Bioorg Med Chem. 1999, 7(11):2577-89; or Kiely D. E., and Fletcher, H. G., J. American Chemical Society (US) 1968, 90:3289-3290; described in JP09-140388, DE 3,405,663 (Merck Patent GMBH), JP04-126075, JP05-192163, or WO06109479, or described in WO0503577, US20060240534, EP1674578, JP9140388, JP09140388, JP02-184912, JP03-102492 (Hokko Chemical Industries). In particular aspects of the compositions and methods of the invention, a scyllo-inositol is prepared using the chemical process steps described in Husson, C., et al, Carbohydrate Research 307 (1998) 163-165. In other aspects of the compositions and methods of the invention, a scyllo-inositol is prepared using microbial process steps similar to those described in WO05035774 (EP1674578 and US20060240534) JP2003102492, or JP09140388 (Hokko Chemical Industries). Derivatives may be produced by introducing substituents into a scyllo-cyclohexanehexol using methods well known to a person of ordinary skill in the art.

In aspects of the invention, an epi-inositol can be prepared using chemical and/or microbial processes. For example, an epi-inositol may be prepared by the process described by V. Pistard (Tetrahedron Letters 41, 3253, 2000), Magasanik B., and Chargaff E. (J Biol Chem, 1948, 174:173188), U.S. Pat. No. 7,157,268, or in PCT Published Application No. WO0075355. Derivatives may be produced by introducing substituents into an epi-inositol using methods well known to a person of ordinary skill in the art.

A cyclohexanehexol may additionally comprise a carrier, including without limitation one or more of a polymer, carbohydrate, peptide or derivative thereof. A carrier may be substituted with substituents described herein including without limitation one or more alkyl, amino, nitro, halogen, thiol, thioalkyl, sulfate, sulfonyl, sulfenyl, sulfinyl, sulfoxide, hydroxyl groups. A carrier can be directly or indirectly covalently attached to a compound of the invention. In aspects of the invention the carrier is an amino acid including alanine, glycine, proline, methionine, serine, threonine, or asparagine. In other aspects the carrier is a peptide including alanyl-alanyl, prolyl-methionyl, or glycyl-glycyl.

A carrier also includes a molecule that targets a cyclohexanehexol to a particular tissue or organ. In particular, a carrier may facilitate or enhance transport of a compound of the invention to the brain by either active or passive transport.

A “polymer” as used herein refers to molecules comprising two or more monomer subunits that may be identical repeating subunits or different repeating subunits. A monomer generally comprises a simple structure, low-molecular weight molecule containing carbon. Polymers can be optionally substituted. Examples of polymers which can be used in the present invention are vinyl, acryl, styrene, carbohydrate derived polymers, polyethylene glycol (PEG), polyoxyethylene, polymethylene glycol, poly-trimethylene glycols, polyvinylpyrrolidone, polyoxyethylene-polyoxypropylene block polymers, and copolymers, salts, and derivatives thereof. In particular aspects of the invention, the polymer is poly(2-acrylamido-2-methyl-1-propanesulfonic acid); poly(2-acrylamido-2-methyl,-1-propanesulfonic acid-coacrylonitrile, poly(2-acrylamido-2-methyl-1-propanesulfonic acid-co-styrene), poly(vinylsulfonic acid); poly(sodium 4-styrenesulfonic acid); and sulfates and sulfonates derived therefrom; poly(acrylic acid), poly(methylacrylate), poly(methyl methacrylate), and poly(vinyl alcohol).

A “carbohydrate” as used herein refers to a polyhydroxyaldehyde, or polyhydroxyketone and derivatives thereof. The simplest carbohydrates are monosaccharides, which are small straight-chain aldehydes and ketones with many hydroxyl groups added, usually one on each carbon except the functional group. Examples of monosaccharides include erythrose, arabinose, allose, altrose, glucose, mannose, threose, xylose, gulose, idose, galactose, talose, aldohexose, fructose, ketohexose, ribose, and aldopentose. Other carbohydrates are composed of monosaccharide units, including disaccharides, oligosaccharides, or polysaccharides, depending on the number of monosaccharide units. Disaccharides are composed of two monosaccharide units joined by a covalent glycosidic bond. Examples of disaccharides are sucrose, lactose, and maltose. Oligosaccharides and polysaccharides, are composed of longer chains of monosaccharide units bound together by glycosidic bonds. Oligosaccharides generally contain between 3 and 9 monosaccharide units and polysaccharides contain greater than 10 monosaccharide units. A carbohydrate group may be substituted at one two, three or four positions, other than the position of linkage to a compound of the formula I, II, III or IV. For example, a carbohydrate may be substituted with one or more alkyl, amino, nitro, halo, thiol, carboxyl, or hydroxyl groups, which are optionally substituted. Illustrative substituted carbohydrates are glucosamine or galactosamine.

In aspects of the invention, the carbohydrate is a sugar, in particular a hexose or pentose and may be an aldose or a ketose. A sugar may be a member of the D or L series and can include amino sugars, deoxy sugars, and their uronic acid derivatives. In embodiments of the invention where the carbohydrate is a hexose, the hexose is selected from the group consisting of glucose, galactose, or mannose, or substituted hexose sugar residues such as an amino sugar residue such as hexosamine, galactosamine, glucosamine, in particular D-glucosamine (2-amino-2-doexy-D-glucose) or D-galactosamine (2-amino-2-deoxy-D-galactose). Suitable pentose sugars include arabinose, fucose, and ribose.

The term “carbohydrate” also includes glycoproteins such as lectins (e.g. concanavalin A, wheat germ agglutinin, peanutagglutinin, seromucoid, and orosomucoid) and glycolipids such as cerebroside and ganglioside.

A “peptide” for use as a carrier in the practice of the present invention includes one, two, three, four, or five or more amino acids covalently linked through a peptide bond. A peptide can comprise one or more naturally occurring amino acids, and analogs, derivatives, and congeners thereof. A peptide can be modified to increase its stability, bioavailability, solubility, etc. “Peptide analogue” and “peptide derivative” as used herein include molecules which mimic the chemical structure of a peptide and retain the functional properties of the peptide. In aspects of the invention, the carrier is an amino acid such as alanine, glycine, proline, methionine, serine, threonine, histidine, or asparagine. In other aspects the carrier is a peptide such as alanyl-alanyl, prolyl-methionyl, or glycyl-glycyl. In still other aspects, the carrier is a polypeptide such as albumin, antitrypsin, macroglobulin, haptoglobin, caeruloplasm, transferrin, α- or β-lipoprotein, β- or γ-globulin or fibrinogen.

Approaches to designing peptide analogues, derivatives and mimetics are known in the art. For example, see Farmer, P. S. in Drug Design (E. J. Ariens, ed.) Academic Press, New York, 1980, vol. 10, pp. 119-143; Ball. J. B. and Alewood, P. F. (1990) J Mol. Recognition 3:55; Morgan, B. A. and Gainor, J. A. (1989) Ann. Rep. Med. Chem. 24:243; and Freidinger, R. M. (1989) Trends Pharmacol. Sci. 10:270. See also Sawyer, T. K. (1995) “Peptidomimetic Design and Chemical Approaches to Peptide Metabolism” in Taylor, M. D. and Amidon, G. L. (eds.) Peptide-Based Drug Design: Controlling Transport and Metabolism, Chapter 17; Smith, A. B. 3rd, et al. (1995) J. Am. Chem. Soc. 117:11113-11123; Smith, A. B. 3rd, et al. (1994) J. Am. Chem. Soc. 116:9947-9962; and Hirschman, R., et al. (1993) J. Am. Chem. Soc. 115:12550-12568.

Examples of peptide analogues, derivatives and peptidomimetics include peptides substituted with one or more benzodiazepine molecules (see e.g., James, G. L. et al. (1993) Science 260:1937-1942), peptides with methylated amide linkages and “retro-inverso” peptides (see U.S. Pat. No. 4,522,752 by Sisto).

Examples of peptide derivatives include peptides in which an amino acid side chain, the peptide backbone, or the amino- or carboxy-terminus has been derivatized (e.g., peptidic compounds with methylated amide linkages).

The term mimetic, and in particular, peptidomimetic, is intended to include isosteres. The term “isostere” refers to a chemical structure that can be substituted for a second chemical structure because the steric conformation of the first structure fits a binding site specific for the second structure. The term specifically includes peptide back-bone modifications (i.e., amide bond mimetics) well known to those skilled in the art. Such modifications include modifications of the amide nitrogen, the alpha-carbon, amide carbonyl, complete replacement of the amide bond, extensions, deletions or backbone crosslinks. Other examples of isosteres include peptides substituted with one or more benzodiazepine molecules (see e.g., James, G. L. et al. (1993) Science 260:1937-1942)

Other possible modifications include an N-alkyl (or aryl) substitution ([CONR]), backbone crosslinking to construct lactams and other cyclic structures, substitution of all D-amino acids for all L-amino acids within the compound (“inverso” compounds) or retro-inverso amino acid incorporation ([NHCO]). By “inverso” is meant replacing L-amino acids of a sequence with D-amino acids, and by “retro-inverso” or “enantio-retro” is meant reversing the sequence of the amino acids (“retro”) and replacing the L-amino acids with D-amino acids. For example, if the parent peptide is Thr-Ala-Tyr, the retro modified form is Tyr-Ala-Thr, the inverso form is thr-ala-tyr, and the retro-inverso form is tyr-ala-thr (lower case letters refer to D-amino acids). Compared to the parent peptide, a retro-inverso peptide has a reversed backbone while retaining substantially the original spatial conformation of the side chains, resulting in a retro-inverso isomer with a topology that closely resembles the parent peptide. See Goodman et al. “Perspectives in Peptide Chemistry” pp. 283-294 (1981). See also U.S. Pat. No. 4,522,752 by Sisto for further description of “retro-inverso” peptides.

A peptide can be attached to a cyclohexanehexol through a functional group on the side chain of certain amino acids (e.g. serine) or other suitable functional groups. In embodiments of the invention the carrier may comprise four or more amino acids with groups attached to three or more of the amino acids through functional groups on side chains. In another embodiment, the carrier is one amino acid, in particular a sulfonate derivative of an amino acid, for example cysteic acid.

An “NSAID” is a non-steroidal anti-inflammatory drug including organic acids that have analgesic, anti-inflammatory, and anti-pyretic effects. Classes of NSAIDs include without limitation amino aryl carboxylic acid derivatives (for example, flufenamic acid, mefenamic acid and meclofenamic acid); aryl acetic acid derivatives (for example, diclofenac, aceclofenac, acemetacin, bromfenac, etodolac, nabumetone, tolmetin, indomethacin and sulindac); aryl propionic acid derivatives (for example, fenoprofen, ibuprofen, fenbufen, flurbiprofen, ketoprofen, ketorolac, loxoprofen, naproxen, oxaprozin, tiaprofenic acid, suprofen and carprofen); salicylates (for example, aspirin, amoxiprin, benorilate, choline magnesium salicylate, diflunisal, faislamine, methyl salicylate, magnesium salicylate, and salicyl salicylate); pyrazolidines (for example, phenylbutazone, azapropazone, metamizole, oxyphenbutazone, and sulfinprazone); and oxicams (for example, piroxicam, lomoxicam, meloxicam, and tenoxicam).

Examples of NSAIDs include ibuprofen (Motrin, Advil, Vicoprofen, Apo-ibuprofen), flurbuprofen (Ansaid, Froben), naproxen (Naprosyn, Naprosyn E, Naprosyn SR, Apo-Naproxen, Naxen, Naprelan, Anaprox, Novo-Naprox), sulindac (Clinoril, Apo-Sulin, Novo-Sudac), diclofenac and diclofenac sodium (Voltaren, Volaren SR, Solaraze, Arthrotec, Apo-Diclo, Apo-Diclo SR, Pennsaid, Novo-Difenac), piroxicam (Feldene, Apo-Piroxicam, Novo-Pirocam), salsalate (Disalcid), ASA (Entrophen), meloxicam (Mobicox), tenoxicam (Mobiflex), ketoprofen (Orudis, Orudis E, Oruvail, Apo-Keto E, Novo-Profen, Rhodis), idflunisal (Dolobid), nabumetone (Relafen), etodolac (Lodine, Ultradol), tiaprofenic acid (Surgam, Surgam SR, AlbertTiafen), fenoprofen (Nalfon), oxaprozin (Daypro), Meclofenamic acid (Meclofen), celecoxib (Celebrex), tolmetin (Tolectin) and indomethacin (Indocin, Apo-Indomethacin, Novo-Methacin).

A “racemic mixture” includes equal or unequal amounts of R- and S-enantiomers of a compound, in particular a NSAID. A racemic mixture generally contains effective amounts of an R- or S-enantiomer to elicit a desired effect specific to the R- or S-enantiomer, respectively. A racemic mixture may contain R- and S-enantiomers having a range of between about 5% to greater than about 95%. In aspects of the invention, the racemic mixture may contain 95% or greater by weight of an R-enantiomer of a NSAID and 5% or lower of the corresponding S-enantiomer of the NSAID, based upon the total amount present in the composition. In specific aspects of the invention, the ratio of R- to S-enantiomers in a racemic mixture is within the range of about: 5:95, 10:90, 20:80, 30:70, 40:60, 50:50, 60:40, 70:30, 80:20, 90:10, or 95:5. In aspects of the invention, the amount of S-NSAID in a racemic mixture is insufficient to elicit an adverse effect in a subject or at most elicits an adverse effect that is tolerable to the subject and is outweighed by the beneficial effect or effects. In specific aspects of the invention, the ratio of R- to S-enantiomers in a racemic mixture is within the range of about: 70:30, 80:20, 90:10, or 95:5.

An “R-NSAID” refers to a NSAID which is a pure or substantially pure R-enantiomer or a component of a racemic mixture. Examples of R-NSAIDs are R-enantiomers of arylpropionic acids, arylacetic acids, and thiazinecarboxamides, etc. In the compositions and methods of the invention, the R-NSAID used is typically adjusted to avoid any adverse effects that may be associated with the S-enantiomer. In aspects of the invention, a composition or method comprises a greater proportion of the R-isomer of a NSAID in relation to the S-isomer.

In aspects, an R-NSAID employed in the present invention can be an R-enantiomer of an arylpropionic acid or an arylacetic acid or a pharmaceutically acceptable salt or ester thereof. In specific aspects of the invention, the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-etodolac and R-benoxaprofen.

The term “R-NSAID” also includes an R-enantiomer of a NSAID functional derivative, in particular a NSAID analog or NSAID derivative. R-enantiomer NSAID derivatives and NSAID analogs include derivatives and analogs that can be metabolized in a subject to form the corresponding acid form of the NSAIDs.

A NSAID can be modified by addition of a group or by substituting functional groups.

For example, a NSAID derivative or analog can be formed by performing substitutions to the aminocarboxylic acid, arylacetic acid, or arylpropionic acid groups of NSAIDs. NSAID analogs and derivatives can also be formed by modifications and additions to indole compounds. For example, alkyl, hydroxyl alkyl, phenyl, benzyl, and/or thienyl groups can be added to indole compounds to form analogs and derivatives. Structural analogs can also be identified using computational methods with commercially available computer modeling programs.

NSAID derivatives include, for example, cycylized derivatives, esters, amides and alcohols. A NSAID can be a cyclized derivative of an arylpropionic acid, arylacetic acid, or thiazinecarboxamide. In aspects of the invention the NSAID is a cyclized derivative of arylpropionic acid, such as R-ketorolac, or an arylacetic acid, such as R-etodolac.

A NSAID can also be an ester of a NSAID, in particular an ester of a R-NSAID, more particularly an ester of an R-enantiomer of an arylpropionic acid NSAID, an ester of an R-enantiomer of a cyclized derivative of an arylpropionic acid NSAID, and an ester of an R-enantiomer of arylacetic acid NSAIDs. In aspects, a NSAID, in particular R-NSAID, is a C₁-C₈ alkyl ester, in particular a C₁-C₆ alkyl ester, including a methyl, ethyl, propyl, isopropyl, butyl, isobutyl, tert-butyl, pentyl, and octyl ester.

A NSAID that can be used in the compositions, conjugates and methods of the present invention includes a nitrosated NSAID, NSAID analog or derivative (a monoxide group (NO) is linked to the NSAID etc.) or a nitrosylated NSAID, NSAID analog or derivative (a nitrogen dioxide group (NO₂) is linked to the NSAID etc.). A nitrosated and/or nitrosylated derivative or analog may release nitric oxide and thereby enhance clearing of Aβ deposits. Nitrosated and/or nitrosylated NSAIDs and NSAID derivatives are described in U.S. Pat. No. 6,593,347.

In an aspect of the invention, flurbiprofen, fenoprofen, or carprofen derivatives are utilized. Such derivatives can be formed by modifications including but not limited to: (i) changing the position of the propionic acid radical on a phenyl ring; (ii) changing the bond between the two phenyl rings; (iii) changing the position or types of substituents on the phenyl ring opposite the propionic acid substituent; and/or (iv) replacing the acetic acid substituent with another substituent such as a carboxylic acid.

In specific aspects, the NSAID derivative or analog is a derivative or analog of R-flurbiprofen. In an embodiment, the R-NSAID is an analog of flurbiprofen such as 2-(2′-fluro-4-biphenylyl)propionic acid and 2-(2,2′-difluroro-4-biphenylyl)propionic acid. In another embodment, the R-NSAID is a derivative of flurbiprofen such as 4′-hydroxyflurbiprofen. In a further embodiment, the R-NSAID is a nitrosated or nitrosylated flurbiprofen or R-flurbiprofen.

The term “R-NSAID” also includes pharmaceutically acceptable salts of an R-enantiomer of a NSAID, NSAID derivative or NSAID analog. Examples of such salts include, but are not limited to, alkali metal salts, alkaline earth salts, and ammonium salts, including without limitation salts of aluminum, calcium, lithium, magnesium, potassium, sodium and zinc. Organic salts may also be used including without limitation lysine, N,N′-dibenzylethylenediamine, chloroprocaine, choline, diethanolamine, ethylenediamine, meglumine (N-methylglucamine), procaine and tris salts.

Methods for making NSAIDs are well known in the art. [See for example, U.S. Pat. Nos. 3,755,427; 4,230,724; 5,556,638; 5,955,504; 5,981,592; and 6,160,018 (flurbiprofen); U.S. Pat. No. 3,641,127 (ketoprofen), U.S. Pat. No. 4,089,969 and Muchowski et al., J. Med Chem. 28(8):1037-1049 (ketorolac).] NSAIDs may also be obtained from commercial sources in the form of racemic mixtures or as optically pure enantiomers. (See Table 1) Optically pure R-NAIDs can be obtained from racemic mixtures using methods known in the art (see for example, U.S. Pat. No. 5,331,000 and U.S. Pat. No. 3,642,127 in respect to isolation of optically pure R-ketoprofen and R-ketorolac, respectively).

A cyclohexanehexol and/or NSAID, in particular R-NSAID, may be pure or substantially pure. In general “pure” means better than 80%, 85%, 90%, 92%, 94%, 95%, 96%, 97%, 98% or 99% pure, and “substantially pure” generally means a compound synthesized such that the compound as made available for consideration into a therapeutic dosage has only those impurities that can not readily nor reasonably be removed by conventional purification processes. “Substantially pure” in relation to a NSAID, in particular R-NSAID, used in a composition, conjugate or method of the invention can mean that the composition, conjugate, or method comprises or uses at least 80%, 85%, 90%, 95%, 96%, 97%, 98%, or 99% by weight of a NSAID based on the total amount of NSAID present.

A “disease(s)” or “disorder(s)” refers to one or more pathological symptoms or syndromes for which either or both a cyclohexanehexol and a NSAID, in particular R-NSAID provide a therapeutic effect. A “disease(s)” includes a condition characterized by abnormal protein folding or aggregation or abnormal amyloid formation, deposition, accumulation or persistence, or amyloid lipid interactions. In some aspects, the term includes conditions characterized by abnormal protein folding or aggregation or amyloid formation, deposition, accumulation or persistence. In particular aspects, the disease is a condition of the central or peripheral nervous system or systemic organ. In more particular aspects the terms includes conditions associated with the formation, deposition, accumulation, or persistence of amyloid or amyloid fibrils, comprising an amyloid protein comprising or selected from the group consisting of Aβ amyloid, AA amyloid, AL amyloid, IAPP amyloid, PrP amyloid, α₂-microglobulin amyloid, transthyretin, prealbumin, and procalcitonin, especially Aβ amyloid and IAPP amyloid. A disease may be a condition where it is desirable to dissociate abnormally aggregated proteins and/or dissolve or disrupt pre-formed or pre-deposited amyloid or amyloid fibril.

In certain aspects of the invention the disease is an amyloidosis. “Amyloidosis” refers to a diverse group of diseases of acquired or hereditary origin and characterized by the accumulation of one of several different types of protein fibrils with similar properties called amyloid. Amyloid can accumulate in a single organ or be dispersed throughout the body. The disease can cause serious problems in the affected areas, which may include the heart, brain, kidneys and digestive tract. The fibrillar composition of amyloid deposits is an identifying characteristic for various amyloid diseases. Intracerebral and cerebrovascular deposits composed primarily of fibrils of beta amyloid peptide (β-AP or Aβ) are characteristic of Alzheimer's disease (both familial and sporadic forms); islet amyloid protein peptide (IAPP; amylin) is characteristic of the fibrils in pancreatic islet cell amyloid deposits associated with type II diabetes; and, β-2-microglobulin is a major component of amyloid deposits which form as a consequence of long term hemodialysis treatment. Prion-associated diseases, such as Creutzfeld-Jacob disease, scrapie, bovine spongiform encephalitis, and the like are characterized by the accumulation of a protease-resistant form of a prion protein (designated as AScr or PrP-27).

Certain diseases or disorders are considered to be primary amyloidoses, in which there is no evidence for preexisting or coexisting disease. Primary amyloidoses are typically characterized by the presence of “amyloid light chain-type” (AL-type) protein fibrils. In secondary amyloidosis there is an underlying chronic inflammatory or infectious disease state (e.g., rheumatoid arthritis, juvenile chronic arthritis, ankylosing spondylitis, psoriasis, Reiter's syndrome, Adult Still's disease, Behcet's Syndrome, Crohn's disease, chronic microbial infections such as osteomyelitis, tuberculosis, and leprosy, malignant neoplasms such as Hodgkin's lymphoma, renal carcinoma, carcinomas of the gut, lung, and urogenital tract, basel cell carcinoma, and hairy cell carcinoma). Secondary amyloidosis is characterized by deposition of AA type fibrils derived from serum amyloid A protein (ApoSSA). Heredofamilial amyloidoses may have associated neuropathic, renal, or cardiovascular deposits of the ATTR transthyretin type, and they include other syndromes having different amyloid components (e.g., familial Mediterranean fever which is characterized by AA fibrils). Other forms of amyloidosis include local forms, characterized by focal, often tumor-like deposits that occur in isolated organs. In addition, amyloidoses are associated with aging, and are commonly characterized by plaque formation in the heart or brain. Amyloidoses includes systemic diseases such as adult-onset disabetes, complications from long-term hemodialysis and consequences of chronic inflammation or plasma cell dyscrasias.

In aspects of the invention, amyloid diseases include without limitation, Alzheimer's disease, Down's syndrome, dementia pugilistica, multiple system atrophy, inclusion body myositosis, hereditary cerebral hemorrhage with amyloidosis of the Dutch type, Nieman-Pick disease type C, cerebral β-amyloid angiopathy, dementia associated with cortical basal degeneration, the amyloidosis of type 2 diabetes, the amyloidosis of chronic inflammation, the amyloidosis of malignancy and Familial Mediterranean Fever, the amyloidosis of multiple myeloma and B-cell dyscrasias, nephropathy with urticaria and deafness (Muckle—Wells syndrome), amyloidosis associated with systemic inflammatory diseases, idiopathic primary amyloidosis associated with myeloma or macroglobulinemia; amyloidosis associated with immunocyte dyscrasia; monoclonal gammopathy; occult dyscrasia; local nodular amyloidosis associated with chronic inflammatory diseases; amyloidosis associated with several immunocyte dyscrasias; familial amyloid polyneuropathy; hereditary cerebral hemorrhage with amyloidosis Alzheimer's disease and other neurodegenerative diseases, amyloidosis associated with chronic hemodialysis, diabetes type II, insulinoma, the amyloidosis of the prion diseases, (transmissible spongiform encephalopathies prion diseases), Creutzfeldt-Jakob disease, Gerstmann-Straussler syndrome, Kuru, and scrapie, the amyloidosis associated with carpal tunnel syndrome, senile cardiac amyloidosis, familial amyloidotic polyneuropathy, and the amyloidosis associated with endocrine tumors, especially Alzheimer's disease and type 2 diabetes.

In aspects of the invention, diseases that can be treated and/or prevented using a cyclohexanehexol and a NSAID, in particular R-NSAID, compositions, conjugates, and methods of the invention include conditions of the central or peripheral nervous system or a systemic organ that result in the deposition of proteins, protein fragments, and peptides in beta-pleated sheets, fibrils, and/or aggregates or oligomers. In particular aspects, the disease is Alzheimer's disease, presenile and senile forms; amyloid angiopathy; mild cognitive impairment; Alzheimer's disease-related dementia (e.g., vascular or Alzheimer dementia); tauopathy (e.g., argyrophilic grain dementia, corticobasal degeneration, dementia pugilistica, diffuse neurofibrillary tangles with calcification, frontotemporal dementia with parkinsonism, Prion-related disease, Hallervorden-Spatz disease, myotonic dystrophy, Niemann-Pick disease type C, non-Guamanian Motor Neuron disease with neurofibrillary tangles, Pick's disease, postencephalitic parkinsonism, cerebral amyloid angiopathy, progressive subcortical gliosis, progressive supranuclear palsy, subacute sclerosing panencephalitis, and tangle only dementia), alpha-synucleinopathy (e.g., dementia with Lewy bodies, multiple system atrophy with glial cytoplasmic inclusions, Shy-Drager syndrome, spinocerebellar ataxia (e.g., Dentatorubral-Pallidoluysian atrophy (DRPLA) or Machado-Joseph Disease); striatonigral degeneration; olivopontocerebellar atrophy, neurodegeneration with brain iron accumulation type I, olfactory dysfunction; Parkinson's disease (e.g., familial or non-familial); Amyotrophic Lateral Sclerosis; Spastic paraplegia (e.g., associated with defective function of chaperones and/or triple A proteins); Huntington's Disease, spinocerebellar ataxia, Freidrich's Ataxia; neurodegenerative diseases associated with intracellular and/or intraneuronal aggregates of proteins with polyglutamine, polyalanine or other repeats arising from pathological expansions of tri- or tetra-nucleotide elements within corresponding genes; cerebrovascular diseases; Down's syndrome; multiple sclerosis, head trauma with post-traumatic accumulation of amyloid beta peptide; Prion related disease (Creutzfeldt-Jakob disease, Gerstmann-Straussler-Scheinker disease, and variant Creutzfeldt-Jakob disease); Familial British Dementia; Familial Danish Dementia; Presenile Dementia with Spastic Ataxia; Cerebral Amyloid Angiopathy, British Type; Presenile Dementia With Spastic Ataxia Cerebral Amyloid Angiopathy, Danish Type; Familial encephalopathy with neuroserpin inclusion bodies (FENIB); Amyloid Polyneuropathy (e.g., senile amyloid polyneuropathy or systemic Amyloidosis); Inclusion Body myositis due to amyloid beta peptide; Familial and Finnish Type Amyloidosis; Systemic amyloidosis associated with multiple myeloma; Familial Mediterranean Fever; Alexander disease; Alper's disease; ataxia telangiectasia, Canavan disease, Cockayne syndrome, corticobasal degeneration, Batten disease, HIV-asssociated dementia, Kennedy's disease, Krabbe disease, Pelizaeus-Merzbacher Disease, primary lateral sclerosis, Refsum's disease, Sandhoff disease, spinal muscular atrophy, Steele-Richardson-Olszewski disease, Tabes dorsalis, chronic infections and inflammations; and Type 2 Diabetes Mellitus associated with islet amyloid polypeptide (IAPP).

In selected aspects of the invention, in particular combination therapies, the disease is a neuronal disorder (e.g., Alzheimer's disease, Down Syndrome, Parkinson's disease, Chorea Huntington, pathogenic psychotic conditions, schizophrenia, impaired food intake, sleep-wakefulness, impaired homeostatic regulation of energy metabolism, impaired autonomic function, impaired hormonal balance, impaired regulation of body fluids, hypertension, fever, sleep dysregulation, anorexia, anxiety related disorders including depression, seizures including epilepsy, drug withdrawal and alcoholism, disorders including cognitive dysfunction and dementia).

In certain selected aspects of the invention, the disease is a neurodegenerative disease or neurodegenerative disorder including such diseases and impairments as Alzheimer's disease, dementia, MCI, Huntington's disease, multiple sclerosis, Parkinson's disease, amyotrophic lateral sclerosis, epilepsy, Pick's disease, and other similar diseases and disorders disclosed herein.

A cyclohexanehexol and a NSAID, in particular R-NSAID, composition, or conjugate described herein may also act to inhibit or prevent α-synuclein/NAC fibril formation, inhibit or prevent α-synuclein/NAC fibril growth, and/or cause disassembly, disruption, and/or disaggregation of preformed α-synuclein/NAC fibrils and α-synuclein/NAC-associated protein deposits. Examples of synuclein diseases or synucleinopathies suitable for treatment with a cyclohexanehexol and a NSAID, in particular R-NSAID, composition, or conjugate described herein are diseases associated with the formation, deposition, accumulation, or persistence of synuclein fibrils, especially α-synuclein fibrils, including without limitation Parkinson's disease, familial Parkinson's disease, Lewy body disease, the Lewy body variant of Alzheimer's disease, dementia with Lewy bodies, multiple system atrophy, olivopontocerebellar atrophy, neurodegeneration with brain iron accumulation type I, olfactory dysfunction, and the Parkinsonism-dementia complex of Guam.

In certain selected aspects of the invention, the disease is a Motor Neuron Disease associated with filaments and aggregates of neurofilaments and/or superoxide dismutase proteins, the Spastic paraplegia associated with defective function of chaperones and/or triple A proteins, or a spinocerebellar ataxia such as DRPLA or Machado-Joseph Disease.

In other selected aspects of the invention, the disease is a Prion Disease including Creutzfeldt-Jakob disease, Gerstmann-Strausller-Scheinfer disease, and variant Creutzfeldt-Jakob disease, or an Amyloid Polyneuropathy including senile amyloid polyneuropathy or systemic amyloidosis.

In embodiments of the invention, the disease is Alzheimer's disease or Parkinson's disease including familial and non-familial types. In particular embodiments of the invention, the disease is Alzheimer's disease.

Alzheimer's disease (AD) affects about 4.5 million men and women in the United States alone. The incidence of Alzheimer's disease increases with age, and it affects up to 50 percent of people older than 85, with the risk generally increasing with age. Thus, one of the risk factors to consider when assessing whether a patient or patient population is a suitable host for the treatment and/or prevention of Alzheimer's disease is age. Of course signs or symptoms of the disease are an even better predictor. However, in many cases in people with Alzheimer's disease, changes in the brain may begin 10 to 20 years before any visible signs of dementia or symptoms appear. Thus, early treatment, even before the onset of visible signs, would positively affect the treatment and/or prevention of Alzheimer's disease, or would at least delay the effects thereof, or decrease their severity.

There are many diagnostic tests available to practitioners that help assess a patient's chance of having and/or developing Alzheimer's disease. These tests include, for example, tests known in the field as Mini-mental State Examination (MMSE), Clock Drawing Test, Clinical Dementia Rating (CDR) scale, Mini-Mental State Examination (MMSE), Functional Assessment, e.g., using a Functional Assessment Staging (FAST) scale, Alzheimer's Disease Assessment Scale-Cognitive Subscale (ADAS-Cog), among other tests known in the art. Many tests focus on assessment of memory, problem-solving, vision-motor coordination, attention, and abstract thinking, such as performing simple calculations in one's head. Doctors use a variety of assessments and laboratory measurements to make a diagnosis.

Brains of individuals with AD exhibit characteristic lesions termed senile (or amyloid) plaques, amyloid angiopathy (amyloid deposits in blood vessels) and neurofibrillary tangles. Large numbers of these lesions, particularly amyloid plaques and neurofibrillary tangles, are generally found in several areas of the human brain important for memory and cognitive function in patients with AD. Smaller numbers of these lesions in a more restricted anatomical distribution are also found in the brains of most aged humans who do not have clinical AD. Amyloid plaques and amyloid angiopathy also characterize the brains of individuals with Trisomy 21 (Down's Syndrome) and Hereditary Cerebral Hemorrhage with Amyloidosis of the Dutch-Type (HCHWA-D). Detection of such lesions, using MRI, CT, PET, SPECT, etc., is also useful in diagnosing AD.

In certain aspects of the invention, the disease may be characterized by an inflammatory process due to the presence of macrophages by an amyloidogenic protein or peptide. A method of the invention may involve inhibiting macrophage activation and/or inhibiting an inflammatory process. A method may comprise decreasing, slowing, ameliorating, or reversing the course or degree of macrophage invasion or inflammation in a patient.

A disease may be a condition that is associated with a molecular interaction that can be disrupted or dissociated with a cyclohexanehexol. “A molecular interaction that can be disrupted or dissociated with a cyclohexanehexol” includes an interaction comprising an amyloid protein and a protein or glycoprotein. An interaction comprising an amyloid protein includes an amyloid protein-amyloid protein interaction, amyloid-proteoglycan interaction, amyloid-proteoglycan/glycosaminoglycan (GAG) interaction and/or amyloid protein-glycosaminoglycan interaction. An interacting protein may be a cell surface, secreted or extracellular protein. Examples of such diseases include infectious diseases caused by bacteria, viruses, prions and fungi. Examples of such disorders and/or diseases are those associated with pathogens including Herpes simplex virus, Pseudorabies virus, human cytomegalovirus, human immunodeficiency virus, Bordetella pertussis, Chlamydia trachomatis, Haemophilus influenzae, Helicobacter pylori, Borrelia burgdorferi, Neisseria gonorrhoeae, Mycobacterium tuberculosis, Staphylococcus aureus, Streptococcus mutans, Streptococcus suis, Plasmodium falciparum, Leishmania amazonensi, Trypanozoma cruzi, Listeria monocytogenes, Mycoplasma pneumoniae, enterotoxigenic E. coli, uropathogenic E. coli, and Pseudomonas aeruginosa.

### Compositions and Conjugates

One or more cyclohexanehexol, especially a scyllo-inositol compound, and one or more NSAID, in particular R-NSAID, may be formulated into a pharmaceutical composition for administration to a subject. A pharmaceutical composition may be a formulation including without limitation pills, tablets, caplets, soft and hard gelatin capsules, lozenges, sachets, cachets, vegicaps, liquid drops, elixirs, suspensions, emulsions, solutions, syrups, aerosols (as a solid or in a liquid medium) suppositories, sterile injectable solutions, sustained release formulation, and/or sterile packaged powders, which comprise a cyclohexanehexol, especially a scyllo-inositol compound and a NSAID, in particular R-NSAID. Various delivery systems are known and can be used to administer a composition of the invention, e.g. encapsulation in liposomes, microparticles, microcapsules, and the like.

Pharmaceutical compositions of the present invention or fractions thereof typically comprise suitable pharmaceutically acceptable carriers, excipients, and vehicles selected based on the intended form of administration, and consistent with conventional pharmaceutical practices. Particular compositions of the invention may contain a cyclohexanehexol, especially a scyllo-inositol compound and a NSAID, in particular R-NSAID, that are pure or substantially pure. Suitable pharmaceutical carriers, excipients, and vehicles are described in the standard text, Remington: The Science and Practice of Pharmacy (21^{st} Edition. 2005, University of the Sciences in Philadelphia (Editor), Mack Publishing Company), and in The United States Pharmacopeia: The National Formulary (USP 24 NF 19) published in 1999.

In aspects of the invention, a pharmaceutical composition is provided for oral administration of one or more cyclohexanehexol, especially a scyllo-inositol compound, and one or more NSAID, in particular R-NSAID, for treatment of a disease. In a particular aspect, a stable oral pharmaceutical composition for treatment of a disease characterized by abnormal protein folding and/or aggregation, and/or amyloid formation, deposition, accumulation, or persistence (e.g., Alzheimer's disease) is provided comprising a substantially pure scyllo-inositol compound of the formula I, II, III, IV, V or VI, preferably a compound of the formula V or VI, and a NSAID, in particular R-NSAID selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-etodolac and R-benoxaprofen.

An oral pharmaceutical composition may be in the form of a capsule or tablet and the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the active components may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof Compositions as described herein can further comprise wetting or emulsifying agents, or pH buffering agents.

In other aspects of the invention, a pharmaceutical composition is provided for parenteral administration of one or more cyclohexanehexol, especially a scyllo-inositol compound, and one or more NSAID, in particular R-NSAID. A parenteral formulation may include aqueous or non-aqueous solutions (e.g. water, isotonic saline, isotonic glucose solution, buffer solution, or other conventional solvents), syrups, aqueous or oil suspensions and emulsions with edible oil such as cottonseed oil, coconut oil, almond oil, or peanut oil. A composition intended for parenteral administration may also include conventional additives such as stabilizers, buffers, preservatives, or dispersing or suspending agents, for example, antioxidants such as methylhydroxybenzoate or similar additives.

Compositions of the invention can be formulated as pharmaceutically acceptable salts as described herein.

In aspects of compositions of the invention the ratio of a cyclohexanehexol to NSAID is selected to augment the activity of the scyllo-inositol compound and/or NSAID. In particular aspects of the compositions of the invention the ratio of a cyclohexanehexol and NSAID, in particular R-NSAID, is from about 1:1 to 1:110, 1:1 to 1:100, 1:1 to 1:75, 1:1 to 1:50, 1:1 to 1:25, 1:1 to 1:10, 1:1 to 1:5, and 1:1. In other aspects the ratio of NSAID, in particular R-NSAID, to a cyclohexanehexol is from about 1:1 to 1:110, 1:1 to 1:100, 1:1 to 1:75, 1:1 to 1:50, 1:1 to 1:25, 1:1 to 1:10, and 1:1 to 1:5.

The invention further provides a conjugate comprising a cyclohexanehexol, especially a scyllo-inositol compound linked to a NSAID, in particular R-NSAID, isolated covalent conjugates, and compositions comprising covalent conjugates of the invention. Conjugates of a cyclohexanehexol, especially a scyllo-inositol compound, and NSAID, in particular R-NSAID, may be conjugated or linked with an intermediate spacer or linker. A suitable spacer or linker may be a mono- or disaccharide, an amino acid(s), a sulfate, a succinate, an acetate, or an oligomeric polymeric spacer or linker comprising one or more of such moieties.

The invention also provides methods of preparing covalent conjugates that preferably result in conjugates with improved pharmacokinetic properties, biological activity, and beneficial effects. The methods comprise incubating or reacting the cyclohexanehexol with the NSAID, in particular R-NSAID, under conditions that allow formation of a covalent linkage between the two compounds. The invention therefore contemplates a process for preparing a covalent conjugate comprising a cyclohexanehexol covalently bonded or linked to a NSAID, in particular R-NSAID, the process comprising: incubating or reacting the cyclohexanehexol with a NSAID, in particular R-NSAID, under conditions and at a pH and for a time sufficient for formation of a covalent bond or linkage between the cyclohexanehexol and NSAID, in particular R-NSAID; and isolating the covalent conjugate. The above process for preparing a conjugate comprising a cyclohexanehexol and a NSAID, in particular R-NSAID, can provide a conjugate with a substantial amount of a cyclohexanehexol covalently linked to the NSAID, in particular R-NSAID.

The invention further relates to a pharmaceutical formulation of a substantially pure covalent conjugate comprising a cyclohexanehexol covalently linked to a NSAID, in particular R-NSAID, which provides beneficial effects preferably sustained beneficial effects compared to the cyclohexanehexol or a NSAID, in particular R-NSAID, alone. In an embodiment, a pharmaceutical formulation is provided consisting essentially of covalent conjugates comprising a cyclohexanehexol covalently linked without an intermediate spacer or linker to a NSAID, in particular R-NSAID. In another embodiment, a pharmaceutical formulation is provided consisting essentially of covalent conjugates comprising a cyclohexanehexol covalently linked with an intermediate spacer or linker to a NSAID, in particular R-NSAID.

Compounds, compositions or conjugates of the invention may be sterilized by, for example, filtration through a bacteria retaining filter, addition of sterilizing agents to the compounds, compositions or conjugates, irradiation of the compounds, compositions, or conjugates, or heating the compounds, compositions or conjugates. Alternatively, the compounds, compositions, or conjugates may be provided as sterile solid preparations, e.g. lyophilized powder, that are readily dissolved in sterile solvent immediately prior to use.

After pharmaceutical compositions or conjugates have been prepared, they can be placed in an appropriate container and labeled for treatment of an indicated condition. For administration of a composition of the invention, such labeling would include amount, frequency, and method of administration.

In combination treatments of the invention, a cyclohexanehexol, in particular a scyllo-inositol compound, may be in a form suitable for administration as a dietary supplement. A supplement may optionally include inactive ingredients such as diluents or fillers, viscosity-modifying agents, preservatives, flavorings, colorants, or other additives conventional in the art. By way of example only, conventional ingredients such as beeswax, lecithin, gelatin, glycerin, caramel, and carmine may be included. The dietary supplement may be provided as a liquid dietary supplement (e.g., a dispensable liquid) or alternatively the compositions may be formulated as granules, capsules or suppositories. The liquid supplement may include a number of suitable carriers and additives including water, glycols, oils, alcohols, flavoring agents, preservatives, coloring agents and the like. In capsule, granule or suppository form, the compositions of the present invention are formulated in admixture with a pharmaceutically acceptable carrier. A dietary supplement may be formulated as a beverage, but may be formulated in granule, capsule or suppository form. A supplement may be presented in the form of a softgel which is prepared using conventional methods. A softgel typically includes a layer of gelatin encapsulating a small quantity of the supplement. A supplement may also be in the form of a liquid-filled and sealed gelatin capsule, which may be made using conventional methods.

To prepare a dietary supplement comprising a cyclohexanehexol, in particular a scyllo-inositol compound, in capsule, granule or suppository form, one or more cyclohexanehexol, in particular scyllo-inositol compound, may be intimately admixed with a pharmaceutically acceptable carrier according to conventional formulation techniques. For solid oral preparations such as capsules and granules, suitable carriers and additives such as starches, sugars, diluents, granulating agents, lubricants, binders, disintegrating agents and the like may be included.

The invention also provides kits. In an aspect, a kit comprises a cyclohexanehexol and a NSAID, in particular R-NSAID, or a pharmaceutical composition or conjugate of the invention. The kit can be a package which houses a container which contains a composition or conjugate of the invention and also houses instructions for administering the composition or conjugates to a subject. A kit may contain a single dosage form or it may contain two dosage forms, i.e. one for each compound to be administered. In an aspect, the kit comprises a fixed ratio dosage of a cyclohexanehexol and a NSAID, in particular R-NSAID. The invention further relates to a commercial package a cyclohexanehexol and a NSAID, in particular R-NSAID, or compositioinn according to the present invention together with instructions for simultaneous, separate or sequential use.

In aspects of the invention, a pharmaceutical pack or kit is provided comprising one or more containers filled with one or more of the active ingredients of a pharmaceutical composition or conjugate of the invention, in particular to provide a beneficial effect, more particularly a sustained beneficial effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

### Applications

The invention contemplates the use of one or more cyclohexanehexol, and one or more NSAID, in particular R-NSAID, or a composition or conjugate of the invention for treating a disease, in particular preventing, and/or ameliorating disease severity, disease symptoms, and/or periodicity of recurrence of a disease disclosed herein. The invention also contemplates preventing and/or treating diseases in mammals using a combination of one or more cyclohexanehexol and one or more NSAID, in particular R-NSAID, or a composition, conjugate or treatment of the invention. The present invention in embodiments may provide a composition comprising one or more cyclohexanehexol and one or more NSAID, in particular R-NSAID, that provides beneficial effects including greater solubility, stability, efficacy, potency, and/or utility, in particular greater solubility and stability.

Greater efficacy and potency of a treatment of the invention in some aspects may improve the therapeutic ratio of treatment, reducing untoward side effects and toxicity. Selected methods of the invention may also improve long-standing disease (e.g., Alzheimer's disease) even when treatment is begun long after the appearance of symptoms. Prolonged efficacious treatment may be achieved in accordance with the invention following administration of one or more cyclohexanehexol and a NSAID, in particular R-NSAID, or composition of the invention.

A composition or method of the invention may provide beneficial effects including an improvement or lessening in decline in biochemical disease marker progression, plaque pathology, quality of life indicators or combinations of any disease parameters.

In an aspect, the invention provides a method of preventing or reversing conformationally altered protein assembly or aggregation in an animal that includes introducing one or more cyclohexanehexol and one or more NSAID, in particular R-NSAID, including, their analogs, or derivatives thereof, or a composition or conjugate of the invention, to the conformationally altered protein.

In a further aspect of the invention, a method of preventing or reversing conformationally altered protein assembly or aggregation in an animal is provided that includes introducing one or more cyclohexanehexol, especially a scyllo-inositol compound, and one or more NSAID, in particular R-NSAID, or a composition or conjugate of the invention, to the conformationally altered protein.

In a still further aspect of the invention, a method of treating conformationally altered protein assembly or aggregation in an animal is provided that includes administering a therapeutically effective amount of one or more cyclohexanehexol, especially a scyllo-inositol compound, and one or more NSAID, in particular R-NSAID, or compositions or conjugates of the invention.

In an aspect, the invention provides a method for amelioriating progression of a disease or obtaining a less severe stage of a disease in a subject suffering from such disease (e.g. Alzheimer's disease) comprising administering a therapeutically effective amount of one or more cyclohexanehexol and one or more NSAID, in particular R-NSAID, or pharmaceutically acceptable salts thereof, or a composition or conjugate of the invention.

In another aspect, the invention relates to a method of delaying the progression of a disease (e.g. Alzheimer's disease) comprising administering a therapeutically effective amount of one or more cyclohexanehexol and one or more NSAID, in particular R-NSAID, pharmaceutically acceptable salts thereof, or a composition or conjugate of the invention.

In a further aspect, the invention relates to a method of increasing survival of a subject suffering from a disease comprising administering a therapeutically effective amount of one or more cyclohexanehexol and one or more NSAID, in particular R-NSAID, pharmaceutically acceptable salts thereof, or a composition or conjugate of the invention.

In an aspect, the invention relates to a method of improving the lifespan of a subject suffering from a disease (e.g., Alzheimer's disease) comprising administering a therapeutically effective amount of one or more cyclohexanehexol and one or more NSAID, in particular R-NSAID, pharmaceutically acceptable salts thereof, or a composition or conjugate of the invention.

Aspects of the invention provide improved methods and compositions for use of one or more cyclohexanehexol and one or more NSAID, in particular R-NSAID, for sustained treatment of a disease (e.g., Alzheimer's disease). The present invention in an embodiment provides a composition comprising one or more cyclohexanehexol and one or more NSAID, in particular R-NSAID, that achieve greater efficacy, potency, and utility. For example, the greater efficacy can be shown by improving or reversing cognitive decline and/or survival in Alzheimer's disease with treatment resulting in sustained improvement and/or increased survival after ceasing treatment.

In an aspect, the invention provides a method of improving memory of a healthy subject or the memory of a subject with age impaired memory by administering an effective amount of one or more cyclohexanehexol and one or more NSAID, in particular R-NSAID, or a composition or conjugate of the invention.

In another aspect, the present invention relates to a method for improving memory, especially short-term memory and other mental dysfunction associated with the aging process comprising administering an effective amount of one or more cyclohexanehexol and one or more NSAID, in particular R-NSAID, or pharmaceutically acceptable salts thereof, or a composition or conjugate of the invention.

In an embodiment, a method is provided for treating a mammal in need of improved memory, wherein said mammal has no diagnosed disease, disorder, infirmity or ailment known to impair or otherwise diminish memory, comprising the step of administering to the mammal an effective memory-improving amount of one or more cyclohexanehexol and one or more NSAID, in particular R-NSAID, pharmaceutically acceptable salts thereof, or a dietary supplement comprising one or more cyclohexanehexol or a nutraceutically acceptable derivative thereof, and one or more NSAID, in particular R-NSAID.

In another aspect of the invention, a method is provided for treating in a subject a condition of the central or peripheral nervous system or systemic organ associated with a disorder in protein folding or aggregation, or amyloid formation, deposition, accumulation, or persistence, comprising administering to the subject a therapeutically effective amount of one or more cyclohexanehexol and one or more NSAID, in particular R-NSAID, or pharmaceutically acceptable salts thereof, or a composition or conjugate of the invention.

The invention has particular applications in treating a disease characterized by amyloid deposition, in particular an amyloidoses, more particularly Alzheimer's disease. Thus, the invention relates to a method of treatment comprising administering a therapeutically effective amount of one or more cyclohexanehexol and one or more NSAID, in particular R-NSAID, pharmaceutically acceptable salts thereof, or a composition or conjugate of the invention. Therapeutically effective amounts of the compounds can produce beneficial effects, preferably sustained beneficial effects, upon administration to a subject with symptoms of a disease characterized by amyloid deposition, more particularly Alzheimer's disease. In an embodiment, beneficial effects are evidenced by one or more of the following: disruption of aggregated Aβ or Aβ oligomers, increased or restored long term potentiation, and/or maintenance of or increased synaptic function, and/or, reduced cerebral accumulation of Aβ, deposition of cerebral amyloid plaques, soluble Aβ oligomers in the brain, glial activity, inflammation, and/or cognitive decline.

In an aspect, the invention provides a method involving administering to a subject a therapeutic compound of one or more cyclohexanehexol, especially a scyllo-inositol compound, and one or more NSAID, in particular R-NSAID, or pharmaceutically acceptable salts thereof, or a composition or conjugate of the invention which inhibit amyloid formation, deposition, accumulation and/or persistence, and/or which cause dissolution/disruption of pre-existing amyloid. Thus, a combination of a cyclohexanehexol(s) and NSAID(s), in particular R-NSAID(s) or compositions or conjugates of the invention may be used for inhibiting amyloidosis in disorders in which amyloid deposition occurs.

In another aspect, the invention provides a method for treating in a subject a condition associated with an amyloid interaction that can be disrupted or dissociated with one or more cyclohexanehexol, especially a scyllo-inositol compound, comprising administering to the subject a therapeutically effective amount of one or more cyclohexanehexol, especially scyllo-inositol compound, and one or more NSAID, in particular R-NSAID, or pharmaceutically acceptable salts thereof, or a composition or conjugate of the invention.

In an aspect, the invention provides a method for preventing, reversing, reducing or inhibiting amyloid protein assembly, enhancing clearance of amyloid deposits, or slowing deposition of amyloid deposits in a subject comprising administering a therapeutically effective amount of one or more cyclohexanehexol, especially scyllo-inositol compound, and one or more NSAID, in particular R-NSAID, or pharmaceutically acceptable salts thereof, or a composition or conjugate of the invention.

In an aspect, the invention provides a method for preventing, reversing, reducing or inhibiting amyloid fibril formation, organ specific dysfunction (e.g., neurodegeneration), or cellular toxicity in a subject comprising administering to the subject a therapeutically effective amount of one or more cyclohexanehexol, especially a scyllo-inositol compound, and one or more NSAID, in particular R-NSAID, pharmaceutically acceptable salts thereof, or a composition or conjugate of the invention.

In another embodiment, the invention provides a therapeutic method which comprises identifying a patient diagnosed with a neurodegenerative disorder (such as Alzheimer's disease or MCI) and treating the patient with an effective amount of one or more cyclohexanehexol, especially a scyllo-inositol compound, and one or more NSAID, in particular R-NSAID, or a composition or conjugate of the invention.

A patient can be diagnosed with a disease such as a neurodegenerative disorder using a suitable combination of tests and observations. For example, criteria that indicate a likelihood of mild to moderate Alzheimer's disease include a score of about 15 to about 26 on the MMSE test or a decline in cognitive function.

In a further embodiment, the invention provides a prophylactic method which comprises identifying a patient in need of prophylaxis against a neurodegenerative disorder (such as Alzheimer's disease or MCI) or the worsening of one or more symptoms of such disorder, and treating the patient with an effective amount of one or more cyclohexanehexol, especially a scyllo-inositol compound, and one or more NSAID, in particular R-NSAID or a composition or conjugate of the invention.

Patients in need of prophylaxis can be assessed by monitoring assayable disease markers (e.g. scyllo-inositol), detection of genes conferring a predisposition to the disease, and other risk factors such as age, diet, and other associated diseases. A patient desiring prophylaxis against a disease (e.g., Alzheimer's disease) or the worsening of one or more symptoms of such a disease can be treated with a combination, conjugate or method disclosed herein prior to onset of symptoms of the disease or just at the beginning stages of disease

In an aspect, the invention provides a method for increasing or maintaining synaptic function in a subject comprising administering a therapeutically effective amount of one or more cyclohexanehexol, especially a scyllo-inositol compound, and one or more NSAID, in particular R-NSAID, pharmaceutically acceptable salts thereof, or a composition or conjugate of the invention.

In an aspect the invention provides a method for treating mild cognitive impairment (MCI) comprising administering a therapeutically effective amount of one or more cyclohexanehexol, especially scyllo-inositol compound, and one or more NSAID, in particular R-NSAID, or pharmaceutically acceptable salts thereof, or a composition or conjugate of the invention.

In an embodiment, the invention provides a method of reducing or reversing amyloid deposition and neuropathology after the onset of cognitive deficits and amyloid plaque neuropathology in a subject comprising administering to the subject a therapeutically effective amount of one or more cyclohexanehexol, especially a scyllo-inositol compound, and one or more NSAID, in particular R-NSAID, pharmaceutically acceptable salts thereof, or a composition or conjugate of the invention.

In another embodiment, the invention provides a method of reducing or reversing amyloid deposition and neuropathology after the onset of cognitive deficits and amyloid plaque neuropathology in a subject comprising administering to the subject an amount of one or more cyclohexanehexol, especially a scyllo-inositol compound, and one or more NSAID, in particular R-NSAID, pharmaceutically acceptable salts thereof, or a composition or conjugate of the invention, effective to reduce or reverse amyloid deposition and neuropathology after the onset of cognitive deficits and amyloid plaque neuropathology.

Particular aspects of the invention relate to a method for treating Alzheimer's disease comprising contacting Aβ, Aβ aggregates, or Aβ oligomers in particular Aβ40 or Aβ40 aggregates or oligomers and/or Aβ42 or Aβ42 aggregates or oligomers, in a subject with a therapeutically effective amount of one or more cyclohexanehexol, especially scyllo-inositol compound, and one or more NSAID, in particular R-NSAID, or a composition or conjugate of the invention.

In an embodiment, the invention provides a method for treating Alzheimer's disease by providing one or more cyclohexanehexol, especially scyllo-inositol compound, and a

NSAID, in particular R-NSAID, or a composition or conjugate of the invention, in an amount sufficient to disrupt aggregated Aβ or Aβ oligomers for a prolonged period following administration.

In another embodiment, the invention provides a method for treating Alzheimer's disease in a patient in need thereof which includes administering to the individual one or more cyclohexanehexol, especially scyllo-inositol compound, and one or more NSAID, in particular R-NSAID, or a composition or conjugate of the invention, in a dose(s) sufficient to increase or restore long term potentiation and/or maintain synaptic function. In a further embodiment, the invention provides a method for treating Alzheimer's disease comprising administering, preferably orally or systemically, amounts of a cyclohexanehexol, especially scyllo-inositol compound, and a NSAID, in particular R-NSAID, or a composition or conjugare of the invention, to a mammal, to reduce cerebral accumulation of Aβ, deposition of cerebral amyloid plaques, soluble Aβ oligomers in the brain, glial activity, and/or inflammation for a prolonged period following administration.

The invention in an embodiment provides a method for treating Alzheimer's disease, the method comprising administering to a mammal in need thereof one or more cyclohexanehexol, especially scyllo-inositol compound, and one or more NSAID, in particular R-NSAID, or a composition or conjugate of the invention, in an amount(s) sufficient to reduce cognitive decline, especially for a prolonged period following administration, thereby treating the Alzheimer's disease.

The invention in an embodiment provides a method for treating Alzheimer's disease, the method comprising administering to a mammal in need thereof one or more cyclohexanehexol, especially scyllo-inositol compound, and one or more NSAID, in particular R-NSAID, or a composition or conjugate of the invention, in an amount(s) sufficient to increase or maintain synaptic function, especially for a prolonged period following administration, thereby treating the Alzheimer's disease.

The invention also provides a method for preventing and/or treating Alzheimer's disease, the method comprising administering to a mammal in need thereof one or more cyclohexanehexol, especially scyllo-inositol compound, and one or more NSAID, in particular R-NSAID, or a composition or conjugate of the invention, in an amount(s) sufficient to disrupt aggregated Aβ or Aβ oligomers for a prolonged period following administration; and determining the amount of aggregated Aβ or Aβ oligomers, thereby treating the Alzheimer's disease. The amount of aggregated Aβ or Aβ oligomers may be measured using an antibody specific for Aβ or a scyllo-inositol labeled with a detectable substance such as a radioisotope (e.g., ³H, ¹⁴C, ³⁵S, ¹²⁵I, ¹³¹I), fluorescent label (e.g., FITC, rhodamine, lanthanide phosphors), luminescent label such as luminal, enzymatic label (e.g., horseradish peroxidase, beta-galactosidase, luciferase, alkaline phosphatase, acetylcholinesterase), or biotinyl group.

In embodiments of the invention a therapeutically effective amount of a compound of the formula I, II, III, IV, V or VI and a NSAID, in particular R-NSAID are utilized in the treatment of Alzheimer's disease. Such treatment may be effective for retarding the degenerative effects of Alzheimer's disease, including specifically, but not exclusively, deterioration of the central nervous system, loss of mental facilities, loss of short term memory, and disorientation.

In particular embodiments of the invention, where the disease is Alzheimer's disease, beneficial effects of a cyclohexanehexol and NSAID (preferably scyllo-inositol and R-flurbiprofen), a composition, conjugate or treatment of the invention can manifest as at least one, two, three, four, five, six, seven, eight, nine, ten, twelve, thirteen, fourteen, fifteen, or all of the following, in particular five or ten or more, more particularly fifteen or more of the following:
a) Prevention, increase or restoration of long term potentiation relative to the level in the absence of a cyclohexanehexol, especially scyllo-inositol compound, and/or NSAID, in particular R-NSAID, especially R-flurbiprofen, after administration to a subject with symptoms of Alzheimer's disease. In aspects of the invention a cyclohexanehexol, especially a scyllo-inositol compound, and a NSAID, in particular R-NSAID, induce at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% increase in long term potentiation in a subject.
b) Prevention, increase or maintenance of synaptic function relative to the level of synaptic function in the absence of a cyclohexanehexol, especially a scyllo-inositol compound, and/or NSAID, in particular R-NSAID, especially R-flurbiprofen, after administration to a subject with symptoms of Alzheimer's disease. In aspects of the invention a cyclohexanehexol, especially scyllo-inositol compound, and a NSAID, in particular R-NSAID, especially R-flurbiprofen, induce at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, 99%, 100%, 125%, 150%, 175% or 200% increase in synaptic function in a subject.
c) An increase in synaptophysin. In aspects of the invention there is at least about a 2%, 5%, 10%, 15%, 20%, 30%, 40%, 50%, 60%, 70%, 80%, 90%, 95%, 99%, 100%, 125%, 150%, 175% or 200% increase in synaptophysin.
d) An increase in synaptophysin reactive boutons and cell bodies. In aspects of the invention there is at least about a 2%, 5%, 10%, 15%, 20%, 30%, 40%, 50%, 60%, 70%, 80%, 90%, 95%, 99%, 100%, 125%, 150%, 175% or 200%, more particularly about a 100-150% or 140-150% increase in synaptophysin reactive boutons and cell bodies.
e) Prevention, reduction, slowing or an absence of symptoms of inflammation, in particular an Aβ-induced inflammatory response, after administration to a subject with symptoms of Alzheimer's disease.
f) Prevention or reduction in cerebral accumulation of amyloid β relative to the levels measured in the absence of a cyclohexanehexol, especially scyllo-inositol compound, and a NSAID, in particular R-NSAID, in subjects with symptoms of Alzheimer's disease. In aspects of the invention, the combination induces at least about a 2%, 5%, 10%, 15%, 20%, 30%, 40%, 50%, 60%, 70%, 80%, or 90% decrease in cerebral accumulation of amyloid β.
g) Prevention or reduction in deposition of cerebral amyloid plaques, relative to the levels measured in the absence of a cyclohexanehexol, especially scyllo-inositol compound, and a NSAID, in particular R-NSAID, especially R-flurbiprofen, in subjects with symptoms of Alzheimer's disease. In aspects of the invention, the combination induces at least about a 2%, 5%, 10%, 15%, 20%, 30%, 40%, 50%, 60%, 70%, 80%, or 90% decrease in deposition of cerebral amyloid plaques.
h) A reduction in plaque number. In aspects of the invention, the combination of a cyclohexanehexol, especially scyllo-inositol compound, and a NSAID, in particular R-NSAID, especially R-flurbiprofen, induces at least about a 2%, 5%, 10%, 15%, 20%, 30%, 40%, 50%, 60%, 70%, 80%, or 90% reduction in plaque number. In particular aspects the combination induces a 5-15% or 10-15% reduction in plaque number.
i) A reduction in plaque size. In aspects of the invention, the combination of a cyclohexanehexol, especially scyllo-inositol compound, and a NSAID, in particular R-NSAID, especially R-flurbiprofen, induces at least about a 2%, 5%, 10%, 15%, 20%, 30%, 40%, 50%, 60%, 70%, 80%, or 90% reduction in plaque size. In particular aspects the compounds induce a 5-15% or 10-15% reduction in plaque size.
j) A reduction in percent area of the brain covered in plaques. In aspects of the invention, the combination of a cyclohexanehexol, especially scyllo-inositol compound, and a NSAID, in particular R-NSAID, especially R-flurbiprofen, induces at least about a 2%, 5%, 10%, 15%, 20%, 30%, 40%, 50%, 60%, 70%, 80%, or 90% reduction in percent area of the brain covered in plaques. In particular aspects the combination induces a 5-15% or 10-15% reduction in percent area of the brain covered in plaques.
k) A reduction in soluble Aβ oligomers in the brain, relative to the levels measured in the absence of a combination of a cyclohexanehexol, especially scyllo-inositol compound, and a NSAID, in particular R-NSAID, especially R-flurbiprofen, in subjects with symptoms of Alzheimer's disease. In aspects of the invention, the combination induces at least about a 2%, 5%, 10%, 15%, 20%, 30%, 40%, 50%, 60%, 70%, 80%, or 90% decrease in soluble Aβ oligomers.
l) A reduction in brain levels of Aβ40. In aspects of the invention, a combination of a cyclohexanehexol, especially scyllo-inositol compound, and a NSAID, in particular R-NSAID, especially R-flurbiprofen, induces at least about a 2%, 5%, 10%, 15%, 20%, 30%, 40%, 50%, 60%, 70%, 80%, or 90% reduction in Aβ40. In particular aspects the combination induces a 10-50%, 20-45%, or 25-35% reduction in brain levels of Aβ40.
m) A reduction in brain levels of A1342. In aspects of the invention, a combination of a cyclohexanehexol, especially scyllo-inositol compound, and a NSAID, in particular R-NSAID, especially R-flurbiprofen, induces at least about a 2%, 5%, 10%, 15%, 20%, 30%, 40%, 50%, 60%, 70%, 80%, or 90% reduction in Aβ42. In particular aspects the combination induces a 10-50%, 15-40%, or 20-25% reduction in brain levels of Aβ42.
n) A reduction in glial activity in the brain, relative to the levels measured in the absence of a combination of a cyclohexanehexol, especially scyllo-inositol compound, and a NSAID, in particular R-NSAID, especially R-flurbiprofen, in subjects with symptoms of Alzheimer's disease. Preferably, the combination induces at least about a 2%, 5%, 10%, 15%, 20%, 30%, 40%, 50%, 60%, 70%, 80%, or 90% decrease in glial activity
o) Maintenance of synaptic function at about normal for a prolonged period of time, in particular for at least 5 weeks, 6 weeks, 8 weeks, 10 weeks, 12 weeks, 14 weeks, 16 weeks, 20 weeks, 24 weeks, 30 weeks, 40 weeks, 52 weeks, or 78 weeks, more particularly, 2 to 4 weeks, 2 to 5 weeks, 3 to 5 weeks, 2 to 6 weeks, 2 to 8 weeks, 2 to 10 weeks, 2 to 12 weeks, 2 to 16 weeks, 2 to 20 weeks, 2 to 24 weeks, 2 weeks to 12 months, or 2 weeks to 24 months following treatment.
p) A reduction or slowing of the rate of disease progression in a subject with Alzheimer's disease. In particular a reduction or slowing of cognitive decline in a subject with Alzheimer's disease.
q) Prevention, reduction, or slowing cognitive deficits or improvement of cognitive abilities.
r) Prevention, reduction or slowing of amyloid angiopathy.
s) A reduction in accelerated mortality.
t) An increase in survival in a subject with symptoms of Alzheimer's disease.

In aspects of the invention, therapeutic effects of a cyclohexanehexol and NSAID (preferably scyllo-inositol and R-flurbiprofen), a composition, conjugate or treatment of the invention can manifest as (a) and (b); (a), (b) and (c); (a), (b), (e), (0 and (g); (a), (b), (e), (f) through (h); (a), (b), (e), (0 through (i); (a), (b), (e), (f) through (j); (a), (b), (e), (f) through (k); (a), (b), (e), (f) through (l); (a), (b), (e), (f) through (m); (a), (b), (e), (f) through (n); (a), (b), (e), (f) through (o); (a), (b), (e), (f) through (p); (a), (b), (e), (f) through (q); (a), (b), (e), (f) through (r); (a), (b), (e), (0 through (s); (a), (b), (e), (f) through (t); (a) through (d); (a) through (e); (a) through (f); (a) through (g); (a) through (h); (a) through (i); (a) through (j); (a) through (k); (a) through (l); (a) through (m); (a) through (n); (a) through (o); (a) through (p); (a) through (q); (a) through (r); (a) through (s); and (a) through (t).

A cyclohexanehexol, especially scyllo-inositol compound, and a NSAID, in particular R-NSAID, especially R-flurbiprofen, pharmaceutical compositions, conjugates and methods of the invention can be selected that have statistically significant beneficial effects, in particular one or more statistically significant beneficial effects of one or more of, more particularly all of (a) through (t) above. A cyclohexanehexol, especially scyllo-inositol compound, and a NSAID, in particular R-NSAID, especially R-flurbiprofen, pharmaceutical compositions, conjugates and methods of the invention can also be selected that have sustained beneficial effects, in particular statistically significant sustained beneficial effects. In an embodiment, a combination treatment or a pharmaceutical composition is provided with statistically significant sustained beneficial effects, in particular sustained beneficial effects of one or more of (a) through (t) above, comprising a therapeutically effective amount of one or more cyclohexanehexol, especially a scyllo-inositol compound, and a NSAID, in particular R-NSAID, especially R-flurbiprofen. In aspects of the invention, one or more of the beneficial effects provide enhanced therapeutic effects compared with conventional therapies.

The present invention also includes methods of using one or more cyclohexanehexol, especially scyllo-inositol compound, and one or more NSAID, in particular R-NSAID, especially R-flurbiprofen, or compositions of the invention in combination treatments with one or more additional therapeutic agents including without limitation other inhibitors of beta-sheet aggregation/fibrillogenesis/ADDL formation, NMDA antagonists (e.g. memantine, adamantine, amantadine, an adamantine derivative, dextromethorphan, dextrorphan, dizocilpine, ibogaine, ketamine, remacemide, and phencyclidine), anti-oxidants (e.g. Vitamin E), hormones (e.g. estrogens), nutrients and food supplements (e.g. Gingko biloba), statins and other cholesterol lowering drugs (e.g., atorvastatin, simvastatin, lovastatin, fluvastatin, pravastatin, cerivastatin, rosuvastatin, and pitavastatin), acetylcholinesterase inhibitors (e.g. donezepil, tacrine, rivastigmine, and galantamine), β-secretase inhibitors, α-secretase inhibitors, muscarinic agonists (e.g. AF102B (Cevimeline, EVOXAC), AF150(S), and AF267B), GABA-A alpha inverse agonists (see WO00/27382, WO96/25948, WO98/50385), anti-psychotics (e.g. haloperidol, clozapine, olanzapine), anti-depressants including tricyclics and serotonin reuptake inhibitors (e.g. Sertraline and Citalopram Hbr), immunotherapeutics and antibodies to Aβ (e.g. ELAN AN-1792), vaccines, inhibitors of kinases (CDK5, GSK3α, GSK3β) that phosphorylate TAU protein (e.g. lithium chloride), inhibitors of kinases that modulate Aβ production (GSK3α, GSK3β, Rho/ROCK kinases), drugs that upregulate neprilysin (an enzyme which degrades Aβ); drugs that upregulate insulin degrading enzyme (an enzyme which degrades Aβ), and/or agents that are used for the treatment of complications resulting from or associated with a disease, or general medications that treat or prevent side effects. The present invention also includes methods of using the compositions of the invention in combination treatments with one or more additional treatments including without limitation gene therapy and/or drug based approaches to upregulate neprilysin (an enzyme which degrades Aβ), gene therapy and/or drug based approaches to upregulate insulin degrading enzyme (an enzyme which degrades Aβ), or stem cell and other cell-based therapies. The invention further encompasses compositions comprising a combination of active ingredients in accordance with this aspect of the invention.

A combination therapy of the invention optionally with one or more additional therapeutic may provide an additive or synergistic effect, in particular synergistic effect, in one or more of the following: preventing, reducing, reversing or inhibiting Aβ fibril assembly or aggregation, Aβ toxicity, Aβ42 levels, abnormal protein folding, aggregation, amyloid formation, deposition, accumulation or persistence, and/or amyloid lipid interactions, and/or acceleration of disassembly of preformed fibrils. More particularly, a combination therapy of the invention optionally with one or more additional therapeutic may provide a synergistic effect in one or more of the following: disrupting aggregated Aβ or Aβ oligomers; increasing or restoring long term potentiation; maintaining synaptic function; inhibiting, reducing or reversing Aβ-induced progressive cognitive decline and cerebral amyloid plaque pathology; improving cognition; reducing cerebral accumulation of Aβ; reducing deposition of cerebral amyloid plaques; reducing soluble Aβ oligomers (e.g. Aβ42) in the brain and/or body fluids; reducing glial activity; reducing inflammation and/or cognitive decline. A combination therapy of the invention with one or more additional therapeutic may be particularly effective for treating and preventing neurodegenerative disorders especially Alzheimer's disease.

In an aspect, the invention contemplates the use of one or more cyclohexanehexol, especially scyllo-inositol compound, and one or more NSAID, in particular R-NSAID, especially R-flurbiprofen, or a composition comprising one or more cyclohexanehexol, especially scyllo-inositol compound, and one or more NSAID, in particular R-NSAID, especially R-flurbiprofen, for the preparation of a medicament in treating a disease disclosed herein. The invention also contemplates the use of one or more cyclohexanehexol, especially scyllo-inositol compound, and one or more NSAID, in particular R-NSAID, especially R-flurbiprofen, or a composition comprising one or more cyclohexanehexol, especially scyllo-inositol compound, and one or more NSAID, in particular R-NSAID, especially R-flurbiprofen, for the preparation of a medicament for preventing and/or treating diseases. The invention additionally provides uses of one or more cyclohexanehexol, especially scyllo-inositol compound, and one or more NSAID, in particular R-NSAID, especially R-flurbiprofen, or a composition comprising one or more cyclohexanehexol, especially scyllo-inositol compound, and one or more NSAID, in particular R-NSAID, especially R-flurbiprofen, in the preparation of medicaments for the prevention and/or treatment of diseases disclosed herein. The medicaments provide beneficial effects, preferably sustained beneficial effects following treatment. The medicament may be in a form for consumption by a subject such as a pill, tablet, caplet, soft and hard gelatin capsule, lozenge, sachet, cachet, vegicap, liquid drop, elixir, suspension, emulsion, solution, syrup, aerosol (as a solid or in a liquid medium) suppository, sterile injectable solution, and/or sterile packaged powder for inhibition of amyloid formation, deposition, accumulation, and/or persistence, regardless of its clinical setting.

In an embodiment, the invention relates to the use of a therapeutically effective amount of one or more cyclohexanehexol, especially scyllo-inositol compound, and one or more NSAID, in particular R-NSAID, especially R-flurbiprofen, or a composition or conjugate of the invention, for preparation of a medicament for providing therapeutic effects, in particular beneficial effects, preferably sustained beneficial effects, in treating a disease disclosed herein.

In another embodiment the invention provides the use of one or more cyclohexanehexol, especially a scyllo-inositol compound, and one or more NSAID, in particular R-NSAID, especially R-flurbiprofen, or a composition or conjugate of the invention, for the preparation of a medicament for prolonged or sustained treatment of Alzheimer's disease.

In a further embodiment the invention provides the use of one or more cyclohexanehexol, especially a scyllo-inositol compound, and one or more NSAID, in particular R-NSAID, especially R-flurbiprofen, or a composition or conjugate comprising one or more scyllo-inositol compound and one or more NSAID, in particular R-NSAID, especially R-flurbiprofen, for preparation of a pharmaceutical composition to be employed through oral administration for treatment of a disorder characterized by abnormal protein folding and/or aggregation, and/or amyloid formation, deposition, accumulation, or persistence.

Therapeutic efficacy and toxicity of compositions, conjugates, and methods of the invention may be determined by standard pharmaceutical procedures in cell cultures or with experimental animals such as by calculating a statistical parameter such as the ED₅₀ (the dose that is therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred. One or more of the therapeutic effects, in particular beneficial effects, more particularly sustained beneficial effects disclosed herein, can be demonstrated in a subject or disease model. For example, beneficial effects may be demonstrated in a model described in the Examples herein, in particular beneficial effects may be demonstrated in a TgCRND8 mouse with symptoms of Alzheimer's disease.

### Administration

Cyclohexanehexol, NSAIDs, conjugates, and compositions of the present invention can be administered by any means that produce contact of the active agent(s) with the agent's sites of action in the body of a subject or patient to produce a therapeutic effect, in particular a beneficial effect, especially a sustained beneficial effect. The active ingredients can be administered simultaneously or sequentially and in any order at different points in time to provide the desired beneficial effects. A cyclohexanehexol, NSAID and/or composition of the invention can be formulated for sustained release, for delivery locally or systemically. It lies within the capability of a skilled physician or veterinarian to select a form and route of administration that optimizes the effects of the compositions and treatments of the present invention to provide therapeutic effects, in particular beneficial effects, more particularly sustained beneficial effects.

Cyclohexanehexols, NSAIDs, conjugates, and/or compositions may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms, all utilizing dosage forms well known to those of ordinary skill in the pharmaceutical arts. Cyclohexanehexols, NSAIDs, conjugates, and/or compositions may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen. A sustained release formulation can also be used for the therapeutic agents.

In aspects of the invention the cyclohexanehexols, NSAIDs, conjugates, and/or compositions are administered by peripheral administration, in particular by intravenous administration, intraperitoneal administration, subcutaneous administration, intramuscular administration, oral administration, topical administration, transmucosal administration, or pulmonary administration.

An amount of a cyclohexanehexol, NSAID, conjugate, composition and/or conjugate which will be effective in the treatment of a particular disease to provide effects, in particular beneficial effects, more particularly sustained beneficial effects, will depend on the nature of the disease, and can be determined by standard clinical techniques. The precise dose to be employed in the formulation will also depend on the route of administration, and the seriousness of the disease, and should be decided according to the judgment of the practitioner and each patient's circumstances. In particular, the dosage regimen of the invention will vary depending upon known factors including without limitation pharmacodynamic characteristics of the agents and their mode and route of administration, the species, age, sex, health, medical condition, and weight of the patient, the nature and extent of the symptoms, the kind of concurrent treatment, the frequency of treatment, the route of administration, the renal and hepatic function of the patient, and the desired effect.

Suitable dosage ranges for administration are selected to provide beneficial effects, particularly sustained beneficial effects. A dosage range is generally effective for triggering the desired biological responses.

The dosage ranges for the cyclohexanehexol are generally about 0.1 mg to about 2 kg per kg per day, about 0.5 mg to about 2 g per kg per day, about 1 mg to about 1 g per kg per day, about 1 mg to about 200 mg per kg per day, about 1 mg to about 100 mg per kg per day, about 10 mg to about 100 mg per kg, about 30 mg to about 70 mg per kg per day, about 1 mg to about 50 mg per kg per day, about 2 to about 50 mg/kg/day, about 2 mg to about 40 mg per kg, or about 3 mg to 30 mg per kg per day.

In aspects of the invention, the dosage ranges of a cyclohexanehexol, in particular a scyllo-inositol compound, administered once twice, three times or more daily, especially once or twice daily, are about 1 to about 100 mg/kg, 1 to about 90 mg/kg, 1 to about 80 mg/kg, 1 to about 75 mg/kg, 1 to about 70 mg/kg, 1 to about 60 mg/kg, 1 to about 50 mg/kg, 1 to about 40 mg/kg, 1 to about 35 mg/kg, 2 to about 35 mg/kg, 2.5 to about 30 mg/kg, 3 to about 30 mg/kg, 3 to about 20 mg/kg, or 3 to about 15 mg/kg. In embodiments of the invention, the required dose of cyclohexanehexol, in particular scyllo-inositol, administered twice daily is about 1 to about 50 mg/kg, 1 to about 40 mg/kg, 2.5 to about 40 mg/kg, 3 to about 40 mg/kg, 3 to about 35 mg/kg, most preferably about 3 to about 30 mg/kg. In embodiments of the invention, the required daily dose of cyclohexanehexol, in particular scyllo-inositol, is about 1 to about 80 mg/kg and within that range about 1 to about 70 mg/kg, about 1 to about 65 mg/kg, about 2 to about 70 mg/kg, about 3 to about 70 mg/kg, about 4 to about 65 mg/kg, about 5 to about 65 mg/kg, or about 6 to about 60 mg/kg.

In embodiments of the invention, the required dose of cyclohexanehexol, in particular a scyllo-inositol compound, administered twice daily is about 1 to about 50 mg/kg, 1 to about 40 mg/kg, 2.5 to about 40 mg/kg, 3 to about 40 mg/kg, 3 to about 35 mg/kg, most preferably about 3 to about 30 mg/kg.

In other embodiments of the invention, the required daily dose of cyclohexanehexol, in particular a scyllo-inositol compound, is about 1 to about 80 mg/kg and within that range 1 to about 70 mg/kg, 1 to about 65 mg/kg, 2 to about 70 mg/kg, 3 to about 70 mg/kg, 4 to about 65 mg/kg, 5 to about 65 mg/kg, or 6 to about 60 mg/kg.

A cyclohexanehexol can be provided once daily, twice daily, in a single dosage unit or multiple dosage units (i.e., tablets or capsules) having about 50 to about 10000 mg, 50 to about 2000 mg, 70 to about 7000 mg, 70 to about 6000 mg, 70 to about 5500 mg, 70 to about 5000 mg, 70 to about 4500 mg, 70 to about 4000 mg, 70 to about 3500 mg, 70 to about 3000 mg, 150 to about 2500 mg, 150 to about 2000 mg, 200 to about 2500, 200 to about 2000 mg, 200 to about 1500 mg, 700 to about 1200 mg, or 1000 mg, in particular 200 to 2000 mg, more particularly 700 to 1200 mg, most particularly 1000 mg.

In aspects of the invention, a cyclohexanehexol, in particular scyllo-inositol, is administered in an amount sufficient to result in a concentration in the CSF, brain and/or plasma of a subject of between or from about 0.05 μM to about 100 μM, 0.05 μM to about 90 μM, 0.05 μM to about 80 μM, 0.05 μM to about 70 μM, 0.05 μM to about 60 μM, 0.05 μM to about 50 μM, 0.05 μM to about 40 μM, 0.05 μM to about 30 μM, or 0.05 μM to about 20 μM. In embodiments, the concentration of the compound in CSF, brain and/or plasma is between or from about 0.1 μM to about 100 μM, 0.1 μM to about 90 μM, 0.1 μM to about 80 μM, 0.1 μM to about 70 μM, 0.1 μM to about 60 μM, 0.1 μM to about 50 μM, 0.1 μM to about 40 μM, 0.1 μM to about 30 μM, 0.1 μM to about 20 μM, or 0.1 μM to about 10 μM.

In aspects of the invention, a cyclohexanehexol, in particular scyllo-inositol, is administered in an amount sufficient to result in peak plasma concentrations, Cₘₐₓ, of from or between about 1 to about 125 μg/ml, 1 to about 100 μg/ml, 1 to about 90 μg/ml, 1 to about 80 μg/ml, 1 to about 70 μg/ml, 1 to about 60 μg/ml, 1 to about 50 μg/ml, 1 to about 40 μg/ml 1 to about 30 μg/ml, 1 to about 20μg/ml, 1 to about 10 μg/ml, 1 to about 5 μg/ml, 5 μg/ml, 5 to about 125 μg/ml, 5 to about 100 μg/ml, 5 to about 70 μg/ml, 5 to about 50 μg/ml, 10 to about 100 mg/ml, 10 to about 90 μg/ml, 10 to about 80 μg/ml, 10 to about 70 μg/ml, 10 to about 60 μg/ml, 10 to about 50 μg/ml, 10 to about 40 μg/ml, 10 to about 30μg/ml, or 10 to about 20 μg/ml. In embodiments, the Cₘₐₓ, is between or from about 1-125 μg/ml, 1-100 μg/ml, 5-70 μg/ml, 5-50 μg/ml, 10-100 μg/ml, 10-90 μg/ml, 10-80 μg/ml, 10-70 μg/ml, 10-60 μg/ml, 10-50 μg/ml or 10-40 μg/ml. In particular embodiments, the Cₘₐₓ is from or between about 5 to about 70 μg/ml, 5 to about 65 μg/ml, 5 to about 50 μg/ml, 5 to about 40 μg/ml, 5 to about 30 μg/ml, or 5 to about 20 μg/ml.

The time to achieve a desirable plasma level (t_{1/2}) of a cyclohexanehexol will depend on the individual treated, but is generally between about 1 to 100 hours, 1 to 80 hours, 1 to 70 hours, 1 to 50 hours, 1 to 42 hours, 1 to 33 hours or 3 to 50, 16 to 32, 5 to 30 hours, 10 to 30 hours, 1 to 28 hours, 1 to 25 hours, 10 to 25 hours, 1 to 24 hours, 10 to 24 hours, 13 to 24 hours, 1 to 23 hours, 1 to 20 hours,1 to 18 hours, 1 to 15 hours, 1 to 14 hours, 1 to 13 hours, 1 to 12 hours, 1 to 10 hours, 1 to 8 hours, 1 to 7 hours, 1 to 5 hours, 1 to 4 hours, 1 to 3 hours or 3 to 5 hours, in particular 1 to 5 hours or 3 to 5 hours.

A NSAID can be provided once daily, twice daily, in a single dosage unit or as multiple dosage units (i.e., tablets or capsules) having about 5 mg to about 2000 mg, 50 mg to about 1800 mg, 200 to about 1600 mg, and in particular 100 to 1000 mg, 200 to 900 mg, or 300 to 900 mg. In particular aspects, a NSAID, especially R-flurbiprofen, or a pharmaceutically acceptable salt or ester thereof, can be provided twice daily, in a single dosage unit or multiple dosage units (i.e., tablets or capsules) having 350 mg, 400 mg, 450 mg, 500 mg, 550 mg, 600 mg, 650 mg, 700 mg, 750 mg, 800 mg, or 850 mg. For example, a preferred composition of the invention comprises 400 mg R-flurbiprofen and a therapeutically effective amount of a scyllo-inositol compound, and a carrier or vehicle suitable for oral administration, e.g., in tablets or capsules. Another preferred dose is 800 mg, and a preferred composition of the invention comprises 800 mg R-flurbiprofen and a therapeutically effective amount of a scyllo-inositol compound, and a carrier or vehicle suitable for oral administration, e.g., in tablets or capsules. In a preferred embodiment, the method and composition are applied such that R-flurbiprofen is administered at 400 mg per dose, every 12 hours (i.e., twice daily), separately or along with a scyllo-inositol compound. In another preferred embodiment, the method and composition are applied such that an R-flurbiprofen is administered at 800 mg per dose every 12 hours (i.e., twice daily), separately or along with a scyllo-inositol compound.

Oral administration of a NSAID dose, twice daily for at least 4 months, preferably 8 months, and more preferably 1 year, in combination with a cyclohexanehexol, may provide an improvement or lessening of decline in cognitive function, biochemical disease marker progression, and/or plaque pathology.

Preferably, the NSIAD is a R-NSAID composition or unit dose substantially free of S-stereoisomers. In one aspect, substantially free of the S-stereoisomer means at least 90% by weight R-NSAID to 10% by weight or less of a S-NSAID of the total (S+R-NSAID) in the pharmaceutical composition. In another aspect, the ratio between R-NSAID and S-NSAID in a pharmaceutical composition is at least 95% by weight to 5% by weight. In particular, the ratio may be at least 99% by weight R-flurbiprofen to 1% by weight or less of S-flurbiprofen in the pharmaceutical composition. In an embodiment, the ratio is at least 99.9% by weight R-flurbiprofen to 0.1% by weight or less of S-flurbiprofen in the pharmaceutical composition. In an aspect, the dosage form is a tablet. In another aspect, the dosage form is a capsule.

In aspects of the invention employing R-flurbiprofen-containing compositions and combination treatments, R-flurbiprofen or a pharmaceutically acceptable salt or ester thereof is administered in an amount sufficient to result in a plasma R-flurbiprofen Cₘₐₓ of about 20 to about 150 μg per mL, particularly in an individual known to have, or that is suspected of having, Alzheimer's disease or MCI. In a specific aspect, the plasma R-flurbiprofen Cₘₐₓ is from about 30 to about 95 μg per mL. In another specific aspect, the Cₘₐₓ is from about 40 to about 80 μg per mL. In another embodiment, the Cₘₐₓ is between about 100 and about 600 μM. In a specific embodiment, the plasma Cₘₐₓ is from about 150 to about 380 μM. In another more specific embodiment, the Cₘₐₓ is from about 170 to about 240 μM. In a more specific embodiment, the individual has mild to moderate Alzheimer's disease or MCI.

In an embodiment, the R-NSAID is R-flurbiprofen or a pharmaceutically acceptable salt or ester thereof, which is administered to an individual known to have, or suspected of having Alzheimer's disease or MCI, in an amount sufficient to result in a cerebrospinal fluid R-flurbiprofen Cₘₐₓ of about 0.05 to about 7.5 μg per mL. In another embodiment, the Cₘₐₓ is from about 0.08 to about 4.5 μg per mL. In another embodiment, the R-flurbiprofen or a pharmaceutically acceptable salt or ester thereof is administered in an amount sufficient to result in a cerebrospinal fluid R-flurbiprofen Cₘₐₓ of about 2 to 30 μM, from about 3.2 μM to about 20 1M, or from about 4 μM to about 12 μM.

The time to achieve a desirable plasma NSAID Cₘₐₓ will depend upon the individual to be treated, but is generally between 0.70 to 3.75 hours. In embodiments of the invention, the tₘₐₓ (time to Cₘₐₓ) is from about 0.75 to 2.00 hours, or is from about 0.75 hour to about 1.75 hours. For example, for R-flurbiprofen, the tₘₐₓ can be about 2 hours after administration. Preferably, for R-flurbiprofen, the t_{1/2} (half-life) is from about 3.75 to about 8.5 hours. Relatively more time is expected to achieve a cerebrospinal fluid Cₘₐₓ, however, generally this Cₘₐₓ is expected to be achieved between about 1 to 6 hours after administration of a dose of a R-NSAID, especially an R-flurbiprofen.

In aspects of the compositions and methods of the invention a cyclohexanehexol is used in combination with the NSAID at therapeutically effective weight ratios of between about 1:1.5 to 1:150, preferably 1:2 to 1:50.

The combined administration of a cyclohexanehexol(s) and a NSAID(s) may require less of the generally-prescribed dose for any of the agents when used alone and or may result in less frequent administration of either or both agents. In aspects of the compositions of the invention a cyclohexanehexol and a NSAID can be present in doses that are at least about 1.1 to 1.4, 1.5, 2, 3, 4, 5, 6, 7, 8, 9, or 10 fold lower than the doses of each compound alone required to treat a disease disclosed herein.

A composition or treatment of the invention may comprise a unit dosage of at least one cyclohexanehexol and at least one NSAID to provide beneficial effects, in particular one or more of the beneficial effects (a) to (t) set out herein. A “unit dosage” or “dosage unit” refers to a unitary dose i.e., a single dose, which is capable of being administered to a patient, and which may be readily handled and packed, remaining as a physically and chemically stable unit dose comprising either the active agents as such or a mixture with one or more solid or liquid pharmaceutical excipients, carriers, or vehicles.

A subject may be treated with a cyclohexanehexol and NSAID or a composition or conjugate of the invention on substantially any desired schedule. A cyclohexanehexol, in particular a scyllo-inositol compound, and a NSAID, or a conjugate or composition of the invention, may be administered one or more times per day, in particular 1 or 2 times per day, 1, 2, 3, 4, 5 or more times per week, 1 to 20, 1 to 15, 1 to 10 or 1 to 5 times a month or continuously. However, a subject may be treated less frequently, such as every other day or once a week, or more frequently. A cyclohexanehexol, NSAID or a conjugate or composition of the invention may be administered to a subject for about or at least about 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, or 2 weeks to 24 months, periodically or continuously.

In a combination therapy to treat the diseases discussed herein, a cyclohexanehexol(s) and a NSAID(s) can be administered simultaneously or at separate intervals. When administered simultaneously the cyclohexanehexol(s) and the NSAID(s) can be incorporated into a single pharmaceutical composition, e.g., a pharmaceutical combination therapy composition. Alternatively, two or more separate compositions, i.e., one containing the cyclohexanehexol(s) and the other(s) containing the NSAID(s), can be administered simultaneously.

When separately administered, therapeutically effective amounts of compositions containing a cyclohexanehexol, and a NSAID(s) may be administered on a different schedule. One may be administered before the other as long as the time between the administrations falls within a therapeutically effective interval. A therapeutically effective interval is a period of time beginning when one of either (a) the cyclohexanehexol, or (b) NSAID(s) is (are) administered to a subject and ending at the limit of the beneficial effect in the treatment of the disease to be treated from the combination of (a) and (b). The methods of administration of the cyclohexanehexol(s), and a NSAID(s) may vary. Thus, any of the agents may be administered using appropriate routes disclosed herein, including rectally, topically, orally, sublingually, or parenterally, in particular orally.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Examples

### Example 1

The following methods can be used to study the compositions and combination treatments of the invention:
Mice. Experimental groups of TgCRND8 mice [Chishti, M. A. et al., J. Biol Chem 276, 21562-21570 (2001); Janus, C. et al., Nature 408, 979-982 (2000)] on a C3H/B6 outbred background will be initially treated with 30 mg/kg/day of a scyllo-inositol compound (AZD103/ELND005 (Elan Corporation), a scyllo-inositol) and a R-NSAID (e.g. 10 mg/kg/day of R-flurbiprofen). The studies will be repeated using doses of 5 mg/kg/day-100 mg/kg/day of a scyllo-inositol compound and/or a R-NSAID. A cohort of animals (n=10 mice per treatment arm) will be entered into the study at five months of age, and outcomes analyzed after one month of treatment. The body weight, coat characteristics and in cage behaviour will be monitored. Mannitol will be used as a negative control for potential alterations in caloric intake. Controls will also include the scyllo-inositol compound and R-NSAID alone.

Behavioural tests: Morris Water Maze testing will be performed as described in Janus, C. et al., 2000. After non-spatial pre-training, mice will undergo discrimination training for 5 days with 4-trials per day, followed by a cued visible platform to rule out general motivational, learning deficits and motor problems, and a probe trial to evaluate memory. Data will be subjected to a mixed model of repeated measures analysis of variance (ANOVA) with treatment (untreated, scyllo-inositol compound and R-NSAID treated) and genotype (TgCRND8 versus non-Tg) as ‘between-subject’ factors. Open field test for motor activity will be preformed as described previously (Vaucher, E. et al., Exp. Neurol. 175, 398-406 (2002)). Duration of walking, pausing and grooming will be analyzed as indices of spontaneous locomotor activity. Sensorimotor function will be examined with an Economex™ accelerating rotarod (Columbus Instruments, Columbus, Ohio), as described elsewhere (Mount, H. T. J, J Neurochem 88:1449-1454). The rod will be set to accelerate at a rate of 0.2 r.p.m./s, from an initial, constant speed of 5 r.p.m. Latency to fall will be recorded in four daily trials, conducted at 30 min intervals. All mice will be trained for seven days before testing. The test day performance score for each animal will be obtained by summing its latency to fall over the four trials.
Cerebral amyloid burden. Brains will be removed and one hemisphere fixed in 4% paraformaldehyde and embedded in paraffin wax in the mid sagittal plane. To generate sets of systematic uniform random sections, 5 μm serial sections will be collected across the entire hemisphere. Sets of sections at 50 μm intervals will be used for analyses (10-14 sections/set). Plaques will be identified after antigen retrieval with formic acid, and incubated with primary anti-Aβ antibody (Dako M-0872), followed by secondary antibody (Dako StreptABCcomplex/horseradish kit). End products will be visualized with DAB and counter-stained with luxol fast blue. Amyloid plaque burden will be assessed with Leco IA-3001 image analysis software interfaced with Leica microscope and Hitachi KP-M1U CCD video camera. Openlab imaging software (Improvision, Lexington, Mass.) will be then used to convert micrographs to binary images for plaque number and plaque area determinations. Vascular amyloid burden will be defined as amyloid originating from or surrounding blood vessels and will be similarly analysed.
Plasma and Cerebral Aβ Content. Hemi-brain samples will be homogenized in a buffered sucrose solution, followed by either 0.4% diethylamine/100 mM NaCl for soluble Aβ levels or cold formic acid for the isolation of total Aβ. After neutralization, the samples will be diluted and analyzed for Aβ40 and Aβ42 using commercially available kits (BIOSOURCE International). Each hemisphere will be analyzed in triplicate and the mean values ±SEM reported. Western blot analyses will be performed on all fractions using urea gels for Aβ species analyses (Wiltfang, J. et al., J Neurochem 81, 481-496 (2002)). Aβ will be detected using 6E10 (BIOSOURCE International) and Enhanced Chemiluminenscence (Amersham).
Gliosis Quantitation. Five randomly selected, evenly spaced, sagittal sections will be collected from paraformaldehyde-fixed and frozen hemispheres of treated and control mice.

Sections will be immunolabelled for astrocytes with anti-rat GFAP IgG₂ₐ (Dako; diluted 1:50) and for microglia with anti-rat CD68 IgG_{2b} (Dako; 1:50). Digital images will be captured using a Coolsnap digital camera (Photometrics, Tuscon, Ariz.) mounted to a Zeiss, Axioscope 2 Plus microscope. Images will be analysed using Openlab 3.08 imaging software (Improvision, Lexington Mass.).
Survival Census: The probability of survival will be assessed by the Kaplan-Meier technique (Haccou, P., & Mellis, E., Statistical Analysis of Behavioural Data, pg 120-186, Oxford University Press, Oxford (1995)), computing the probability of survival at every occurrence of death, thus making it suitable for small sample sizes. The Tarone-Ware test will be used to assess effects of treatments.
Analysis of APP in brain. Mouse hemi-brain samples will be homogenized and spun at 109,000×g, in 20 mM Tris pH7.4, 0.25M sucrose, 1 mM EDTA and 1 mM EGTA, and a protease inhibitor cocktail, will be mixed with 0.4% DEA (diethylamine)/100 mM NaCl. The supernatants will be analysed for APPs levels by Western blotting using mAb 22C11, while the pellets will be analysed for APP holoprotein with mAb C1/6.1 as previously described (Janus, 2000, supra; Chishti, M, 2001, supra).
Soluble Aβ oligomer Analyses. The levels of soluble Aβ oligomers will be measured by a dot blot assay with anti-oligomer specific antibodies (Kayed, R. et al., Science 300, 486-489 (2003)). Briefly, oligomers will be solubilised from one hemi-brain in PBS in the presence of protease inhibitor cocktail (Sigma). After centrifugation at 78,500×g for 1 hr at 4° C., the supernatants will be analysed. Protein content will be determined by the BCA protein assay (Pierce). Two μg of total protein will be spotted onto nitrocellulose, blocked with 10% non-fat milk in TBS before incubation with a biotinylated oligomeric specific antibody. Blots will be incubated with streptavidin-HRP and ECL chemiluminescence kit. Soluble and fibrillar Aβ42 will be used as negative controls and synthetic oligomeric Aβ42 will be used as a positive control (Shetty, H. U. & Hollway, H. W., Biol. Mass Spec. 23, 440-444 (1994)). Control samples will be re-identified after oligomeric antibody is stripped and re-probed with the anti-Aβ antibody 6E10.
Long Term Potentiation. Field potentials will be recorded in CA1 of mouse hippocampus by standard procedures (Sarvey J M, et al., J Neurosci Methods 28,109-124 (1989); Stanton P K & Sarvey J M., Brain Res Bull. 18, 115-119 (1987)). Swiss Webster mice between the ages of P16 and P26 will be anesthetized with isoflurane. The brain will be rapidly removed and placed in ice cold oxygenated sucrose-CSF containing (in mM): 248 sucrose, 2 KCl, 2 MgSO₄, 1.24 NaH₂PO₄, 1 CaCl₂, 1 MgCl₂, 26 NaHCO₃, 10 D-glucose, pH 7.4, ˜315 mOsmol. (Moyer J R Jr, & Brown T H., J Neurosci Methods. 86, 35-54 (1998)). The hippocampus from each hemisphere will be isolated and 350 μm coronal sections will be made. The slices will be transferred to a holding chamber containing NaCl—CSF (in mM: 124 NaCl, 2 KCl, 2 MgSO₄, 1.25 NaH₂PO₄, 2 CaCl₂, 26 NaHCO₃, 10 D-glucose, pH 7.4, ˜310 mOsmol) and allowed to recover for more than 1 hour. Once placed in the chamber, slices will be continuously perfused by a closed loop containing 15 ml of ACSF to conserve the oligomeric Aβ. After 20 minutes of stable baseline, 1 ml of 15× concentrated 7PA2 conditioned medium±1.25 μM of each of the test compounds will be added to the perfusion loop. A bipolar stimulating electrode (World Precision Inst.) will be placed in the Schaffer collaterals to deliver baseline stimuli and tetani. A borosilicate glass recording electrode (2-4 MΩ) containing ACSF will be positioned approximately 75-200 μm from the stimulating electrode. The intensity of the stimulus (typically between 10-20 μAmps) will be set to obtain 25-40% of the maximal field potential response. Test stimuli will be delivered at 0.05 Hz. To induce LTP, 4 tetani (100 Hz for 1 second) will be delivered 5 minutes apart. Field potential responses will be amplified 10× using an Axopatch 200B. The data will be sampled at 10 kHz and filtered at 2 kHz. Traces will be analyzed using pClamp 9.2. The slope of the field potential will be estimated using approximately 10-60% of the total response.

### Synaptophysin Quantification.

Synaptophysin immunohistochemical staining will be performed on 3 evenly spaced saggital sections of paraformaldehyde-fixed treated and control mice. Sections will be immunolabelled for synaptophysin with anti-synaptophysin IgG (1:40; Roche, Laval, PQ). Digital images will be captured and analyzed as described above. Within each section, three randomly chosen 100 μm² areas of the CA1 region of the hippocampus will be counted for synaptophysin reactive cell bodies and boutons. The results will be expressed as the mean of the number of reactive bodies and boutons per 100 μm² (Phinney, A. et al., Neuroscience 90, 1207-1216 (1999); Hu, L. et al., Neuroscience 121, 421-432 (2003)).

### Discussion

To assess their effectiveness in vivo, a scyllo-inositol compound in combination with a R-NSAID inihibitor disclosed herein will be administered to a murine model of Alzheimer's disease (TgCRND8) (Chishti, M. A. et al., J. Biol Chem 276, 21562-21570 (2001); Janus, C. et al., Nature 408, 979-982 (2000)). The TgCRND8 mice and non-transgenic littermates will be assigned to sex- and age-matched cohorts that are then used to test the effectiveness of the combination therapy. The mice will be randomly assigned to receive active compounds, mock therapy (mannitol), or no therapy. The endpoints will be cognitive function, brain Aβ levels, and neuropathology.

To assess whether a combination of a scyllo-inositol compound and a R-NSAID could abrogate a well-established AD-like phenotype, the start of treatment of the TgCRND8 mice will be delayed until five months of age. At this age, TgCRND8 mice have significant behavioural deficits, accompanied by profuse Aβ peptide and plaque burdens (Chishti, M. A. et al., (2001)). Cohorts of TgCRND8 and non-Tg littermates (10 mice per cohort) will be either treated for 28 days with a scyllo-inositol compound and a R-NSAID, each compound alone, or will be left untreated. The dosage and oral administration of compounds, and the neurochemical and neuropathological assays used for these experiments will be the same as employed in the initial prophylactic experiments.

Spatial learning in the transgenic mice will be compared between six month old TgCRND8 mice that have been treated with a compound disclosed herein or that were untreated for 28 days. The performance of six month old TgCRND8 mice that had been treated with a scyllo-inositol compound and a R-NSAID for 28 days is anticipated to result in better behavioural performance. A 28 day course of treatment at 5 months of age is also expected to: 1) reduce brain levels of Aβ40 and Aβ42; and, 2) significantly reduce plaque number, plaque size, and percent area of the brain covered in plaques. The results are expected to demonstrate an additive or synergistic effect compared to each compound alone.

To directly address the possibility that the compounds inhibit Aβ oligomerization in the brain, a dot blot immunoassay (Kayed, R. et al., Science 300, 486-489 (2003)) will be used to measure levels of Aβ oligomers in the brains of treated and untreated TgCRND8 mice. The assay employs an antibody that selectively identifies oligomeric Aβ species (Kayed, R. et al., 2003, supra). The levels of soluble Aβ oligomers are expected to be significantly reduced in the brain of mice treated with a combination disclosed herein. The results are expected to demonstrate an additive or synergistic effect, especially a synergistic effect, compared to each compound alone.

To address the possibility that the compounds inhibit Aβ oligomer-induced neurotoxicity, their effects will be determined on both long term potentiation (LTP) in mouse hippocampal slices and on synaptic density as measured by the level of synaptophysin immunoreactivity in the brains of TgCRND8 mice. Hippocampal LTP is a measure of synaptic plasticity, and has been shown to be disrupted by natural cell-derived oligomeric Aβ species (Walsh, D. M. et al., Nature 416, 535-539 (2002)). As previously reported in rat (Walsh, D. M. et al., Nature 416, 535-539 (2002); Walsh, D. M., et al, Biochem Soc. Trans. 30, 552-557 (2002)), soluble Aβ oligomers secreted into the conditioned media of CHO cells stably transfected with human APPV717F (7PA2 cells) inhibited LTP in wild-type mouse hippocampal slices. However, it is anticipated that when the 7PA2-conditioned medium is pretreated in vitro with a scyllo-inositol compound and a R-NSAID there will be enhanced recovery of LTP compared with 7PA2-conditioned media alone or the media with each compound alone. In order to correlate the protection of LTP in slice cultures with in vivo effects on synaptic function, the level of synaptophysin immunoreactivity will be measured in the CA1 region of the hippocampus in treated and untreated TgCRND8 mice. Synaptophysin immunoreactivity is a measure of synaptic density, which is correlated to synaptic function. The levels of synaptophysin are expected to be increased. Thus, compounds are expected to increase the number of synaptophysin reactive boutons and cell bodies in the CA1 region of the hippocampus. The results are expected to demonstrate an additive or synergistic effect, especially synergistic effect, compared to each compound alone.

### Example 2

Clinical Study for Alzheimer's Disease using a Combination of Scyllo-Inositol and R-NSAID.

A R-NSAID in combination with scyllo-inositol may be studied for its actions in healthy subjects and subjects with mild to moderate Alzheimer's disease (AD). The evaluation of a R-NSAID and scyllo-inositol for treating Alzheimer's may be carried out in a three-group parallel design study; each group having 50 subjects for a total of 150 subjects. Subjects will be treated with scyllo-inositol and a R-NSAID (e.g. R-flurbiprofen) and or a matching placebo twice a day for forty-eight weeks.

AD subjects may be selected based on the following criteria. Subjects (1) are men and women, ages 60-85, who are diagnosed with probable AD using the National Institute of Neurologic Communicative Disorders and Stroke-Alzheimer's Disease and Related Disorders Association (NINCDS-ADRDA) test (McKhann et al. (1984) Neurology 34:939-944), (2) have mild to moderate dementia as determined by the Mini-Mental State Examination (MMSE, Mohs et al. (1996) Int Psychogeriatr 8:195-203) with MMSE scores in the range of 15-25 indicating mild to moderate dementia; and (3) have caregivers that can ensure compliance with medication regimens and with study visits and procedures.

Control subjects will consist of men and women ages 60-80 that lack significant cognitive or functional complaints, or depression as determined by the Geriatric Depression Scale (GDS), and have MMSE scores in the range of 27-30.

Subjects that will be excluded from the study include: (1) those who have medically active major inflammatory co-morbid condition(s) such as rheumatoid arthritis, or those who have peptic ulcer, gastro-intestinal bleeding, or intolerance of NSAIDs in the past; (2) those who have contra-indications to lumbar puncture, such as severe lumbar spine degeneration, sepsis in the region of the lumbar spine, or a bleeding disorder; (3) those who use medications such as prednisone, or immunosuppressive medications such as cyclophosphamide that could interfere with the study; (4) those undergoing acetylcholinsterase inhibitor (AChE-I) treatments for AD that have not been on stable doses for at least four weeks and; (5) those AD subjects taking antioxidants such as vitamin E, vitamin C, or Gingko biloba if they have not been on stable doses for at least four weeks.

The primary efficacy endpoint may be the rate of decline in the ADAS-cog score. The decline may be based on either a slope calculated for each patient or on a Generalized

Estimating Equations (GEE) model. Secondary efficacy endpoints may include scores on the CIBIC+, NPI, ADCS-ADL, and CDR sum of boxes.

The study protocol may involve three in-clinic visits: an initial screening visit, a baseline visit, and a follow-up visit. During the screening visit, information needed to assess eligibility may be obtained and an MMSE may be administered. The treatment regimen may consist of treatments with NSAIDs in the form of capsules taken two or three times a day with meals. Both a high and a low study dose of NSAID may be used. For example, in the case of ibuprofen doses of 800 mg (e.g. two 400 mg ibuprofen tablets) and 400 mg (e.g. one 400 mg ibuprofen capsule and one placebo capsule) may be used in the study. If R-flurbiprofen is used in the study, both high (e.g. 800 mg) and low (e.g., 400 mg) doses may be used. If sulindac is used in the study, a dose of 200 mg twice a day for a total of 400 mg per day may be used. If meclofenamic acid is used in the study doses of 100 mg and 400 mg per day may be tested. Treatment with scyllo-inositol may be a once or twice daily dosing regimen, in particular 500 to 4000,1000 to 4000 mg or 500 to 2000 mg, in particular 1000 mg twice per day.

During the baseline visit, which takes place within two weeks of the screening visit, physical examinations and lumbar punctures are performed. Blood samples are drawn for laboratory tests such as APO-E genotyping and for plasma preparation. At this time, subjects or caregivers, in the case of AD subjects, are given a supply of study NSAID and scyllo-inositol, along with instructions about timing of doses and potential adverse effects.

During the follow-up visit (e.g. 12-14 days following the start of treatment), vital signs and adverse side effects of the study drugs may be assessed. Lumbar punctures may be performed and blood samples may be drawn for laboratory tests and for plasma preparations. Biological markers that may be analyzed from plasma and CSF samples using methods within the skill of an ordinary artisan include without limitation plasma protein, glucose, Aβ40, Aβ42, Aβ38, Tau, M-CSF, MCP-1, and P-tau181.

Patients having mild-to-moderate Alzheimer's disease undergoing the treatment regimen of this example with a R-NSAID in doses of about 10 mg to 1600 mg per day, and a scyllo-inositol are expected to experience a lessening in decline of cognitive function (as measured by ADAS-cog or CDR sum of boxes), plaque pathology, and/or biochemical disease marker progression.

### Example 3

Prevention of Alzheimer's Disease with a R-NSAID and Scyllo-Inositol Combination

Prior to the onset of symptoms of Alzheimer's disease or at the very early stages of the disease, subjects desiring prophylaxis against Alzheimer's disease may be treated with a combination of a R-NSAID and a scyllo-inositol. Subjects needing prophylaxis may be assessed by monitoring disease markers, detecting genes conferring a predisposition to Alzheimer's disease, and detecting other risks factors such as age, diet, and other disease conditions associated with Alzheimer's disease. A subject desiring prophylaxis against Alzheimer's disease or prophylaxis of a worsening of the symptoms of Alzheimer's disease may be treated with a R-NSAID and a scyllo-inositol in an amount sufficient to delay the onset or progression of symptoms of Alzheimer's disease. For example, a patient may be treated with 800 mg of a R-NSAID (e.g., R-flurbiprofen) twice daily and once or twice daily with a scyllo-inositol (eg. about 1 mg to 100 mg per kg, about 1 mg to 50 mg per kg body weight, or about 3 mg to 30 mg per kg body weight, or with a tablet or dosage unit having from about 100 to 5000 mg, 200 to 2000 mg, 1000 to 4000 mg or 500 to 2000 mg, in particular 1000 mg).

Alternatively, a R-NSAID and scyllo-inositol may be formulated in a single tablet for administration once or twice daily to a subject. Another prophylactic regimen involves administering to the patient 400 mg of a R-NSAID (e.g., R-flurbiprofen) twice daily and twice daily with a scyllo-inositol (i.e. a total daily or twice daily dosage of from about 1 mg to 100 mg per kg, about 1 mg to 50mg per kg, or about 3 mg to 30 mg per kg). The amounts of active ingredients may be modified to lessen side-effects and/or produce the most therapeutic benefit. For example, lower doses of a R-NSAID (e.g., 200 mg of R- flurbiprofen twice daily) can be administered to reduce sides effects associated with the use of higher levels of the active ingredient. The preventive treatment can also involve treating on alternating days or weeks with a R-NSAID.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the models, methodologies etc. which are reported therein which might be used in connection with the invention. Nothing herein is to be construed as an admission that the invention is not entitled to antedate such disclosure by virtue of prior invention.

TABLE 1

| NSAID | Commercial Source |
|---|---|
| Racemates of ketoprofen, flurbiprofen, | Sigma-Aldrich |
| carprofen, indoprofen | |
| Sodium salt of R-naproxen | Sigma-Aldrich |
| R-ketoprofen, R-flurbiprofen, | Sepracor, Inc. |
| R-ketorolac | |
| R-flurbiprofen | DSM Pharmaceutical Products; |
| | Encore Pharmaceutical Inc. |
| R-etodolac (Ultradol ™, Lodine ™) | Wyeth-Ayerst |
| R-tiaprofenic | Roussel |
| R-suprofen | McNiel Pharmaceuticals |
| R-carprofen | Roche |
| R-pirprofen | Ciba |
| R-indoprofen | Carlo Elba, Italy, UK |
| R-benoxaprofen | Eli Lilly Co. |
| Flurizan ™ | Myriad Genetics Inc. |
| R-ketoprofen, Orudis, Oruvail, Actron, | Elan Pharmaceuticals; ANDRX |
| Orudis KT | Pharmaceuticals, Mylan, Wyeth |
| | Pharmaceuticals, Clonmel |
| | Healthcare, Sandoz, Teva |

1. (canceled)

2. A pharmaceutical composition as claimed in claim 3, wherein the cyclohexanehexol and NSAID are present in therapeutically effective amounts at or adjacent to a site of administration of the pharmaceutical composition and at a time of administration sufficient to provide a synergistic therapeutic effect on preventing or reducing aggregation of Aβ, maintaining synaptic function, and/or reducing Aβ load.

3. A pharmaceutical composition comprising a cyclohexanehexol of the formula Va or Vb: 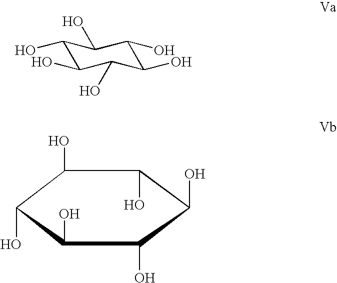
wherein optionally one, two, three, four, five or six hydroxyl groups are replaced by monovalent substituents, with retention of configuration and at least one NSAID that provides synergistic effects relative to each compound alone.

4. (canceled)

5. A pharmaceutical composition as claimed in claim 3 wherein one or two hydroxyl groups in the cyclohexanehexol are replaced with hydrogen; alkyl; substituted alkyl; acyl; alkenyl; substituted alkenyl; alkynyl; substituted alkynyl; cycloalkyl; substituted cycloalkyl; alkoxy; substituted alkoxy; aryl; aralkyl; substituted aryl; halogen; —NHR⁴¹ wherein R⁴¹ is hydrogen, acyl, alkyl; —NR⁴²R⁴³ wherein R⁴² and R⁴³ are the same or different and represent acyl or alkyl; —PO₃H₂; —SR⁴⁴ wherein R⁴⁴ is hydrogen, substituted or unsubstituted alkyl, or —O₃H; or OSO₃H.

6. A pharmaceutical composition as claimed in claim 3 wherein the cyclohexanehexol and the NSAID are present in doses that are at least about 1.1 to 1.4, 1.5, 2, 3, 4, 5, 6, 7, 8, 9, or 10 fold lower than the doses of each compound alone required to treat a disorder in protein folding and/or aggregation, and/or amyloid formation, deposition, accumulation, or persistence.

7. A pharmaceutical composition according to claim 3 comprising about 50 to about 10000 mg, 50 to about 2000 mg, 70 to about 7000 mg, 70 to about 6000 mg, 70 to about 5500 mg, 70 to about 5000 mg, 70 to about 4500 mg, 70 to about 4000 mg, 70 to about 3500 mg, 70 to about 3000 mg, 150 to about 2500 mg, 150 to about 2000 mg, 200 to about 2500, 200 to about 2000 mg, or 200 to about 1500 mg, 700 to about 1200 mg or 1000 mg of the cylcohexanehexol.

8. A pharmaceutical composition according to claim 3 comprising about 5 mg to about 2000 mg, 50 mg to about 1800 mg, 200 to about 1600 mg, 100 to about 1000 mg, 200 to about 900 mg, or 300 to about 900 mg of the NSAID.

9. A pharmaceutical composition according to claim 3 wherein the NSAID is an R-NSAID.

10. A pharmaceutical composition according to claim 9 wherein the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-etodolac and R-benoxaprofen.

11. A pharmaceutical composition according to claim 9 wherein the R-NSAID is a compound listed in Table 1.

12. (canceled)

13. A unit dosage form comprising a cyclohexanhexol and at least one NSAID wherein the dosage of cylcohexanehexol is about 50 to about 10000 mg, 50 to about 2000 mg, 70 to about 7000 mg, 70 to about 6000 mg, 70 to about 5500 mg, 70 to about 5000 mg, 70 to about 4500 mg, 70 to about 4000 mg, 70 to about 3500 mg, 70 to about 3000 mg, 150 to about 2500 mg, 150 to about 2000 mg, 200 to about 2500, 200 to about 2000 mg, 200 to about 1500 mg, 700 to about 1200 mg or 1000 mg, and the dosage of NSAID is about 5 mg to about 2000 mg, 50 mg to about 1800 mg, 200 to about 1600 mg, 100 to about 1000 mg, 200 to about 900 mg, or 300 to about 900 mg of the NSAID.

14. (canceled)

15. A method of treatment of a neurodegenerative disease comprising administering to a subject in need thereof a therapeutically effective amount of at least one cyclohexanehexol of the formula Va, Vb as defined in claim 3 in combination with administration of at least one NSAID.

16. A method as claimed in claim 15 wherein the neurodegenerative disease is Alzheimer's disease, dementia, MCI, Huntington's disease, multiple sclerosis, Parkinson's disease, amyotrophic lateral sclerosis, epilepsy, or Pick's disease.

17. (canceled)

18. A method according to claim 15 wherein therapeutically effective amounts of the cyclohexanehexol and the NSAID are combined prior to administration to the subject.

19. A method according to claim 15 wherein therapeutically effective amounts of the cyclohexanehexol and the NSAID are administered to the subject sequentially.

20. A method according to claim 15 wherein the therapeutically effective amounts of the cyclohexanehexol and the NSAID are synergistically effective amounts.

21. A method according to claim 15 wherein the NSAID is a R-NSAID selected from the group consisting of R-flurbiprofen, R-ibuprofen, R-ketoprofen,
R-ketorolac, R-naproxen, R-tiaprofenic acid, R-suprofen, R-carprofen, R-pirprofen, R-indoprofen, R-etodolac and R-benoxaprofen.

22. A method according to claim 15 wherein the neurodegenerative disease is Alzheimer's disease.

23. A method according to claim 15 wherein the NSAID is a flurbiprofen and the cyclohexanehexol is a scyllo-inositol compound.

24. (canceled)

25. (canceled)
